(19) United States
(12) Patent Application Publication
(10) Pub. No.: US 2025/0059603 A1
(43) Pub. Date: Feb. 20, 2025

(54) EXPANDED PANELS OF NUCLEIC ACID BIOMARKERS FOR PLACENTAL DYSFUNCTION

(71) Applicants: Sera Prognastics, Inc., Salt Lake City, UT (US); The Regents of the University of California, Oakland, CA (US)

(72) Inventors: John Jay Boniface, Salt Lake City, UT (US); Julja Burchard, Holladay, UT (US); Ryan Michael Treacy, Salt Lake City, UT (US); Louise C. Laurent, La Jolla, CA (US); Srimeenakshi Srinivasan, San Diego, CA (US)

(21) Appl. No.: 18/720,587

(22) PCT Filed: Dec. 15, 2022
(86) PCT No.: PCT/US2022/053089

§ 371 Date: Jun. 14, 2024

Related U.S. Application Data
(60) Provisional application No. 63/361,398, filed on Dec. 15, 2021.

(51) Int. Cl. C12Q 1/6883; C12Q 1/6874; G16B 20/00

(52) U.S. Cl. CPC C12Q 1/6883; C12Q 1/6874; G16B 20/00; C12Q 2600/118; C12Q 2600/178

(57) ABSTRACT

The present invention provides compositions and methods for determining a pregnant female's risk of developing placental dysfunction later in the pregnancy. The present invention also provides compositions and methods for determining a pregnant female's risk of developing placental early in the pregnancy or throughout the pregnancy.

### CROSS-REFERENCE TO RELATED APPLICATION

This application claims priority under 35 U.S.C. § 119 (e) and the Paris Convention to U.S. Provisional Application No. 63/361,398, filed Dec. 15, 2021, the contents of which are incorporated herein by reference in their entireties.

### BACKGROUND

Placental dysfunction, most commonly manifested as preeclampsia or intrauterine growth restriction, is an important cause of maternal and fetal morbidity and mortality in both the developing and developed world. placental insufficiency. In particular, placental dysfunction linked to preterm birth (PTB), preeclampsia, intrauterine growth restriction, preterm labor, preterm premature rupture of membranes, late spontaneous abortion and abruption placentae. It is thought that placental dysfunction arises from abnormal trophoblast differentiation and/or invasion, events that occur in the first trimester of pregnancy, but become clinically apparent only in the late second and third trimesters. Optimal surveillance and management of placental dysfunction, as well as the development of effective therapies, have been hampered by the lack of methods for early and accurate identification of pregnancies at risk for this disorder. MicroRNAs (miRNAs) are non-coding, 21-25 nucleotide, regulatory RNAs that affect the stability and/or translational efficiency of messenger-RNA (mRNAs). There is a need to develop a maternal blood-based assay for quantification of extracellular microRNA (miRNA) biomarkers present in the maternal serum during the second trimester of pregnancy in order to determine a pregnant female's risk of developing placental dysfunction later in the pregnancy. The present invention addresses this need and provides related advantages.

### SUMMARY

The present invention provides compositions and methods for determining a pregnant female's risk of developing placental dysfunction later in the pregnancy.

In one aspect, this disclosure provides a panel of isolated nucleic acid biomarkers comprising a combination of nucleic acid biomarker pairs as listed in Table 6.

In some embodiments, the combination of nucleic acid biomarker pairs described herein comprises hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, the combination of nucleic acid biomarker pairs described herein comprises hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, the combination of nucleic acid biomarker pairs described herein comprises hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, the combination of nucleic acid biomarker pairs described herein comprises hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, the combination of nucleic acid biomarker pairs described herein comprises hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, the combination of nucleic acid biomarker pairs described herein comprises hsa-miR-7-5p/hsa-miR-485-5p; hsa-miR-1306-3p/hsa-miR-664a-5p; and hsa-miR-501-3p/hsa-miR-484.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, the combination of nucleic acid biomarker pairs described herein comprises hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, the combination of nucleic acid biomarker pairs described herein comprises hsa-miR-7-5p/hsa-miR-485-5p; hsa-miR-1306-3p/hsa-miR-664a-5p; and hsa-miR-628-3p/hsa-miR-19b-3p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, the combination of nucleic acid biomarker pairs described herein comprises hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, the combination of nucleic acid biomarker pairs described herein comprises hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, the combination of nucleic acid biomarker pairs described herein comprises hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p.

In some embodiments, the combination of nucleic acid biomarker pairs described herein comprises hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, the combination of nucleic acid biomarker pairs described herein comprises hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, the combination of nucleic acid biomarker pairs described herein comprises hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, the combination of nucleic acid biomarker pairs described herein comprises hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, the combination of nucleic acid biomarker pairs described herein comprises hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, the combination of nucleic acid biomarker pairs described herein comprises hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, the combination of nucleic acid biomarker pairs described herein comprises hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, the combination of nucleic acid biomarker pairs described herein comprises hsa-miR-7-5p/hsa-miR-485-5p; hsa-miR-30c-5p/hsa-miR-125a-5p; and hsa-miR-1-3p/hsa-miR-345-5p.

In some embodiments, the combination of nucleic acid biomarker pairs described herein comprises hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, the combination of nucleic acid biomarker pairs described herein comprises hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p.

In some embodiments, the combination of nucleic acid biomarker pairs described herein comprises hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, the combination of nucleic acid biomarker pairs described herein comprises hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, the combination of nucleic acid biomarker pairs described herein comprises hsa-miR-7-5p/hsa-miR-485-5p; hsa-miR-1260b/hsa-mir-320c-2; and hsa-miR-433-3p/hsa-miR-345-5p.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, the combination of nucleic acid biomarker pairs described herein comprises hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, the combination of nucleic acid biomarker pairs described herein comprises hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, the combination of nucleic acid biomarker pairs described herein comprises hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, the combination of nucleic acid biomarker pairs described herein comprises hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, the combination of nucleic acid biomarker pairs described herein comprises hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, the combination of nucleic acid biomarker pairs described herein comprises hsa-miR-7-5p/hsa-miR-485-5p; hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, the combination of nucleic acid biomarker pairs described herein comprises hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, the combination of nucleic acid biomarker pairs described herein comprises hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, the combination of nucleic acid biomarker pairs described herein comprises hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, the combination of nucleic acid biomarker pairs described herein comprises hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, the combination of nucleic acid biomarker pairs described herein comprises hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, the combination of nucleic acid biomarker pairs described herein comprises hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, the combination of nucleic acid biomarker pairs described herein comprises hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, the combination of nucleic acid biomarker pairs described herein comprises hsa-miR-7-5p/hsa-miR-485-5p; hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, the combination of nucleic acid biomarker pairs described herein comprises hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the combination of nucleic acid biomarker pairs described herein comprises hsa-miR-7-5p/hsa-miR-485-5p; hsa-miR-221-5p/hsa-miR-760; hsa-miR-99b-3p/hsa-miR-340-5p; and hsa-miR-1306-3p/hsa-miR-150-3p.

In some embodiments, the combination of nucleic acid biomarker pairs described herein comprises hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, the combination of nucleic acid biomarker pairs described herein comprises hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, the combination of nucleic acid biomarker pairs described herein comprises hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the combination of nucleic acid biomarker pairs described herein comprises hsa-miR-330-5p/hsa-miR-223-3p; hsa-miR-7-5p/hsa-miR-485-5p; and hsa-miR-511-5p/hsa-miR-148a-5p.

In some embodiments, the combination of nucleic acid biomarker pairs described herein comprises hsa-miR-7-5p/hsa-miR-485-5p; hsa-miR-1306-3p/hsa-miR-664a-5p; hsa-miR-30a-5p/hsa-miR-941; and hsa-miR-30c-5p/hsa-miR-27b-3p.

In some embodiments, the combination of nucleic acid biomarker pairs described herein consists of one or more of the combinations of panels of nucleic acid biomarker pairs found in Table 6.

In some embodiments, the combination of nucleic acid biomarker pairs described herein consists of one of the combinations of panels of nucleic acid biomarker pairs found in Table 6.

In some embodiments, the combination of nucleic acid biomarker pairs described herein consists of two of the combinations of panels of nucleic acid biomarker pairs found in Table 6.

In some embodiments, the combination of nucleic acid biomarker pairs described herein consists of three of the combinations of panels of nucleic acid biomarker pairs found in Table 6.

In another aspect, this disclosure provides a composition of labeled and/or amplified nucleic acid molecules, wherein said labeled and/or amplified nucleic acid molecules correspond to any one or more of the panels of isolated nucleic acid biomarkers of Table 6.

In a different aspect, this disclosure provides a method for determining a pregnant female's risk of developing placental dysfunction during pregnancy comprising measuring amounts of the combination of nucleic acid biomarker pairs from the panel of isolated nucleic acid biomarkers of any one or more of the panels from Table 6 in a biological sample obtained from the pregnant female, calculating a risk score based upon the measured amounts to determine the pregnant female's risk of developing placental dysfunction, and providing a risk score corresponding to the pregnant female's risk of developing placental dysfunction.

Accordingly, this disclosure can provide a method for determining a pregnant female's risk of developing placental dysfunction during pregnancy comprising producing labeled and/or amplified nucleic acid molecules that correspond to the composition of labeled and/or amplified nucleic acid molecules of the one or more panels of Table 6 in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

In some embodiments, this disclosure provides a method of determining a pregnant female's risk of developing placental dysfunction, wherein the biological sample is obtained between 17 and 29 weeks of gestational age at blood draw.

In some embodiments, this disclosure provides a method of determining a pregnant female's risk of developing placental dysfunction, wherein the biological sample is obtained between 19 and 27 weeks of gestational age at blood draw.

In some embodiments, this disclosure provides a method of determining a pregnant female's risk of developing placental dysfunction, wherein the biological sample is obtained between 17 and 36 weeks of gestational age at blood draw.

In some embodiments, this disclosure provides a method of determining a pregnant female's risk of developing placental dysfunction, wherein the biological sample is obtained more than 17, more than 18, more than 19, more than 20, more than 21, more than 22, more than 23, more than 24, more than 25, more than 26, more than 27, more than 28, more than 29, more than 30, more than 31, more than 32, more than 33, more than 34, or more than 35 weeks of gestational age at blood draw.

In some embodiments, this disclosure provides a method of determining a pregnant female's risk of developing placental dysfunction, wherein the biological sample is obtained between 119-153 days, 154-196 days, and/or 119-202 days.

In some embodiments, this disclosure provides a method of determining a pregnant female's risk of developing placental dysfunction, wherein the risk score is calculated based on a ratio of data values. In some embodiments, data transformation is applied before or after the ratio is determined.

In some embodiments, this disclosure provides a method of determining a pregnant female's risk of developing placental dysfunction, wherein the method determines the pregnant female's risk of developing placental dysfunction early in the pregnancy. In some embodiments, early pregnancy is between 119-153 days of gestation.

In other embodiments, this disclosure provides a method of determining a pregnant female's risk of developing placental dysfunction, wherein the method determines the pregnant female's risk of developing placental dysfunction later in the pregnancy. In some embodiments, late pregnancy is between 154-196 days of gestation.

In another aspect, this disclosure provides a method of treating or preventing placental dysfunction in a pregnant female, the method comprising: (a) detecting the panel of isolated nucleic acid biomarkers of any one or more of the panels of nucleic acid biomarkers listed in Table 6 in a biological sample obtained from a pregnant female; (b) calculating a risk score based upon measured levels of the isolated nucleic acid biomarkers; and (c) administering one or more therapies to the pregnant female based on the risk score, to treat or prevent placental dysfunction. In some embodiments, the method further comprises a step of determining gestational age at blood draw (GABD). In some embodiments, determining GABD is performed before the detecting step.

In a different aspect, this disclosure provides a method of treating or preventing placental dysfunction in a pregnant female, the method comprising: (a) detecting the composition of labeled and/or amplified nucleic acid molecules of any one or more of the panels of nucleic acid biomarkers listed in Table 6 from a biological sample obtained from a pregnant female; (b) calculating a risk score based upon measured levels of the labeled and/or amplified nucleic acid molecules; and (c) administering one or more therapies to the pregnant female based on the risk score, to treat or prevent placental dysfunction. In some embodiments, the method further comprises a step of determining gestational age at blood draw (GABD). In some embodiments, determining GABD is performed before the detecting step.

In some embodiments, this disclosure provides a method of treating or preventing placental dysfunction in a pregnant female, wherein the nucleic acid biomarkers are normalized. For example, in some embodiments, normalization comprises a reversal of the isolated nucleic acid biomarkers.

In some embodiments, this disclosure provides a method of treating or preventing placental dysfunction in a pregnant female, wherein the reversal comprises an upregulated biomarker and a downregulated biomarker. In some embodiments, the risk score is calculated based on the reversal of data values.

In some embodiments, this disclosure provides a method of treating or preventing placental dysfunction in a pregnant female, wherein GABD is between 119-153 days, 154-196 days, and/or 119-202 days.

In some embodiments, this disclosure provides a method of treating or preventing placental dysfunction in a pregnant female, wherein the method determines a pregnant female's risk of developing placental dysfunction across the full window of gestation. In some embodiments, the full gestation window of gestation is between 119-202 days of gestation.

In some embodiments, this disclosure provides a method of treating or preventing placental dysfunction in a pregnant female, wherein the method determines a pregnant female's risk of developing placental dysfunction early in the pregnancy. In some embodiments, early pregnancy is between 119-153 days of gestation.

In some embodiments, this disclosure provides a method of treating or preventing placental dysfunction in a pregnant female, wherein the method determines a pregnant female's risk of developing placental dysfunction later in the pregnancy. In some embodiments, late pregnancy is between 154-196 days of gestation.

In some embodiments, this disclosure provides a method of treating or preventing placental dysfunction in a pregnant female, wherein the placental dysfunction is preeclampsia.

In some embodiments, this disclosure provides a method of treating or preventing placental dysfunction in a pregnant female, wherein calculating the risk score is performed using a binary decision tree analysis.

In some embodiments, this disclosure provides a method of treating or preventing placental dysfunction in a pregnant female, wherein the binary decision tree analysis comprises comparing reversal values of two or more biomarker pairs to two or more predetermined cutoff values and assigning the risk score based on the results of said comparison.

In some embodiments, this disclosure provides a method of treating or preventing placental dysfunction in a pregnant female, wherein calculating the risk score comprises comparing a reversal value of a first biomarker pair from the panel to a first predetermined cutoff, wherein, when the reversal score is greater than or less than the first predetermined cutoff, the risk score is calculated from a comparison of a reversal value of a second biomarker pair from the panel to a second predetermined cutoff value.

In some embodiments, this disclosure provides a method of treating or preventing placental dysfunction in a pregnant female, wherein calculating the risk score further comprises, when the reversal score of the second biomarker pair is greater than or less than the second predetermined cutoff, the risk score is calculated from a comparison of a reversal value of a third biomarker pair from the panel to a third predetermined cutoff value.

In some embodiments, this disclosure provides a method of treating or preventing placental dysfunction in a pregnant female, wherein the risk score is assigned a value based on whether the reversal value of the third biomarker pair is greater than or less than the third predetermined cutoff value.

In some embodiments, this disclosure provides a method of treating or preventing placental dysfunction in a pregnant female, wherein calculating the risk score comprises comparing a reversal value of a first biomarker pair to a first predetermined cutoff and, when the reversal score is greater than or less than the first predetermined cutoff, the risk score is assigned a value.

In some embodiments, this disclosure provides a method of treating or preventing placental dysfunction in a pregnant female, wherein calculating the risk score comprises comparing the reversal value of the second biomarker pair to the second predetermined cutoff and, when the reversal score is greater than or less than the second predetermined cutoff, the risk score is assigned a value.

In another aspect, this disclosure provides a method for treating or preventing placental dysfunction in a pregnant female, the method comprising: (a) detecting the panel of isolated nucleic acid biomarkers of any one of the panels listed in Table 6 in a biological sample obtained from a pregnant female; (b) providing a risk score based upon measured levels of the labeled and/or amplified nucleic acid molecules; (c) determining that said pregnant female has an increased likelihood of placental dysfunction based at least in part on said risk score exceeding a reference score; and (d) recommending, facilitating, prescribing, or administering one or more therapies to the pregnant female based on the risk score, to treat or prevent placental dysfunction.

In some embodiments, this disclosure provides a method of treating or preventing placental dysfunction in a pregnant female, wherein the nucleic acid biomarkers are normalized. In some embodiments, normalization comprises a reversal of the isolated nucleic acid biomarkers.

In some embodiments, this disclosure provides a method of treating or preventing placental dysfunction in a pregnant female, wherein the reversal comprises an upregulated biomarker and a downregulated biomarker.

In some embodiments, this disclosure provides a method of treating or preventing placental dysfunction in a pregnant female, wherein the risk score is calculated based on the reversal of data values.

In some embodiments, this disclosure provides a method of treating or preventing placental dysfunction in a pregnant female, wherein the method further comprises a step of determining gestational age at blood draw (GABD).

In some embodiments, this disclosure provides a method of treating or preventing placental dysfunction in a pregnant female, wherein determining GABD is performed before the detecting step.

In some embodiments, this disclosure provides a method of treating or preventing placental dysfunction in a pregnant female, wherein GABD is between 119-153 days, 154-196 days, and/or 119-202 days.

In some embodiments, this disclosure provides a method of treating or preventing placental dysfunction in a pregnant female, wherein the method determines a pregnant female's risk of developing placental dysfunction across the full window of gestation.

In some embodiments, this disclosure provides a method of treating or preventing placental dysfunction in a pregnant female, wherein the full gestation window of gestation is between 119-202 days of gestation.

In some embodiments, this disclosure provides a method of treating or preventing placental dysfunction in a pregnant female, wherein the method determines a pregnant female's risk of developing placental dysfunction early in the pregnancy. In some embodiments, early pregnancy is between 119-153 days of gestation.

In some embodiments, this disclosure provides a method of treating or preventing placental dysfunction in a pregnant female, wherein the method determines a pregnant female's risk of developing placental dysfunction later in the pregnancy. In some embodiments, late pregnancy is between 154-196 days of gestation.

In some embodiments, this disclosure provides a method of treating or preventing placental dysfunction in a pregnant female, wherein the placental dysfunction is preeclampsia.

In some embodiments, this disclosure provides a method of treating or preventing placental dysfunction in a pregnant female, wherein calculating the risk score is performed using a binary decision tree analysis.

In some embodiments, this disclosure provides a method of treating or preventing placental dysfunction in a pregnant female, wherein the binary decision tree analysis comprises comparing reversal values of two or more biomarker pairs to two or more predetermined cutoff values and assigning the risk score based on the results of said comparison.

In some embodiments, this disclosure provides a method of treating or preventing placental dysfunction in a pregnant female, wherein calculating the risk score comprises comparing a reversal value of a first biomarker pair from the panel to a first predetermined cutoff, wherein, when the reversal score is greater than or less than the first predetermined cutoff, the risk score is calculated from a comparison of a reversal value of a second biomarker pair from the panel to a second predetermined cutoff value.

In some embodiments, this disclosure provides a method of treating or preventing placental dysfunction in a pregnant female, wherein calculating the risk score further comprises, when the reversal score of the second biomarker pair is greater than or less than the second predetermined cutoff, the risk score is calculated from a comparison of a reversal value of a third biomarker pair from the panel to a third predetermined cutoff value.

In some embodiments, this disclosure provides a method of treating or preventing placental dysfunction in a pregnant female, wherein the risk score is assigned a value based on whether the reversal value of the third biomarker pair is greater than or less than the third predetermined cutoff value.

In some embodiments, calculating the risk score comprises comparing a reversal value of a first biomarker pair to a first predetermined cutoff and, when the reversal score is greater than or less than the first predetermined cutoff, the risk score is assigned a value.

In some embodiments, calculating the risk score comprises comparing the reversal value of the second biomarker pair to the second predetermined cutoff and, when the reversal score is greater than or less than the second predetermined cutoff, the risk score is assigned a value.

Other features and advantages of the invention will be apparent from the detailed description, and from the claims.

### BRIEF DESCRIPTION OF DRAWINGS

FIG. 1 shows an exemplary binary decision tree for determining risk of placental dysfunction in which terminal nodes have been annotated with risk scores. The binary decision tree is modeled using the panel of biomarkers identified as “panel 1” from the list of nucleic acid biomarker pairs listed in Table 6.

FIG. 2 shows an exemplary binary decision tree for determining risk of placental dysfunction in which terminal nodes have been annotated with risk scores. The binary decision tree is modeled using the panel of biomarkers identified as “panel 5” from the list of nucleic acid biomarker pairs listed in Table 6.

FIG. 3 shows an exemplary binary decision tree for determining risk of placental dysfunction in which terminal nodes have been annotated with risk scores. The binary decision tree is modeled using the panel of biomarkers identified as “panel 31” from the list of nucleic acid biomarker pairs listed in Table 6.

FIG. 4 shows an exemplary binary decision tree for determining risk of placental dysfunction in which terminal nodes have been annotated with risk scores. The binary decision tree is modeled using the panel of biomarkers identified as “panel 50” from the list of nucleic acid biomarker pairs listed in Table 6.

### DETAILED DESCRIPTION

The present disclosure is based, generally, on the discovery that the concentration of certain extracellular microRNA (miRNA) biomarkers present in the maternal circulation during pregnancy predicts subsequent risk of developing placental dysfunction later in the pregnancy. For each of the miRNA biomarkers disclosed herein, the concentration of miRNA in the maternal circulation is altered in women who subsequently develop placental dysfunction. Advantageously, expression levels of these miRNA biomarkers can be measured from blood samples, thereby providing a minimally-invasive means for prediction of placental dysfunction, which can manifest as preeclampsia, intrauterine growth restriction, preterm birth (PTB), preterm labor, preterm premature rupture of membranes, late spontaneous abortion and abruption placentae.

The present disclosure is further specifically based, in part, on the unexpected discovery that single-miRNA biomarker and pairs of miRNA biomarkers disclosed herein can be utilized in methods of predicting a pregnant female's risk of developing placental dysfunction later in the pregnancy. Each of the miRNA biomarkers and clinical variables disclosed herein, either alone or as components of pairs, ratios and/or reversal pairs serve as biomarkers for determining a pregnant women's risk of developing placental dysfunction later in the pregnancy.

A reversal value is the ratio of the abundance of an up regulated biomarker over a down regulated biomarker and serves to both normalize variability and amplify diagnostic signal. The invention lies, in part, in the selection of particular biomarkers that, when paired together, can accurately determine a pregnant female's risk of developing placental dysfunction later in the pregnancy. Accordingly, it is human ingenuity in selecting the specific biomarkers that are informative upon being paired, for example, in novel reversals, and/or the data transformations, for example the ratio of log values, in forming said reversals, that underlies the present invention.

The disclosure provides single-miRNA biomarkers and pairs of miRNA biomarkers as well as associated panels, methods and kits for determining a pregnant female's risk of developing placental dysfunction later in the pregnancy.

In addition to the specific miRNA biomarkers identified in this disclosure, for example, by name, sequence, or reference, the invention also contemplates use of biomarker variants that are at least 90% or at least 95% or at least 97% identical to the exemplified sequences and that are now known or later discovered and that have utility for the methods of the invention. These variants may represent polymorphisms, splice variants, mutations, and the like. In this regard, the instant specification discloses multiple art-known analytes in the context of the invention and provides exemplary peptide sequences that can be used to identify these analytes. However, those skilled in the art appreciate that additional sequences or other information can easily be identified that can provide additional characteristics of the disclosed biomarkers and that the exemplified references are in no way limiting with regard to the disclosed nucleic acid.

As described herein, various techniques and reagents find use in the methods of the present invention. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As described herein, nucleic acid can be detected through a variety of assays and techniques known in the art.

The miRNA biomarkers that can be components of reversal pairs described herein include, for example, the miRNA biomarkers listed in Table 4.

In some embodiments, the invention provides a method of determining a pregnant female's risk of developing placental dysfunction later in the pregnancy, the method comprising measuring in a biological sample obtained from the pregnant female a reversal value for one or more of the biomarker pairs listed in Table 4.

In some embodiments, the invention provides a method of determining a pregnant female's risk of developing placental dysfunction later in the pregnancy, the method comprising measuring in a biological sample obtained from the pregnant female a reversal value for one pair of biomarkers selected from the biomarker pairs listed in Table 4.

In some embodiments, the invention provides a pair of isolated biomarkers selected from the biomarker pairs listed in Table 4, wherein the pair of biomarkers exhibits a higher ratio in pregnant females that will develop placental dysfunction later in the pregnancy relative to pregnant females that will not develop placental dysfunction.

In one embodiment, the present invention provides a composition comprising a pair of isolated biomarkers selected from the group consisting of the biomarker pairs listed in Table 4, wherein the pair of biomarkers exhibits a higher ratio in pregnant females that will develop placental dysfunction later in the pregnancy relative to pregnant females that will not develop placental dysfunction.

In one embodiment, the biological sample is selected from the group consisting of whole blood, plasma, and serum. In one embodiment, the biological sample is serum. In some embodiments, the sample is obtained between 17 and 28 weeks of gestational age at blood draw (GABD). In some embodiments, the samples is obtained between 17 and 29 weeks. In some embodiments, the sample is obtained between 19 and 27 weeks. In some embodiments, the sample is obtained between 18 and 21 weeks. In further embodiments, the sample is obtained between 23 and 28 weeks of GABD. In some embodiments, the sample is obtained between 18 and 28 weeks of GABD. In some embodiments, the sample is obtained between 18 and 36 weeks of GABD. In further embodiments the sample is obtained between 19 and 21 weeks of GABD. In some embodiments, the sample is obtained between 20 and 22 weeks of GABD. In some embodiments, the sample is obtained between 21 and 23 weeks of GABD. In further embodiments, the sample is obtained between 22 and 24 weeks of GABD. In additional embodiments, the sample is obtained between 23 and 25 weeks of GABD. In some embodiments, the sample is obtained between 24 and 26 weeks of GABD. In further embodiments, the sample is obtained between 25 and 27 weeks of GABD. In additional embodiments, the sample is obtained between 26 and 28 weeks of GABD. In some embodiments, the sample is obtained between 27 and 29 weeks of GABD. In further embodiments, the sample is obtained between 28 and 30 weeks of GABD. In additional embodiments, the sample is obtained between 29 and 31 weeks of GABD. In some embodiments, the sample is obtained between 30 and 32 weeks of GABD. In further embodiments, the sample is obtained between 31 and 33 weeks of GABD. In additional embodiments, the sample is obtained between 32 and 34 weeks of GABD. In some embodiments, the sample is obtained between 33 and 35 weeks of GABD. In further embodiments, the sample is obtained between 34 and 36 weeks of GABD. In additional embodiments, the sample is obtained between 17 and 36 weeks. In further embodiments, the sample is obtained more than 17, more than 18, more than 19, more than 20, more than 21, more than 22, more than 23, more than 24, more than 25, more than 26, more than 27, more than 28, more than 29, more than 30, more than 31, more than 32, more than 33, more than 34 or more than 35 weeks of GABD.

In some embodiments, the sample is obtained between 119 and 202 days of GABD. In further embodiments, the sample is obtained between 119 and 152 days of GABD. In some embodiments, the sample is obtained between 119 and 153 days of GABD. In some embodiments, the sample is obtained between 138 and 172 days of GABD. In further embodiments, the sample is obtained between 154 and 196 days of GABD. In other embodiments, the samples is obtained between 156 and 196 days of GABD. In additional embodiments, the sample is obtained between 119 and 252 days. In further embodiments, the sample is obtained more than 119, more than 126, more than 133, more than 140, more than 147, more than 154, more than 161, more than 168, more than 175, more than 182, more than 189, more than 196, more than 203, more than 210, more than 217, more than 224, more than 231, more than 238, or more than 245 days of GABD.

In addition to the specific biomarkers, the disclosure further includes biomarker variants that are about 90%, about 95%, or about 97% identical to the exemplified sequences. Variants, as used herein, include polymorphisms, splice variants, mutations, and the like. Although described with reference to nucleic acid biomarkers, changes in reversal value can be identified at the gene expression level or protein level for pairs of biomarkers.

The compositions and methods of the invention also can include clinical variables, including but not limited to, maternal characteristics, medical history, past pregnancy history, and obstetrical history. Such additional clinical variables can include, for example, previous low birth weight or preterm delivery, multiple 2nd trimester spontaneous abortions, prior first trimester induced abortion, familial and intergenerational factors, history of infertility, parity, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight, low or high body mass index, diabetes, diabetes mellitus, chronic diabetes, chronic diabetes mellitus, chronic hypertension, urogenital infections (i.e. urinary tract infection), asthma, anxiety and depression, asthma, hypertension, hypothyroidism, high body mass index (BMI), low BMI, BMI. Demographic risk indicia for preterm birth can include, for example, maternal age, race/ethnicity, single marital status, low socioeconomic status, maternal age, employment-related physical activity, occupational exposures and environment exposures and stress. Further clinical variables can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman RE, Butler AS, editors. Washington (DC): National Academies Press (US); 2007). Additional clinical variables useful for as markers can be identified using learning algorithms known in the art, such as linear discriminant analysis, support vector machine classification, recursive feature elimination, prediction analysis of microarray, logistic regression, CART, Flex Tree, LART, random forest, MART, and/or survival analysis regression, which are known to those of skill in the art and are further described herein.

The present disclosure describes and exemplifies various models and corresponding biomarkers that perform at high levels of accuracy and precision in determining a pregnant female's risk of developing placental dysfunction later in the pregnancy. In some embodiments, the markers disclosed herein are predictive across the entire GABD range. In some embodiments, the entire GABD range represents from 17 to 36 weeks. In further embodiments, the entire GABD range represents more than 17, more than 18, more than 19, more than 20, more than 21, more than 22, more than 23, more than 24, more than 25, more than 26, more than 27, more than 28, more than 29, more than 30, more than 31, more than 32, more than 33, more than 34 or more than 35 weeks. In additional embodiments, the entire GABD range represents more than 119, more than 126, more than 133, more than 140, more than 147, more than 154, more than 161, more than 168, more than 175, more than 182, more than 189, more than 196, more than 203, more than 210, more than 217, more than 224, more than 231, more than 238, or more than 245 days of GABD.

It will be understood by those of skill in the art, that other models are known in the art that can be used to practice the claimed inventions and that the performance of a model can be evaluated in a variety of ways, including, but not limited to accuracy, precision, recall/sensitivity, weighted average of precision and recall. Models known in the art include, without limitation, linear discriminant analysis, support vector machine classification, recursive feature elimination, prediction analysis of microarray, logistic regression, CART, FlexTree, LART, random forest, MART, and/or survival analysis regression.

In some embodiments, performance of a model can be evaluated based on accuracy. For example, accuracy can be expressed as the percentage of time, for example, 50%, 51%, 52%, 53%, 54%, 55%, 56%, 57%, 58%, 59%, 60%, 61%, 62%, 63%, 64%, 65%, 70%, 71%, 72%, 73%, 74%, 75%, 76%, 77%, 80% or more that a model accurately predicts a pregnant female's risk of developing placental dysfunction later in the pregnancy.

The present disclosure is based in part on the surprising discovery that the selection of certain biomarkers and/or clinical variables enables determining a pregnant female's risk of developing placental dysfunction later in the pregnancy.

It must be noted that, as used in this specification and the appended claims, the singular forms “a”, “an” and “the” include plural referents unless the content clearly dictates otherwise and are used interchangeably with “at least one” and “one or more.” Thus, for example, reference to “a biomarker” includes a mixture of two or more biomarkers, and the like.

The term “about,” particularly in reference to a given quantity, is meant to encompass deviations of plus or minus five percent.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

As used herein, the term “panel” refers to a composition, such as an array or a collection, comprising one or more biomarkers. The term can also refer to a profile or index of expression patterns of one or more biomarkers described herein. The number of biomarkers useful for a biomarker panel is based on the sensitivity and specificity value for the particular combination of biomarker values. The term can also refer to a profile or index of expression patterns of two or more biomarkers described herein. The number of biomarkers useful for a biomarker panel can, in some embodiments, be based on the sensitivity and specificity value for the particular combination of biomarker values. Biomarkers in a panel, or the panel as a unit, can be combined in some embodiments of the invention with other markers, including other biomarkers and/or clinical or demographic variables (e.g., BMI, GABD). Such combinations can be used to derive combined scores predictive of, and which can in turn be used to treat, pregnancy dysfunction.

As used herein, and unless otherwise specified, the terms “isolated” and “purified” generally describes a composition of matter that has been removed from its native environment (e.g., the natural environment if it is naturally occurring), and thus is altered by the hand of man from its natural state so as to possess markedly different characteristics with regard to at least one of structure, function and properties. An isolated protein or nucleic acid is distinct from the way it exists in nature and includes synthetic nucleic acids, peptides and proteins.

The term “biomarker” refers to a biological molecule, a fragment of a biological molecule, or a clinical variable the change and/or the detection of which can be correlated with a particular physical condition or state. The terms “marker” and “biomarker” are used interchangeably throughout the disclosure. For example, the biomarkers of the present invention are associated with a discrimination power between pregnant females that will develop placental dysfunction later in the pregnancy versus those that will not develop placental dysfunction. Such biomarkers include any suitable analyte, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins). The term also encompasses miRNAs and portions or fragments of a miRNAs.

As used herein, the term “reversal” refers to the ratio of the measured value of one or more analytes (or some conversion or representation thereof) over that of one or more analytes, wherein the analytes provide some partially independent information. In some embodiments, the analyte value (or its representation) is itself a normalized value of the measured endogenous analyte relative to, e.g., values in the same processing batch referred to herein as: response ratio or relative ratio. For example, as exemplified herein, hsa-miR-516-5p/hsa-miR-485-5p is a ratio of an up-regulated analyte in the numerator and a down-regulated analyte in the denominator, which is defined herein as a “reversal pair.” A ratio of two up-regulated analytes in the numerator and one down-regulated analyte in the denominator, which is within the definition of a “reversal triplet” defined herein. In some embodiments, “/” is inclusive of but not limited to division, taking a root or difference. In other embodiments, “+” is inclusive of but not limited to addition, taking a power or multiplication. In all instances, one analyte in a reversal may serve to normalize another analyte (e.g. decrease variability due to biomedical conditions not of interest, or sample pre-analytical or analytical variability). In the particular case of a ratio that is a “reversal” both amplification of diagnostic signal and normalization are possible. It is understood, that the methods of the invention are not limited to the subset of reversals, but also encompass ratios of biomarkers.

As used herein, the term “reversal pair” refers to biomarkers in pairs where a mathematical combination of the values of the pair exhibits a change in value between the classes being compared. The detection of reversals in analyte (i.e. miRNA) concentrations eliminates the need for data normalization or the establishment of population-wide thresholds, and requires only that the levels of two or more biomarkers change relative to each other. Encompassed within the definition of any reversal pair is the corresponding reversal pair wherein individual biomarkers are switched between the numerator and denominator. One skilled in the art will appreciate that such a corresponding reversal pair is equally informative with regard to its predictive power.

The term “reversal value” refers to the ratio of the abundance of two analytes and serves to both normalize variability and amplify diagnostic signal. In some embodiments, a reversal value refers to the ratio of the abundance of an up-regulated (interchangeably referred to as “over-abundant,” up-regulation as used herein simply refers to an observation of abundance) analyte over a down-regulated analyte (interchangeably referred to as “under-abundant,” down-regulation as used herein simply refers to an observation of relative abundance). In some embodiments, a reversal value refers to the ratio of an up-regulated analyte over an up-regulated analyte, where one analyte differs in the degree of up-regulation relative the other analyte. In some embodiments, a reversal value refers to the ratio a down-regulated analyte over a down-regulated analyte, where one analyte differs in the degree of down-regulation relative the other analyte. In some embodiments a reversal value refers to the ratio of a regulated analyte (up or down) and an analyte that is un-regulated. In this case the un-regulated analyte can still serve to normalize. In some embodiments, a reversal value refers to the ratio of two analytes that are un-regulated or whose directions of regulation are unknown. In this case, the un-regulated analytes can still serve to normalize each other and to reveal a diagnostic signal.

One advantageous aspect of a reversal is the presence of complementary information in the two analytes, so that the combination of the two is more diagnostic of the condition of interest than either one alone. Preferably the combination of the two analytes increases signal-to-noise ratio by compensating for biomedical conditions not of interest, pre-analytic variability and/or analytic variability. Out of all the possible reversals within a narrow window, a subset can be selected based on individual univariate performance. Additionally, a subset can be selected based on bivariate or multivariate performance in a training set, with testing on held-out data or on bootstrap iterations. For example, logistic or linear regression models can be trained, optionally with parameter shrinkage by L1 or L2 or other penalties, and tested in leave-one-out, leave-pair-out or leave-fold-out cross-validation, or in bootstrap sampling with replacement, or in a held-out data set. As disclosed herein, the ratio of the abundance of two analytes, for example, the ratio of an up-regulated biomarker over a down-regulated biomarker, referred herein as a reversal value, can be used to identify robust and accurate classifiers and predict a pregnant female's risk of developing placental dysfunction later in the pregnancy

Use of a ratio of biomarkers in the methods disclosed herein corrects for variability that is the result of human manipulation after the removal of the biological sample from the pregnant female. Such variability can be introduced, for example, during sample collection, processing, depletion, digestion or any other step of the methods used to measure the biomarkers present in a sample and is independent of how the biomarkers behave in nature. Accordingly, the invention generally encompasses the use of a reversal pair in a method of diagnosis or prognosis to reduce variability and/or amplify, normalize or clarify diagnostic signal.

While the term reversal value can refer to the ratio of the abundance of an up regulated analyte over a down regulated analyte and serves to both normalize variability and amplify diagnostic signal, it is also contemplated that a pair of biomarkers of the invention could be treated in a classifier by any other means, for example, by subtraction, addition or multiplication of abundances. In addition, it is contemplated that a value can be mathematically converted to a different value and used to determine a ratio. For example, as disclosed herein, reversals can be constructed as the ratios of the logarithm (log) values. Similarly, ratios can be mathematically converted, for example, as the log of the ratioed values. The methods disclosed herein encompass the measurement of biomarker pairs by such other means. A person skilled in the art will readily understand suitable data transformations that can be applied to identify biomarkers predictive of placental dysfunction, including the data transformations disclosed herein. Exemplary transformations include, but are not limited to, box-cox, root, inverse, rank and log. Such data transformations are well known in the art, for example, root (where the root transformation is selected as appropriate for the data set, such as 2, 3, 4, and higher, as appropriate), inverse (1/X), rank (assigning to an ordered list based on appropriate criteria), and so forth, as is well known in the art.

This method is advantageous because it provides the simplest possible classifier that may be independent of data normalization, helps to avoid overfitting, and results in a very simple experimental test that is easy to implement in the clinic. In some uses of the term “reversal” it refers to the identification of analyte pairs where the relative expression (rank order) of each member of a pair reverses in the two conditions studied (e.g. cancer vs not cancer, placental dysfunction vs not). Reversal, as it is used here, allows for there to be opposing regulation of the two members of the pair (e.g., up or down), but does not require that their rank order in abundance to “reverse” in the different clinical conditions. The use of marker pairs based on changes in reversal values that are independent of data normalization enabled the development of the clinically relevant biomarkers disclosed herein. Because quantification of any single analyte is subject to uncertainties caused by measurement variability, normal fluctuations, and individual related variation in baseline expression, identification of pairs of markers that may be under coordinated, systematic regulation enables robust methods for diagnosis and prognosis.

While the specification discloses embodiments directed to measuring the particular pairs of biomarkers disclosed in Table 4, the invention is not restricted to the particular pairs recited in Table 4 and individual biomarkers disclosed herein, for example, in Table 3, as well as any pair or panel of the individual biomarkers is also encompassed by the present invention, as are methods comprising one or more pairs of biomarkers, for example, pairs of biomarkers comprising the biomarkers of Tables 3, 4 or 5. It is understood that the univariate and bivariate biomarkers disclosed herein, for example, in Tables 3, 4 or 5, can be used as biomarkers, either singly, in combinations of 2 or more biomarkers, as panels, or in combination with other variables (for example, proteins, metabolites, other molecules, clinical factors, and/or demographic factors) to predict placental dysfunction, such as preeclampsia, as disclosed herein. A person skilled in the art can readily contemplate these and/or additional parameters that can be combined with the biomarkers disclosed herein to predict placental dysfunction.

In addition to biomarkers, measurable features can further include clinical variables including, for example, maternal characteristics, age, race, ethnicity, medical history, past pregnancy history, obstetrical history. For a risk indicium, a measurable feature can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight/low body mass index, diabetes, hypertension, urogenital infections, hypothyroidism, asthma, low educational attainment, cigarette smoking, drug use and alcohol consumption, prior miscarriage, prior stillbirth, Body Mass Index (BMI), maternal age, parity, gravidity, fetal gender, height and weight separately from BMI, race and socioeconomic status. Additional markers can include, for example, previous low birth weight or preterm delivery, multiple 2nd trimester spontaneous abortions, prior first trimester induced abortion, familial and intergenerational factors, history of infertility, nulliparity, gravidity, primigravida, multigravida, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight, low or high body mass index, diabetes, hypertension, urogenital infections (i.e. urinary tract infection), asthma, anxiety and depression, asthma, hypertension, hypothyroidism, low educational attainment, drug use and alcohol consumption. Demographic risk indicia for preterm birth can include, for example, maternal age, race/ethnicity, single marital status, low socioeconomic status, maternal age, employment-related physical activity, occupational exposures and environment exposures and stress. Further risk indicia can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman RE, Butler AS, editors. Washington (DC): National Academies Press (US); 2007). Additional risk indicia useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

In some embodiments, the methods of the invention comprise calculation of body mass index (BMI).

As used herein, the term “risk score” refers to a score that can be assigned based on comparing the amount of one or more biomarkers or reversal values in a biological sample obtained from a pregnant female to a standard or reference score that represents an average amount of the one or more biomarkers calculated from biological samples obtained from a random pool of pregnant females. Alternatively, the calculated “risk score” can be compared to the average population risk (prevalence of the outcomes). A “risk score” can also be calculated using a binary decision tree analysis. For example, a “risk score” can be assigned to an individual subject assigned to a particular node of a binary decision tree based on the proportion of cases amongst all subjects assigned to that node in the clinical validation set, as described in Example 4. As will be apparent to one of skill in the art, a risk score can represent the positive predictive value (PPV) of the pregnant female's one or more biomarkers or reversal values for occurrence of the event, i.e., placental dysfunction. A risk score can also represent the probability of occurrence of the event given the pregnant female's one or more biomarkers or reversal values. In a simple embodiment, the pregnant female's risk prior to measurement of biomarkers (pre-test risk) is assigned to be the average population risk (prevalence of the event). Her risk is updated upon measurement of biomarkers and to a post-test risk by calculation of the risk score. An individual pre-test risk can also be assigned to a pregnant female based her standard clinical and demographic data, or on individual, family or ancestral health history or genetic data. For example, a pregnant female with a history of prior preeclampsia may have a greater individual risk for placental dysfunction than the average population risk. The calculated risk based on biomarkers can then be an updated (post-test) risk for the current pregnancy, beyond that individual pre-test risk. A calculated risk of placental dysfunction can also be updated by events or information gathered after the test is applied in the current pregnancy. For example, a pregnant female with a calculated risk of placental dysfunction of 30%, but exhibiting later signs or symptoms (e.g., moderately elevated blood pressure) may have an even higher risk of placental dysfunction (>30%) given the combination of the test and the later sign or symptom. In some embodiments, the risk score is expressed as the log of the reversal value, i.e. the ratio of the relative intensities of the individual biomarkers. One skilled in the art will appreciate that a risk score can be expressed based on a various data transformations as well as being expressed as the ratio itself. Furthermore, with particular regard to reversal pairs, one skilled in the art will appreciate that any ratio is equally informative if the biomarkers in the numerator and denominator are switched or that related data transformations (e.g., subtraction) are applied. Because the level of a biomarker may not be static throughout pregnancy, a standard or reference score has to have been obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more biomarkers calculated from biological samples obtained from a random pool of pregnant females.

In some embodiments, the methods of the invention can be practiced with samples obtained from pregnant females with a specified BMI. Briefly, BMI is an individual's weight in kilograms divided by the square of height in meters. BMI does not measure body fat directly, but research has shown that BMI is correlated with more direct measures of body fat obtained from skinfold thickness measurements, bioelectrical impedance, densitometry (underwater weighing), dual energy x-ray absorptiometry (DXA) and other methods. Furthermore, BMI appears to be as strongly correlated with various metabolic and disease outcome as are these more direct measures of body fatness. Generally, an individual with a BMI below 18.5 is considered underweight, an individual with a BMI of equal or greater than 18.5 to 24.9 normal weight, while an individual with a BMI of equal or greater than 25.0 to 29.9 is considered overweight and an individual with a BMI of equal or greater than 30.0 is considered obese. In some embodiments, the predictive performance of the claimed methods can be improved with a BMI stratification of equal or greater than 18, equal or greater than 19, equal or greater than 20, equal or greater than 21, equal or greater than 22, equal or greater than 23, equal or greater than 24, equal or greater than 25, equal or greater than 26, equal or greater than 27, equal or greater than 28, equal or greater than 29 or equal or greater than 30. In other embodiments, the predictive performance of the claimed methods can be improved with a BMI stratification of equal or less than 18, equal or less than 19, equal or less than 20, equal or less than 21, equal or less than 22, equal or less than 23, equal or less than 24, equal or less than 25, equal or less than 26, equal or less than 27, equal or less than 28, equal or less than 29 or equal or less than 30.

In the context of the present invention, the term “biological sample,” encompasses any sample that is taken from pregnant female, and particularly from a pregnant human, and contains one or more of the biomarkers disclosed herein. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

The term “amount” or “level” as used herein refers to a quantity of a biomarker that is detectable or measurable in a biological sample and/or control. The quantity of a biomarker can be, for example, the quantity of nucleic acid (i.e. miRNA), the quantity of a polypeptide, the quantity of nucleic acid, or the quantity of a fragment or surrogate. The term can alternatively include combinations thereof. The term “amount” or “level” of a biomarker is a measurable feature of that biomarker.

In some embodiments, nucleic acid amplification methods can be used to detect a polynucleotide biomarker. For example, the oligonucleotide primers and probes of the present invention can be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and subsequent updates)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254:1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315), digital PCR (see, e.g., Salipante et al., Clin. Chem. doi: 10.1373/clinchem.2019.304048 (2019)), and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; US Publication No. 2006/46265.

In some embodiments, measuring mRNA in a biological sample can be used as a surrogate for detection of the level of the corresponding protein biomarker in a biological sample. Thus, any of the biomarkers, biomarker pairs or biomarker reversal panels described herein can also be detected by detecting the appropriate RNA. Levels of mRNA can measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Digital PCR is a special case of qPCR, where PCR is performed in many discrete partitions of the sample, and can be more sensitive and reliable than traditional qPCR (see, e.g., Salipante et al., Clin. Chem. doi: 10.1373/clinchem.2019.304048 (2019)). Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

In one aspect, the invention provides a panel of isolated nucleic acid biomarkers comprising two or more of the nucleic acid biomarkers listed in Tables 3, 4 or 5. In another aspect, the invention provides a panel of isolated nucleic acid biomarkers selected from one or more of the panels of nucleic acid biomarkers listed in Table 6. In another aspect, the invention provides a panel of isolated nucleic acid biomarkers selected from one or more of the panels of nucleic acid biomarkers listed in Table 6 in combination with at least one of the nucleic acid biomarkers listed in any one of Tables 3, 4, or 5.

In a further aspect, the invention provides a panel of isolated nucleic acid biomarkers comprising two or more of the nucleic acid biomarkers listed in Table 3.

In some embodiments, a panel described herein comprises hsa-miR-516b-5p. In some embodiments, a panel described herein comprises hsa-miR-30c-5p. In some embodiments, a panel described herein comprises hsa-miR-1-3p. In some embodiments, a panel described herein comprises hsa-miR-331-3p. In some embodiments, a panel described herein comprises hsa-miR-485-5p. In some embodiments, a panel described herein comprises hsa-miR-181d-5p. In some embodiments, a panel described herein comprises hsa-miR-23a-3p. In some embodiments, a panel described herein comprises hsa-miR-378d. In some embodiments, a panel described herein comprises hsa-miR-155-5p. In some embodiments, a panel described herein comprises hsa-miR-99b-3p. In some embodiments, a panel described herein comprises hsa-miR-1249-3p. In some embodiments, a panel described herein comprises hsa-miR-142-3p. In some embodiments, a panel described herein comprises hsa-miR-4433b-5p. In some embodiments, a panel described herein comprises hsa-miR-1273h-3p. In some embodiments, a panel described herein comprises hsa-miR-18a-3p. In some embodiments, a panel described herein comprises hsa-miR-4746-5p. In some embodiments, a panel described herein comprises hsa-miR-27a-5p. In some embodiments, a panel described herein comprises hsa-miR-654-3p. In some embodiments, a panel described herein comprises hsa-miR-7-5p. In some embodiments, a panel described herein comprises hsa-miR-941.

In some embodiments, a panel described herein comprises hsa-miR-516b-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-30c-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-1-3p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-516b-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-30c-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-1-3p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In one aspect, the invention provides a biomarker panel comprising two or more of the isolated nucleic acid biomarkers selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, the invention provides a biomarker panel comprising hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, the invention provides a biomarker panel comprising hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, the invention provides a biomarker panel comprising hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, the invention provides a biomarker panel comprising hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, the invention provides a biomarker panel comprising hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, the invention provides a biomarker panel comprising hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, the invention provides a biomarker panel comprising hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, the invention provides a biomarker panel comprising hsa-miR-7-5p/hsa-miR-485-5p; hsa-miR-1306-3p/hsa-miR-664a-5p; and hsa-miR-501-3p/hsa-miR-484.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, the invention provides a biomarker panel comprising hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, the invention provides a biomarker panel comprising hsa-miR-7-5p/hsa-miR-485-5p; hsa-miR-1306-3p/hsa-miR-664a-5p; and hsa-miR-628-3p/hsa-miR-19b-3p.

In some embodiments, the invention provides a biomarker panel comprising hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, the invention provides a biomarker panel comprising hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, the invention provides a biomarker panel comprising hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, the invention provides a biomarker panel comprising hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p.

In some embodiments, the invention provides a biomarker panel comprising hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, the invention provides a biomarker panel comprising hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, the invention provides a biomarker panel comprising hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, the invention provides a biomarker panel comprising hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, the invention provides a biomarker panel comprising hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, the invention provides a biomarker panel comprising hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, the invention provides a biomarker panel comprising hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, the invention provides a biomarker panel comprising hsa-miR-7-5p/hsa-miR-485-5p; hsa-miR-30c-5p/hsa-miR-125a-5p; and hsa-miR-1-3p/hsa-miR-345-5p.

In some embodiments, the invention provides a biomarker panel comprising hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, the invention provides a biomarker panel comprising hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, the invention provides a biomarker panel comprising hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p.

In some embodiments, the invention provides a biomarker panel comprising hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, the invention provides a biomarker panel comprising hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, the invention provides a biomarker panel comprising hsa-miR-7-5p/hsa-miR-485-5p; hsa-miR-1260b/hsa-mir-320c-2; and hsa-miR-433-3p/hsa-miR-345-5p.

In some embodiments, the invention provides a biomarker panel comprising hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, the invention provides a biomarker panel comprising hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, the invention provides a biomarker panel comprising hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, the invention provides a biomarker panel comprising hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, the invention provides a biomarker panel comprising hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, the invention provides a biomarker panel comprising hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, the invention provides a biomarker panel comprising hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, the invention provides a biomarker panel comprising hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, the invention provides a biomarker panel comprising hsa-miR-7-5p/hsa-miR-485-5p; hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, the invention provides a biomarker panel comprising hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, the invention provides a biomarker panel comprising hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, the invention provides a biomarker panel comprising hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, the invention provides a biomarker panel comprising hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, the invention provides a biomarker panel comprising hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, the invention provides a biomarker panel comprising hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, the invention provides a biomarker panel comprising hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, the invention provides a biomarker panel comprising hsa-miR-7-5p/hsa-miR-485-5p; hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, the invention provides a biomarker panel comprising hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a biomarker panel comprising hsa-miR-7-5p/hsa-miR-485-5p; hsa-miR-221-5p/hsa-miR-760; hsa-miR-99b-3p/hsa-miR-340-5p; and hsa-miR-1306-3p/hsa-miR-150-3p.

In some embodiments, the invention provides a biomarker panel comprising hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, the invention provides a biomarker panel comprising hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, the invention provides a biomarker panel comprising hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a biomarker panel comprising hsa-miR-330-5p/hsa-miR-223-3p; hsa-miR-7-5p/hsa-miR-485-5p; and hsa-miR-511-5p/hsa-miR-148a-5p.

In some embodiments, the invention provides a biomarker panel comprising hsa-miR-7-5p/hsa-miR-485-5p; hsa-miR-1306-3p/hsa-miR-664a-5p; hsa-miR-30a-5p/hsa-miR-941; and hsa-miR-30c-5p/hsa-miR-27b-3p.

In some embodiments, the biomarker panel comprises isolated nucleic acid biomarkers comprising hsa-miR-1-3p, hsa-miR-181d-5p, hsa-miR-378d, hsa-miR-4433b-5p, and/or hsa-miR-27a-5p. In some embodiments, the biomarker panel comprises isolated nucleic acid biomarkers comprising hsa-miR-516b-5p and/or hsa-miR-30c-5p. In some embodiments, the biomarker panel comprises isolated nucleic acid biomarkers comprising hsa-miR-516b-5p, hsa-miR-30c-5p, hsa-miR-1-3p, and/or hsa-miR-331-3p. In some embodiments, the biomarker panel comprises isolated nucleic acid biomarkers comprising hsa-miR-516b-5p, hsa-miR-30c-5p, hsa-miR-1-3p, hsa-miR-331-3p, hsa-miR-485-5p, and/or hsa-miR-181d-5p.

In one aspect, the invention provides a panel of isolated nucleic acid biomarkers comprising two or more of the nucleic acid biomarkers listed in Table 4. In another aspect, the invention provides a panel of isolated nucleic acid biomarkers comprising a combination of nucleic acid biomarker pairs as listed in Table 6 in combination with the two or more nucleic acid biomarkers listed in Table 4.

In some embodiments, a panel described herein comprises hsa-miR-140-3p/hsa-miR-516b-5p. In some embodiments, a panel described herein comprises hsa-miR-155-5p/hsa-miR-625-3p. In some embodiments, a panel described herein comprises hsa-miR-30c-5p/hsa-miR-27b-3p. In some embodiments, a panel described herein comprises hsa-miR-25-5p/hsa-miR-516b-5p. In some embodiments, a panel described herein comprises hsa-miR-484/hsa-miR-516b-5p. In some embodiments, a panel described herein comprises hsa-miR-516b-5p/hsa-let-7b-5p. In some embodiments, a panel described herein comprises hsa-miR-30c-5p/hsa-miR-7-5p. In some embodiments, a panel described herein comprises hsa-miR-516b-5p/hsa-miR-486-5p. In some embodiments, a panel described herein comprises hsa-miR-516b-5p/hsa-miR-150-5p. In some embodiments, a panel described herein comprises hsa-miR-485-5p/hsa-miR-516b-5p. In some embodiments, a panel described herein comprises hsa-miR-7-5p/hsa-miR-485-5p. In some embodiments, a panel described herein comprises hsa-miR-516b-5p/hsa-let-7g-5p. In some embodiments, a panel described herein comprises hsa-miR-30c-5p/hsa-miR-16-5p. In some embodiments, a panel described herein comprises hsa-miR-516b-5p/hsa-miR-423-5p. In some embodiments, a panel described herein comprises hsa-miR-30c-5p/hsa-miR-30a-5p. In some embodiments, a panel described herein comprises hsa-miR-516b-5p/hsa-miR-92a-3p. In some embodiments, a panel described herein comprises hsa-miR-186-5p/hsa-miR-516b-5p. In some embodiments, a panel described herein comprises hsa-miR-155-5p/hsa-miR-27b-3p. In some embodiments, a panel described herein comprises hsa-miR-30e-5p/hsa-miR-516b-5p. In some embodiments, a panel described herein comprises hsa-miR-516b-5p/hsa-let-7d-5p. In some embodiments, a panel described herein comprises hsa-miR-516b-5p/hsa-miR-103a-3p. In some embodiments, a panel described herein comprises hsa-miR-516b-5p/hsa-miR-126-3p. In some embodiments, a panel described herein comprises hsa-miR-99b-3p/hsa-miR-486-3p. In some embodiments, a panel described herein comprises hsa-miR-6741-5p/hsa-miR-760. In some embodiments, a panel described herein comprises hsa-miR-20a-5p/hsa-miR-485-5p. In some embodiments, a panel described herein comprises hsa-miR-23a-3p/hsa-miR-27b-3p. In some embodiments, a panel described herein comprises hsa-miR-516b-5p/hsa-miR-151a-3p. In some embodiments, a panel described herein comprises hsa-miR-30a-5p/hsa-miR-485-5p. In some embodiments, a panel described herein comprises hsa-miR-516b-5p/hsa-let-7a-5p. In some embodiments, a panel described herein comprises hsa-miR-30c-5p/hsa-miR-27a-3p. In some embodiments, a panel described herein comprises hsa-miR-485-5p/hsa-miR-7-5p. In some embodiments, a panel described herein comprises hsa-miR-516b-5p/hsa-miR-222-3p. In some embodiments, a panel described herein comprises hsa-miR-23a-3p/hsa-miR-30a-5p. In some embodiments, a panel described herein comprises hsa-miR-433-3p/hsa-miR-345-5p. In some embodiments, a panel described herein comprises hsa-miR-516b-5p/hsa-let-7f-5p. In some embodiments, a panel described herein comprises hsa-miR-485-5p/hsa-miR-183-5p. In some embodiments, a panel described herein comprises hsa-miR-516b-5p/hsa-miR-185-5p. In some embodiments, a panel described herein comprises hsa-let-7i-5p/hsa-miR-485-5p. In some embodiments, a panel described herein comprises hsa-miR-4433b-5p/hsa-miR-516b-5p. In some embodiments, a panel described herein comprises hsa-miR-516b-5p/hsa-miR-191-5p. In some embodiments, a panel described herein comprises hsa-miR-516b-5p/hsa-miR-3615. In some embodiments, a panel described herein comprises hsa-miR-100-5p/hsa-miR-516b-5p. In some embodiments, a panel described herein comprises hsa-miR-516b-5p/hsa-miR-10b-5p. In some embodiments, a panel described herein comprises hsa-miR-485-5p/hsa-miR-27a-3p. In some embodiments, a panel described herein comprises hsa-miR-99a-5p/hsa-miR-485-5p. In some embodiments, a panel described herein comprises hsa-miR-99b-3p/hsa-miR-6852-5p. In some embodiments, a panel described herein comprises hsa-miR-363-3p/hsa-miR-485-5p. In some embodiments, a panel described herein comprises hsa-miR-516b-5p/hsa-miR-484. In some embodiments, a panel described herein comprises hsa-miR-516b-5p/hsa-let-7i-5p. In some embodiments, a panel described herein comprises hsa-miR-23a-3p/hsa-miR-181a-5p. In some embodiments, a panel described herein comprises hsa-miR-7-5p/hsa-miR-516b-5p. In some embodiments, a panel described herein comprises hsa-miR-516b-5p/hsa-miR-328-3p. In some embodiments, a panel described herein comprises hsa-miR-181a-5p/hsa-miR-485-5p. In some embodiments, a panel described herein comprises hsa-miR-155-5p/hsa-miR-30a-5p. In some embodiments, a panel described herein comprises hsa-miR-155-5p/hsa-let-7i-5p. In some embodiments, a panel described herein comprises hsa-miR-98-5p/hsa-miR-485-5p. In some embodiments, a panel described herein comprises hsa-miR-485-5p/hsa-miR-127-3p. In some embodiments, a panel described herein comprises hsa-miR-320b/hsa-miR-485-5p. In some embodiments, a panel described herein comprises hsa-miR-516b-5p/hsa-miR-25-3p. In some embodiments, a panel described herein comprises hsa-miR-516b-5p/hsa-miR-148a-3p. In some embodiments, a panel described herein comprises hsa-miR-516b-5p/hsa-miR-128-3p. In some embodiments, a panel described herein comprises hsa-miR-516b-5p/hsa-miR-320a. In some embodiments, a panel described herein comprises hsa-miR-516b-5p/hsa-miR-744-5p. In some embodiments, a panel described herein comprises hsa-miR-30c-5p/hsa-miR-186-5p. In some embodiments, a panel described herein comprises hsa-miR-516b-5p/hsa-miR-629-5p. In some embodiments, a panel described herein comprises hsa-miR-23a-3p/hsa-miR-93-5p. In some embodiments, a panel described herein comprises hsa-miR-127-3p/hsa-miR-485-5p. In some embodiments, a panel described herein comprises hsa-miR-155-5p/hsa-miR-451a. In some embodiments, a panel described herein comprises hsa-miR-30c-5p/hsa-miR-26b-5p. In some embodiments, a panel described herein comprises hsa-miR-4732-3p/hsa-miR-516b-5p. In some embodiments, a panel described herein comprises hsa-miR-7-5p/hsa-miR-941. In some embodiments, a panel described herein comprises hsa-miR-451a/hsa-miR-485-5p. In some embodiments, a panel described herein comprises hsa-miR-374b-5p/hsa-miR-885-3p. In some embodiments, a panel described herein comprises hsa-miR-516b-5p/hsa-miR-1307-3p. In some embodiments, a panel described herein comprises hsa-miR-516b-5p/hsa-miR-30d-5p. In some embodiments, a panel described herein comprises hsa-miR-3605-5p/hsa-miR-425-3p. In some embodiments, a panel described herein comprises hsa-miR-501-3p/hsa-miR-516b-5p. In some embodiments, a panel described herein comprises hsa-miR-125b-5p/hsa-miR-485-5p. In some embodiments, a panel described herein comprises hsa-miR-26b-5p/hsa-miR-485-5p. In some embodiments, a panel described herein comprises hsa-miR-30c-5p/hsa-miR-99a-5p. In some embodiments, a panel described herein comprises hsa-miR-516b-5p/hsa-miR-127-3p. In some embodiments, a panel described herein comprises hsa-miR-501-3p/hsa-miR-485-5p. In some embodiments, a panel described herein comprises hsa-miR-30c-5p/hsa-miR-451a. In some embodiments, a panel described herein comprises hsa-miR-21-5p/hsa-miR-485-5p. In some embodiments, a panel described herein comprises hsa-miR-516b-5p/hsa-miR-423-3p. In some embodiments, a panel described herein comprises hsa-miR-516b-5p/hsa-miR-146b-5p. In some embodiments, a panel described herein comprises hsa-miR-330-5p/hsa-miR-92b-5p. In some embodiments, a panel described herein comprises hsa-miR-4443/hsa-miR-485-5p. In some embodiments, a panel described herein comprises hsa-miR-30c-5p/hsa-let-7i-5p. In some embodiments, a panel described herein comprises hsa-miR-516b-5p/hsa-miR-425-5p. In some embodiments, a panel described herein comprises hsa-miR-516b-5p/hsa-miR-24-3p. In some embodiments, a panel described herein comprises hsa-miR-146b-5p/hsa-miR-485-5p. In some embodiments, a panel described herein comprises hsa-miR-760/hsa-miR-378g. In some embodiments, a panel described herein comprises hsa-miR-3615/hsa-miR-485-5p. In some embodiments, a panel described herein comprises hsa-miR-140-3p/hsa-miR-485-5p. In some embodiments, a panel described herein comprises hsa-miR-30c-5p/hsa-miR-125a-5p. In some embodiments, a panel described herein comprises hsa-miR-378c/hsa-miR-148a-5p. In some embodiments, a panel described herein comprises hsa-let-7b-5p/hsa-miR-485-5p. In some embodiments, a panel described herein comprises hsa-miR-516b-5p/hsa-miR-146a-5p. In some embodiments, a panel described herein comprises hsa-miR-516b-5p/hsa-miR-451a.

In some embodiments, a panel described herein comprises hsa-miR-140-3p/hsa-miR-516b-5p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-155-5p/hsa-miR-625-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-30c-5p/hsa-miR-27b-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-140-3p/hsa-miR-516b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-155-5p/hsa-miR-625-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-30c-5p/hsa-miR-27b-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-516b-5p/hsa-let-7b-5p, hsa-miR-516b-5p/hsa-miR-423-5p and hsa-miR-155-5p/hsa-let-7i-5p.

In some embodiments, a panel described herein comprises hsa-miR-516b-5p/hsa-let-7b-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-516b-5p/hsa-miR-423-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-155-5p/hsa-let-7i-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a panel described herein comprises hsa-miR-516b-5p/hsa-let-7b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-516b-5p/hsa-miR-423-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a panel described herein comprises hsa-miR-155-5p/hsa-let-7i-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In one aspect, the invention provides a panel of isolated nucleic acid biomarkers comprising a pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, the biomarker panel comprises isolated nucleic acid biomarkers comprising hsa-miR-30c-5p/hsa-miR-27b-3p, hsa-miR-516b-5p/hsa-miR-150-5p, hsa-miR-30c-5p/hsa-miR-16-5p, hsa-miR-30c-5p/hsa-miR-30a-5p, hsa-miR-516b-5p/hsa-miR-92a-3p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-23a-3p/hsa-miR-27b-3p, hsa-miR-30a-5p/hsa-miR-485-5p, hsa-miR-516b-5p/hsa-let-7a-5p, hsa-miR-516b-5p/hsa-miR-222-3p, hsa-miR-516b-5p/hsa-let-7f-5p, hsa-miR-485-5p/hsa-miR-183-5p, hsa-miR-4433b-5p/hsa-miR-516b-5p, hsa-miR-516b-5p/hsa-miR-10b-5p, hsa-miR-516b-5p/hsa-miR-328-3p, hsa-miR-155-5p/hsa-miR-30a-5p, hsa-miR-516b-5p/hsa-miR-128-3p, hsa-miR-516b-5p/hsa-miR-744-5p, hsa-miR-516b-5p/hsa-miR-629-5p, hsa-miR-23a-3p/hsa-miR-93-5p, hsa-miR-3605-5p/hsa-miR-425-3p, hsa-miR-21-5p/hsa-miR-485-5p, hsa-miR-516b-5p/hsa-miR-24-3p, and/or hsa-miR-378c/hsa-miR-148a-5p.

In some embodiments, the biomarker panel comprises isolated nucleic acid biomarkers comprising hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, and/or hsa-miR-484/hsa-miR-516b-5p.

In some embodiments, the biomarker panel comprises isolated nucleic acid biomarkers comprising hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, and/or hsa-miR-30c-5p/hsa-miR-7-5p.

In some embodiments, the biomarker panel comprises isolated nucleic acid biomarkers comprising hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, and/or hsa-miR-485-5p/hsa-miR-516b-5p.

In a further aspect, the invention provides a panel of isolated nucleic acid biomarkers comprising at least one of the nucleic acid biomarkers listed in Table 5. In additional aspects, the invention provides a panel of isolated nucleic acid biomarkers comprising at least one pair of the nucleic acid biomarkers listed in Table 5.

In some embodiments, a panel described herein comprises nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p.

In some embodiments, a panel described herein comprises hsa-miR-30c-5p- In some embodiments, a panel described herein comprises hsa-miR-6842-3p. In some embodiments, a panel described herein comprises hsa-miR-485-5p. In some embodiments, a panel described herein comprises hsa-miR-331-3p. In some embodiments, a panel described herein comprises hsa-miR-517a-3p-hsa-miR-517b-3p. In some embodiments, a panel described herein comprises hsa-miR-516b-5p. In some embodiments, a panel described herein comprises hsa-miR-760. In some embodiments, a panel described herein comprises hsa-miR-6852-5p. In some embodiments, a panel described herein comprises hsa-miR-1323. In some embodiments, a panel described herein comprises hsa-miR-155-5p. In some embodiments, a panel described herein comprises hsa-miR-6747-3p. In some embodiments, a panel described herein comprises hsa-miR-181d-5p. In some embodiments, a panel described herein comprises hsa-miR-4446-3p.

In some embodiments, a panel described herein comprises hsa-miR-485-5p and at least one biomarker selected from the group consisting of hsa-miR-7-5p, hsa-miR-501-3p, hsa-miR-363-3p, hsa-miR-182-5p, hsa-miR-127-3p, hsa-miR-140-3p, hsa-miR-26b-5p.

In some embodiments, a panel described herein comprises hsa-miR-485-5p and at least one pair biomarker selected from the group consisting of hsa-miR-27b-3p, hsa-let-7i-5p, hsa-miR-99a-5p, hsa-miR-409-3p.

In some embodiments, a panel described herein comprises hsa-miR-485-5p and two or more biomarkers selected from the group consisting of the additional miRNAs in Table 5.

In one aspect, the invention provides a composition of labeled and/or amplified nucleic acid molecules, for example, amplified labeled nucleic acid molecules, that correspond to two or more of the nucleic acid biomarkers listed in Tables 3, 4 or 5. In another aspect, the invention provides a composition of labeled and/or amplified nucleic acid molecules, for example, amplified labeled nucleic acid molecules, that correspond to any one or more of the panels of isolated nucleic acid biomarkers of Table 6. In another aspect, the invention provides a composition of labeled and/or amplified nucleic acid molecules, for example, amplified labeled nucleic acid molecules, that correspond to at least one of the nucleic acid biomarkers listed in Tables 3, 4 or 5, in addition to labeled and/or amplified nucleic acid molecules, for example, amplified labeled nucleic acid molecules, that correspond to any one or more of the panels of isolated nucleic acid biomarkers of Table 6.

In one aspect, the invention provides a composition of labeled and/or amplified nucleic acid molecules, for example, amplified labeled nucleic acid molecules, that correspond to two or more of the nucleic acid biomarkers listed in Table 3. In a further aspect, the invention provides a composition of labeled and/or amplified nucleic acid molecules, for example, amplified labeled nucleic acid molecules, that correspond to at least one of the nucleic acid biomarkers listed in Table 3 in addition to labeled and/or amplified nucleic acid molecules, for example, amplified labeled nucleic acid molecules, that correspond one or more of the panels of isolated nucleic acid biomarkers of Table 6

In some embodiments, a composition described herein comprises hsa-miR-516b-5p. In some embodiments, a composition described herein comprises hsa-miR-30c-5p. In some embodiments, a composition described herein comprises hsa-miR-1-3p. In some embodiments, a composition described herein comprises hsa-miR-331-3p. In some embodiments, a composition described herein comprises hsa-miR-485-5p. In some embodiments, a composition described herein comprises hsa-miR-181d-5p. In some embodiments, a composition described herein comprises hsa-miR-23a-3p. In some embodiments, a composition described herein comprises hsa-miR-378d. In some embodiments, a composition described herein comprises hsa-miR-155-5p. In some embodiments, a composition described herein comprises hsa-miR-99b-3p. In some embodiments, a composition described herein comprises hsa-miR-1249-3p. In some embodiments, a composition described herein comprises hsa-miR-142-3p. In some embodiments, a composition described herein comprises hsa-miR-4433b-5p. In some embodiments, a composition described herein comprises hsa-miR-1273h-3p. In some embodiments, a composition described herein comprises hsa-miR-18a-3p. In some embodiments, a composition described herein comprises hsa-miR-4746-5p. In some embodiments, a composition described herein comprises hsa-miR-27a-5p. In some embodiments, a composition described herein comprises hsa-miR-654-3p. In some embodiments, a composition described herein comprises hsa-miR-7-5p. In some embodiments, a composition described herein comprises hsa-miR-941.

In some embodiments, a composition described herein comprises hsa-miR-516b-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-30c-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-1-3p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-516b-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-30c-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-1-3p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In one aspect, the invention provides a composition of labeled and/or amplified nucleic acid molecules, for example, amplified labeled nucleic acid molecules, that correspond to two or more nucleic acid biomarkers selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, the invention provides a composition of labeled and/or amplified nucleic acid molecules, for example, amplified labeled nucleic acid molecules, that correspond to nucleic acid biomarkers hsa-miR-1-3p, hsa-miR-181d-5p, hsa-miR-378d, hsa-miR-4433b-5p, and/or hsa-miR-27a-5p. In some embodiments, the invention provides a composition of labeled and/or amplified nucleic acid molecules, for example, amplified labeled nucleic acid molecules, that correspond to nucleic acid biomarkers hsa-miR-516b-5p and/or hsa-miR-30c-5p. In some embodiments, the invention provides a composition of labeled and/or amplified nucleic acid molecules, for example, amplified labeled nucleic acid molecules, that correspond to nucleic acid biomarkers hsa-miR-516b-5p, hsa-miR-30c-5p, hsa-miR-1-3p, and/or hsa-miR-331-3p. In some embodiments, the invention provides a composition of labeled and/or amplified nucleic acid molecules, for example, amplified labeled nucleic acid molecules, that correspond to nucleic acid biomarkers hsa-miR-516b-5p, hsa-miR-30c-5p, hsa-miR-1-3p, hsa-miR-331-3p, hsa-miR-485-5p, and/or hsa-miR-181d-5p.

In one aspect, the invention provides a composition of labeled and/or amplified nucleic acid molecules that correspond to a pair of biomarkers selected from the group consisting of the biomarker pairs listed in Table 4.

In some embodiments, a composition described herein comprises hsa-miR-140-3p/hsa-miR-516b-5p. In some embodiments, a composition described herein comprises hsa-miR-155-5p/hsa-miR-625-3p. In some embodiments, a composition described herein comprises hsa-miR-30c-5p/hsa-miR-27b-3p. In some embodiments, a composition described herein comprises hsa-miR-25-5p/hsa-miR-516b-5p. In some embodiments, a composition described herein comprises hsa-miR-484/hsa-miR-516b-5p. In some embodiments, a composition described herein comprises hsa-miR-516b-5p/hsa-let-7b-5p. In some embodiments, a composition described herein comprises hsa-miR-30c-5p/hsa-miR-7-5p. In some embodiments, a composition described herein comprises hsa-miR-516b-5p/hsa-miR-486-5p. In some embodiments, a composition described herein comprises hsa-miR-516b-5p/hsa-miR-150-5p. In some embodiments, a composition described herein comprises hsa-miR-485-5p/hsa-miR-516b-5p. In some embodiments, a composition described herein comprises hsa-miR-7-5p/hsa-miR-485-5p. In some embodiments, a composition described herein comprises hsa-miR-516b-5p/hsa-let-7g-5p. In some embodiments, a composition described herein comprises hsa-miR-30c-5p/hsa-miR-16-5p. In some embodiments, a composition described herein comprises hsa-miR-516b-5p/hsa-miR-423-5p. In some embodiments, a composition described herein comprises hsa-miR-30c-5p/hsa-miR-30a-5p. In some embodiments, a composition described herein comprises hsa-miR-516b-5p/hsa-miR-92a-3p. In some embodiments, a composition described herein comprises hsa-miR-186-5p/hsa-miR-516b-5p. In some embodiments, a composition described herein comprises hsa-miR-155-5p/hsa-miR-27b-3p. In some embodiments, a composition described herein comprises hsa-miR-30e-5p/hsa-miR-516b-5p. In some embodiments, a composition described herein comprises hsa-miR-516b-5p/hsa-let-7d-5p. In some embodiments, a composition described herein comprises hsa-miR-516b-5p/hsa-miR-103a-3p. In some embodiments, a composition described herein comprises hsa-miR-516b-5p/hsa-miR-126-3p. In some embodiments, a composition described herein comprises hsa-miR-99b-3p/hsa-miR-486-3p. In some embodiments, a composition described herein comprises hsa-miR-6741-5p/hsa-miR-760. In some embodiments, a composition described herein comprises hsa-miR-20a-5p/hsa-miR-485-5p. In some embodiments, a composition described herein comprises hsa-miR-23a-3p/hsa-miR-27b-3p. In some embodiments, a composition described herein comprises hsa-miR-516b-5p/hsa-miR-151a-3p. In some embodiments, a composition described herein comprises hsa-miR-30a-5p/hsa-miR-485-5p. In some embodiments, a composition described herein comprises hsa-miR-516b-5p/hsa-let-7a-5p. In some embodiments, a composition described herein comprises hsa-miR-30c-5p/hsa-miR-27a-3p. In some embodiments, a composition described herein comprises hsa-miR-485-5p/hsa-miR-7-5p. In some embodiments, a composition described herein comprises hsa-miR-516b-5p/hsa-miR-222-3p. In some embodiments, a composition described herein comprises hsa-miR-23a-3p/hsa-miR-30a-5p. In some embodiments, a composition described herein comprises hsa-miR-433-3p/hsa-miR-345-5p. In some embodiments, a composition described herein comprises hsa-miR-516b-5p/hsa-let-7f-5p. In some embodiments, a composition described herein comprises hsa-miR-485-5p/hsa-miR-183-5p. In some embodiments, a composition described herein comprises hsa-miR-516b-5p/hsa-miR-185-5p. In some embodiments, a composition described herein comprises hsa-let-7i-5p/hsa-miR-485-5p. In some embodiments, a composition described herein comprises hsa-miR-4433b-5p/hsa-miR-516b-5p. In some embodiments, a composition described herein comprises hsa-miR-516b-5p/hsa-miR-191-5p. In some embodiments, a composition described herein comprises hsa-miR-516b-5p/hsa-miR-3615. In some embodiments, a composition described herein comprises hsa-miR-100-5p/hsa-miR-516b-5p. In some embodiments, a composition described herein comprises hsa-miR-516b-5p/hsa-miR-10b-5p. In some embodiments, a composition described herein comprises hsa-miR-485-5p/hsa-miR-27a-3p. In some embodiments, a composition described herein comprises hsa-miR-99a-5p/hsa-miR-485-5p. In some embodiments, a composition described herein comprises hsa-miR-99b-3p/hsa-miR-6852-5p. In some embodiments, a composition described herein comprises hsa-miR-363-3p/hsa-miR-485-5p. In some embodiments, a composition described herein comprises hsa-miR-516b-5p/hsa-miR-484. In some embodiments, a composition described herein comprises hsa-miR-516b-5p/hsa-let-7i-5p. In some embodiments, a composition described herein comprises hsa-miR-23a-3p/hsa-miR-181a-5p. In some embodiments, a composition described herein comprises hsa-miR-7-5p/hsa-miR-516b-5p. In some embodiments, a composition described herein comprises hsa-miR-516b-5p/hsa-miR-328-3p. In some embodiments, a composition described herein comprises hsa-miR-181a-5p/hsa-miR-485-5p. In some embodiments, a composition described herein comprises hsa-miR-155-5p/hsa-miR-30a-5p. In some embodiments, a composition described herein comprises hsa-miR-155-5p/hsa-let-7i-5p. In some embodiments, a composition described herein comprises hsa-miR-98-5p/hsa-miR-485-5p. In some embodiments, a composition described herein comprises hsa-miR-485-5p/hsa-miR-127-3p. In some embodiments, a composition described herein comprises hsa-miR-320b/hsa-miR-485-5p. In some embodiments, a composition described herein comprises hsa-miR-516b-5p/hsa-miR-25-3p. In some embodiments, a composition described herein comprises hsa-miR-516b-5p/hsa-miR-148a-3p. In some embodiments, a composition described herein comprises hsa-miR-516b-5p/hsa-miR-128-3p. In some embodiments, a composition described herein comprises hsa-miR-516b-5p/hsa-miR-320a. In some embodiments, a composition described herein comprises hsa-miR-516b-5p/hsa-miR-744-5p. In some embodiments, a composition described herein comprises hsa-miR-30c-5p/hsa-miR-186-5p. In some embodiments, a composition described herein comprises hsa-miR-516b-5p/hsa-miR-629-5p. In some embodiments, a composition described herein comprises hsa-miR-23a-3p/hsa-miR-93-5p. In some embodiments, a composition described herein comprises hsa-miR-127-3p/hsa-miR-485-5p. In some embodiments, a composition described herein comprises hsa-miR-155-5p/hsa-miR-451a. In some embodiments, a composition described herein comprises hsa-miR-30c-5p/hsa-miR-26b-5p. In some embodiments, a composition described herein comprises hsa-miR-4732-3p/hsa-miR-516b-5p. In some embodiments, a composition described herein comprises hsa-miR-7-5p/hsa-miR-941. In some embodiments, a composition described herein comprises hsa-miR-451a/hsa-miR-485-5p. In some embodiments, a composition described herein comprises hsa-miR-374b-5p/hsa-miR-885-3p. In some embodiments, a composition described herein comprises hsa-miR-516b-5p/hsa-miR-1307-3p. In some embodiments, a composition described herein comprises hsa-miR-516b-5p/hsa-miR-30d-5p. In some embodiments, a composition described herein comprises hsa-miR-3605-5p/hsa-miR-425-3p. In some embodiments, a composition described herein comprises hsa-miR-501-3p/hsa-miR-516b-5p. In some embodiments, a composition described herein comprises hsa-miR-125b-5p/hsa-miR-485-5p. In some embodiments, a composition described herein comprises hsa-miR-26b-5p/hsa-miR-485-5p. In some embodiments, a composition described herein comprises hsa-miR-30c-5p/hsa-miR-99a-5p. In some embodiments, a composition described herein comprises hsa-miR-516b-5p/hsa-miR-127-3p. In some embodiments, a composition described herein comprises hsa-miR-501-3p/hsa-miR-485-5p. In some embodiments, a composition described herein comprises hsa-miR-30c-5p/hsa-miR-451a. In some embodiments, a composition described herein comprises hsa-miR-21-5p/hsa-miR-485-5p. In some embodiments, a composition described herein comprises hsa-miR-516b-5p/hsa-miR-423-3p. In some embodiments, a composition described herein comprises hsa-miR-516b-5p/hsa-miR-146b-5p. In some embodiments, a composition described herein comprises hsa-miR-330-5p/hsa-miR-92b-5p. In some embodiments, a composition described herein comprises hsa-miR-4443/hsa-miR-485-5p. In some embodiments, a composition described herein comprises hsa-miR-30c-5p/hsa-let-7i-5p. In some embodiments, a composition described herein comprises hsa-miR-516b-5p/hsa-miR-425-5p. In some embodiments, a composition described herein comprises hsa-miR-516b-5p/hsa-miR-24-3p. In some embodiments, a composition described herein comprises hsa-miR-146b-5p/hsa-miR-485-5p. In some embodiments, a composition described herein comprises hsa-miR-760/hsa-miR-378g. In some embodiments, a composition described herein comprises hsa-miR-3615/hsa-miR-485-5p. In some embodiments, a composition described herein comprises hsa-miR-140-3p/hsa-miR-485-5p. In some embodiments, a composition described herein comprises hsa-miR-30c-5p/hsa-miR-125a-5p. In some embodiments, a composition described herein comprises hsa-miR-378c/hsa-miR-148a-5p. In some embodiments, a composition described herein comprises hsa-let-7b-5p/hsa-miR-485-5p. In some embodiments, a composition described herein comprises hsa-miR-516b-5p/hsa-miR-146a-5p. In some embodiments, a composition described herein comprises hsa-miR-516b-5p/hsa-miR-451a.

In some embodiments, a composition described herein comprises hsa-miR-140-3p/hsa-miR-516b-5p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-155-5p/hsa-miR-625-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-30c-5p/hsa-miR-27b-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-140-3p/hsa-miR-516b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-155-5p/hsa-miR-625-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-30c-5p/hsa-miR-27b-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-516b-5p/hsa-let-7b-5p, hsa-miR-516b-5p/hsa-miR-423-5p and hsa-miR-155-5p/hsa-let-7i-5p.

In some embodiments, a composition described herein comprises hsa-miR-516b-5p/hsa-let-7b-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-516b-5p/hsa-miR-423-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-155-5p/hsa-let-7i-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a composition described herein comprises hsa-miR-516b-5p/hsa-let-7b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-516b-5p/hsa-miR-423-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a composition described herein comprises hsa-miR-155-5p/hsa-let-7i-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In one aspect, the invention provides a composition of labeled and/or amplified nucleic acid molecules, for example, amplified labeled nucleic acid molecules, that correspond to a pair of biomarker selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments of the pair of biomarkers or panel of biomarkers comprising a pair of biomarkers, the pair of biomarkers is selected from the group consisting of hsa-miR-30c-5p/hsa-miR-27b-3p, hsa-miR-516b-5p/hsa-miR-150-5p, hsa-miR-30c-5p/hsa-miR-16-5p, hsa-miR-30c-5p/hsa-miR-30a-5p, hsa-miR-516b-5p/hsa-miR-92a-3p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-23a-3p/hsa-miR-27b-3p, hsa-miR-30a-5p/hsa-miR-485-5p, hsa-miR-516b-5p/hsa-let-7a-5p, hsa-miR-516b-5p/hsa-miR-222-3p, hsa-miR-516b-5p/hsa-let-7f-5p, hsa-miR-485-5p/hsa-miR-183-5p, hsa-miR-4433b-5p/hsa-miR-516b-5p, hsa-miR-516b-5p/hsa-miR-10b-5p, hsa-miR-516b-5p/hsa-miR-328-3p, hsa-miR-155-5p/hsa-miR-30a-5p, hsa-miR-516b-5p/hsa-miR-128-3p, hsa-miR-516b-5p/hsa-miR-744-5p, hsa-miR-516b-5p/hsa-miR-629-5p, hsa-miR-23a-3p/hsa-miR-93-5p, hsa-miR-3605-5p/hsa-miR-425-3p, hsa-miR-21-5p/hsa-miR-485-5p, hsa-miR-516b-5p/hsa-miR-24-3p, and hsa-miR-378c/hsa-miR-148a-5p.

In some embodiments, the invention provides a composition comprising hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, the invention provides a composition comprising hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, the invention provides a composition comprising hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, the invention provides a composition comprising hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, the invention provides a composition comprising hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, the invention provides a composition comprising hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, the invention provides a composition comprising hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, the invention provides a composition comprising hsa-miR-7-5p/hsa-miR-485-5p; hsa-miR-1306-3p/hsa-miR-664a-5p; and hsa-miR-501-3p/hsa-miR-484.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, the invention provides a composition comprising hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, the invention provides a composition comprising hsa-miR-7-5p/hsa-miR-485-5p; hsa-miR-1306-3p/hsa-miR-664a-5p; and hsa-miR-628-3p/hsa-miR-19b-3p.

In some embodiments, the invention provides a composition comprising hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, the invention provides a composition comprising hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, the invention provides a composition comprising hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, the invention provides a composition comprising hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p.

In some embodiments, the invention provides a composition comprising hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, the invention provides a composition comprising hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, the invention provides a composition comprising hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, the invention provides a composition comprising hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, the invention provides a composition comprising hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, the invention provides a composition comprising hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, the invention provides a composition comprising hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, the invention provides a composition comprising hsa-miR-7-5p/hsa-miR-485-5p; hsa-miR-30c-5p/hsa-miR-125a-5p; and hsa-miR-1-3p/hsa-miR-345-5p.

In some embodiments, the invention provides a composition comprising hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, the invention provides a composition comprising hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, the invention provides a composition comprising hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p.

In some embodiments, the invention provides a composition comprising hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, the invention provides a composition comprising hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, the invention provides a composition comprising hsa-miR-7-5p/hsa-miR-485-5p; hsa-miR-1260b/hsa-mir-320c-2; and hsa-miR-433-3p/hsa-miR-345-5p.

In some embodiments, the invention provides a composition comprising hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, the invention provides a composition comprising hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, the invention provides a composition comprising hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, the invention provides a composition comprising hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, the invention provides a composition comprising hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, the invention provides a composition comprising hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, the invention provides a composition comprising hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, the invention provides a composition comprising hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, the invention provides a composition comprising hsa-miR-7-5p/hsa-miR-485-5p; hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, the invention provides a composition comprising hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, the invention provides a composition comprising hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, the invention provides a composition comprising hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, the invention provides a composition comprising hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, the invention provides a composition comprising hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, the invention provides a composition comprising hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, the invention provides a composition comprising hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, the invention provides a composition comprising hsa-miR-7-5p/hsa-miR-485-5p; hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, the invention provides a composition comprising hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a composition comprising hsa-miR-7-5p/hsa-miR-485-5p; hsa-miR-221-5p/hsa-miR-760; hsa-miR-99b-3p/hsa-miR-340-5p; and hsa-miR-1306-3p/hsa-miR-150-3p.

In some embodiments, the invention provides a composition comprising hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, the invention provides a composition comprising hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, the invention provides a composition comprising hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, the invention provides a composition comprising hsa-miR-330-5p/hsa-miR-223-3p; hsa-miR-7-5p/hsa-miR-485-5p; and hsa-miR-511-5p/hsa-miR-148a-5p.

In some embodiments, the invention provides a composition comprising hsa-miR-7-5p/hsa-miR-485-5p; hsa-miR-1306-3p/hsa-miR-664a-5p; hsa-miR-30a-5p/hsa-miR-941; and hsa-miR-30c-5p/hsa-miR-27b-3p.

In some embodiments of the pair of biomarkers or panel of biomarkers comprising a pair of biomarkers, the pair of biomarkers is selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, and hsa-miR-484/hsa-miR-516b-5p.

In some embodiments of the pair of biomarkers or panel of biomarkers comprising a pair of biomarkers, the pair of biomarkers is selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, and hsa-miR-30c-5p/hsa-miR-7-5p.

In some embodiments of the pair of biomarkers or panel of biomarkers comprising a pair of biomarkers, the pair of biomarkers is selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, and hsa-miR-485-5p/hsa-miR-516b-5p.

In one aspect, the invention provides a composition of labeled and/or amplified nucleic acid molecules that correspond to at least one of the nucleic acid biomarkers listed in Table 5. In an additional aspect, the invention provides a composition of labeled and/or amplified nucleic acid molecules that correspond to at least one pair of the nucleic acid biomarkers listed in Table 5

In some embodiments, a composition described herein comprises nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p

In some embodiments, a composition described herein comprises hsa-miR-30c-5p. In some embodiments, a composition described herein comprises hsa-miR-6842-3p.

In some embodiments, a composition described herein comprises hsa-miR-485-5p. In some embodiments, a composition described herein comprises hsa-miR-331-3p. In some embodiments, a composition described herein comprises hsa-miR-517a-3p-hsa-miR-517b-3p. In some embodiments, a composition described herein comprises hsa-miR-516b-5p. In some embodiments, a composition described herein comprises hsa-miR-760. In some embodiments, a composition described herein comprises hsa-miR-6852-5p. In some embodiments, a composition described herein comprises hsa-miR-1323. In some embodiments, a composition described herein comprises hsa-miR-155-5p. In some embodiments, a composition described herein comprises hsa-miR-6747-3p. In some embodiments, a composition described herein comprises hsa-miR-181d-5p. In some embodiments, a composition described herein comprises hsa-miR-4446-3p.

In some embodiments, a composition described herein comprises hsa-miR-485-5p and at least one biomarker selected from the group consisting of hsa-miR-7-5p, hsa-miR-501-3p, hsa-miR-363-3p, hsa-miR-182-5p, hsa-miR-127-3p, hsa-miR-140-3p, hsa-miR-26b-5p.

In some embodiments, a composition described herein comprises hsa-miR-485-5p and at least one pair biomarker selected from the group consisting of hsa-miR-27b-3p, hsa-let-7i-5p, hsa-miR-99a-5p, hsa-miR-409-3p.

In some embodiments, a composition described herein comprises hsa-miR-485-5p and two or more biomarkers selected from Table 5.

In one aspect, the invention provides a method for determining a pregnant female's risk of developing placental dysfunction later in the pregnancy comprising measuring the amount of two or more of the nucleic acid biomarkers listed in Tables 3, 4 or 5, or a combination of nucleic acid biomarkers corresponding to a panel of nucleic acid biomarkers listed in Table 6, in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In one aspect, the invention provides a method for determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers listed in Tables 3, 4 or 5, or a combination of nucleic acid biomarkers corresponding to one or more of the panels of nucleic acid biomarkers listed in Table 6, in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In another aspect, this disclosure provides a method for determining a pregnant female's risk of developing placental dysfunction during pregnancy comprising measuring amounts of the combination of nucleic acid biomarker pairs from the panel of isolated nucleic acid biomarkers of any one or more of the panels of nucleic acid biomarkers from Table 6 in a biological sample obtained from the pregnant female, calculating a risk score based upon the measured amounts to determine the pregnant female's risk of developing placental dysfunction, and providing a risk score corresponding to the pregnant female's risk of developing placental dysfunction.

In another aspect, this disclosure can provide a method for determining a pregnant female's risk of developing placental dysfunction during pregnancy comprising producing labeled and/or amplified nucleic acid molecules that correspond to the composition of labeled and/or amplified nucleic acid molecules of the one or more panels of Table 6 in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

In some embodiments, a method described herein comprises producing labeled and/or amplified nucleic acid molecules that correspond to hsa-miR-516b-5p. In some embodiments, a method described herein comprises hsa-miR-30c-5p. In some embodiments, a method described herein comprises hsa-miR-1-3p. In some embodiments, a method described herein comprises hsa-miR-331-3p. In some embodiments, a method described herein comprises hsa-miR-485-5p. In some embodiments, a method described herein comprises hsa-miR-181d-5p. In some embodiments, a method described herein comprises hsa-miR-23a-3p. In some embodiments, a method described herein comprises hsa-miR-378d. In some embodiments, a method described herein comprises hsa-miR-155-5p. In some embodiments, a method described herein comprises hsa-miR-99b-3p. In some embodiments, a method described herein comprises hsa-miR-1249-3p. In some embodiments, a method described herein comprises hsa-miR-142-3p. In some embodiments, a method described herein comprises hsa-miR-4433b-5p. In some embodiments, a method described herein comprises hsa-miR-1273h-3p. In some embodiments, a method described herein comprises hsa-miR-18a-3p. In some embodiments, a method described herein comprises hsa-miR-4746-5p. In some embodiments, a method described herein comprises hsa-miR-27a-5p. In some embodiments, a method described herein comprises hsa-miR-654-3p. In some embodiments, a method described herein comprises hsa-miR-7-5p. In some embodiments, a method described herein comprises hsa-miR-941.

In some embodiments, a method described herein comprises producing labeled and/or amplified nucleic acid molecules that correspond to hsa-miR-516b-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-30c-5p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-1-3p and at least one biomarker selected from the group consisting of hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-516b-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-30c-5p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-1-3p and at least one pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-140-3p/hsa-miR-516b-5p. In some embodiments, a method described herein comprises producing labeled and/or amplified nucleic acid molecules that correspond to hsa-miR-155-5p/hsa-miR-625-3p. In some embodiments, a method described herein comprises producing labeled and/or amplified nucleic acid molecules that correspond to hsa-miR-30c-5p/hsa-miR-27b-3p. In some embodiments, a method described herein comprises producing labeled and/or amplified nucleic acid molecules that correspond to hsa-miR-25-5p/hsa-miR-516b-5p. In some embodiments, a method described herein comprises producing labeled and/or amplified nucleic acid molecules that correspond to hsa-miR-484/hsa-miR-516b-5p. In some embodiments, a method described herein comprises producing labeled and/or amplified nucleic acid molecules that correspond to hsa-miR-516b-5p/hsa-let-7b-5p. In some embodiments, a method described herein comprises producing labeled and/or amplified nucleic acid molecules that correspond to hsa-miR-30c-5p/hsa-miR-7-5p. In some embodiments, a method described herein comprises producing labeled and/or amplified nucleic acid molecules that correspond to hsa-miR-516b-5p/hsa-miR-486-5p. In some embodiments, a method described herein comprises producing labeled and/or amplified nucleic acid molecules that correspond to hsa-miR-516b-5p/hsa-miR-150-5p. In some embodiments, a method described herein comprises producing labeled and/or amplified nucleic acid molecules that correspond to hsa-miR-485-5p/hsa-miR-516b-5p. In some embodiments, a method described herein comprises producing labeled and/or amplified nucleic acid molecules that correspond to hsa-miR-7-5p/hsa-miR-485-5p. In some embodiments, a method described herein comprises producing labeled and/or amplified nucleic acid molecules that correspond to hsa-miR-516b-5p/hsa-let-7g-5p. In some embodiments, a method described herein comprises producing labeled and/or amplified nucleic acid molecules that correspond to hsa-miR-30c-5p/hsa-miR-16-5p. In some embodiments, a method described herein comprises producing labeled and/or amplified nucleic acid molecules that correspond to hsa-miR-516b-5p/hsa-miR-423-5p. In some embodiments, a method described herein comprises producing labeled and/or amplified nucleic acid molecules that correspond to hsa-miR-30c-5p/hsa-miR-30a-5p. In some embodiments, a method described herein comprises producing labeled and/or amplified nucleic acid molecules that correspond to hsa-miR-516b-5p/hsa-miR-92a-3p. In some embodiments, a method described herein comprises producing labeled and/or amplified nucleic acid molecules that correspond to hsa-miR-186-5p/hsa-miR-516b-5p. In some embodiments, a method described herein comprises producing labeled and/or amplified nucleic acid molecules that correspond to hsa-miR-155-5p/hsa-miR-27b-3p. In some embodiments, a method described herein comprises producing labeled and/or amplified nucleic acid molecules that correspond to hsa-miR-30e-5p/hsa-miR-516b-5p. In some embodiments, a method described herein comprises producing labeled and/or amplified nucleic acid molecules that correspond to hsa-miR-516b-5p/hsa-let-7d-5p. In some embodiments, a method described herein comprises producing labeled and/or amplified nucleic acid molecules that correspond to hsa-miR-516b-5p/hsa-miR-103a-3p. In some embodiments, a method described herein comprises producing labeled and/or amplified nucleic acid molecules that correspond to hsa-miR-516b-5p/hsa-miR-126-3p. In some embodiments, a method described herein comprises producing labeled and/or amplified nucleic acid molecules that correspond to hsa-miR-99b-3p/hsa-miR-486-3p. In some embodiments, a method described herein comprises producing labeled and/or amplified nucleic acid molecules that correspond to hsa-miR-6741-5p/hsa-miR-760. In some embodiments, a method described herein comprises producing labeled and/or amplified nucleic acid molecules that correspond to hsa-miR-20a-5p/hsa-miR-485-5p. In some embodiments, a method described herein comprises producing labeled and/or amplified nucleic acid molecules that correspond to hsa-miR-23a-3p/hsa-miR-27b-3p. In some embodiments, a method described herein comprises producing labeled and/or amplified nucleic acid molecules that correspond to hsa-miR-516b-5p/hsa-miR-151a-3p. In some embodiments, a method described herein comprises producing labeled and/or amplified nucleic acid molecules that correspond to hsa-miR-30a-5p/hsa-miR-485-5p. In some embodiments, a method described herein comprises producing labeled and/or amplified nucleic acid molecules that correspond to hsa-miR-516b-5p/hsa-let-7a-5p. In some embodiments, a method described herein comprises producing labeled and/or amplified nucleic acid molecules that correspond to hsa-miR-30c-5p/hsa-miR-27a-3p. In some embodiments, a method described herein comprises producing labeled and/or amplified nucleic acid molecules that correspond to hsa-miR-485-5p/hsa-miR-7-5p. In some embodiments, a method described herein comprises producing labeled and/or amplified nucleic acid molecules that correspond to hsa-miR-516b-5p/hsa-miR-222-3p. In some embodiments, a method described herein comprises producing labeled and/or amplified nucleic acid molecules that correspond to hsa-miR-23a-3p/hsa-miR-30a-5p. In some embodiments, a method described herein comprises producing labeled and/or amplified nucleic acid molecules that correspond to hsa-miR-433-3p/hsa-miR-345-5p. In some embodiments, a method described herein comprises producing labeled and/or amplified nucleic acid molecules that correspond to hsa-miR-516b-5p/hsa-let-7f-5p. In some embodiments, a method described herein comprises producing labeled and/or amplified nucleic acid molecules that correspond to hsa-miR-485-5p/hsa-miR-183-5p. In some embodiments, a method described herein comprises producing labeled and/or amplified nucleic acid molecules that correspond to hsa-miR-516b-5p/hsa-miR-185-5p. In some embodiments, a method described herein comprises producing labeled and/or amplified nucleic acid molecules that correspond to hsa-let-7i-5p/hsa-miR-485-5p. In some embodiments, a method described herein comprises producing labeled and/or amplified nucleic acid molecules that correspond to hsa-miR-4433b-5p/hsa-miR-516b-5p. In some embodiments, a method described herein comprises producing labeled and/or amplified nucleic acid molecules that correspond to hsa-miR-516b-5p/hsa-miR-191-5p. In some embodiments, a method described herein comprises producing labeled and/or amplified nucleic acid molecules that correspond to hsa-miR-516b-5p/hsa-miR-3615. In some embodiments, a method described herein comprises producing labeled and/or amplified nucleic acid molecules that correspond to hsa-miR-100-5p/hsa-miR-516b-5p. In some embodiments, a method described herein comprises producing labeled and/or amplified nucleic acid molecules that correspond to hsa-miR-516b-5p/hsa-miR-10b-5p. In some embodiments, a method described herein comprises producing labeled and/or amplified nucleic acid molecules that correspond to hsa-miR-485-5p/hsa-miR-27a-3p. In some embodiments, a method described herein comprises producing labeled and/or amplified nucleic acid molecules that correspond to hsa-miR-99a-5p/hsa-miR-485-5p. In some embodiments, a method described herein comprises producing labeled and/or amplified nucleic acid molecules that correspond to hsa-miR-99b-3p/hsa-miR-6852-5p. In some embodiments, a method described herein comprises producing labeled and/or amplified nucleic acid molecules that correspond to hsa-miR-363-3p/hsa-miR-485-5p. In some embodiments, a method described herein comprises producing labeled and/or amplified nucleic acid molecules that correspond to hsa-miR-516b-5p/hsa-miR-484. In some embodiments, a method described herein comprises producing labeled and/or amplified nucleic acid molecules that correspond to hsa-miR-516b-5p/hsa-let-7i-5p. In some embodiments, a method described herein comprises producing labeled and/or amplified nucleic acid molecules that correspond to hsa-miR-23a-3p/hsa-miR-181a-5p. In some embodiments, a method described herein comprises producing labeled and/or amplified nucleic acid molecules that correspond to hsa-miR-7-5p/hsa-miR-516b-5p. In some embodiments, a method described herein comprises producing labeled and/or amplified nucleic acid molecules that correspond to hsa-miR-516b-5p/hsa-miR-328-3p. In some embodiments, a method described herein comprises producing labeled and/or amplified nucleic acid molecules that correspond to hsa-miR-181a-5p/hsa-miR-485-5p. In some embodiments, a method described herein comprises producing labeled and/or amplified nucleic acid molecules that correspond to hsa-miR-155-5p/hsa-miR-30a-5p. In some embodiments, a method described herein comprises producing labeled and/or amplified nucleic acid molecules that correspond to hsa-miR-155-5p/hsa-let-7i-5p. In some embodiments, a method described herein comprises producing labeled and/or amplified nucleic acid molecules that correspond to hsa-miR-98-5p/hsa-miR-485-5p. In some embodiments, a method described herein comprises producing labeled and/or amplified nucleic acid molecules that correspond to hsa-miR-485-5p/hsa-miR-127-3p. In some embodiments, a method described herein comprises producing labeled and/or amplified nucleic acid molecules that correspond to hsa-miR-320b/hsa-miR-485-5p. In some embodiments, a method described herein comprises producing labeled and/or amplified nucleic acid molecules that correspond to hsa-miR-516b-5p/hsa-miR-25-3p. In some embodiments, a method described herein comprises producing labeled and/or amplified nucleic acid molecules that correspond to hsa-miR-516b-5p/hsa-miR-148a-3p. In some embodiments, a method described herein comprises producing labeled and/or amplified nucleic acid molecules that correspond to hsa-miR-516b-5p/hsa-miR-128-3p. In some embodiments, a method described herein comprises producing labeled and/or amplified nucleic acid molecules that correspond to hsa-miR-516b-5p/hsa-miR-320a. In some embodiments, a method described herein comprises producing labeled and/or amplified nucleic acid molecules that correspond to hsa-miR-516b-5p/hsa-miR-744-5p. In some embodiments, a method described herein comprises producing labeled and/or amplified nucleic acid molecules that correspond to hsa-miR-30c-5p/hsa-miR-186-5p. In some embodiments, a method described herein comprises producing labeled and/or amplified nucleic acid molecules that correspond to hsa-miR-516b-5p/hsa-miR-629-5p. In some embodiments, a method described herein comprises producing labeled and/or amplified nucleic acid molecules that correspond to hsa-miR-23a-3p/hsa-miR-93-5p. In some embodiments, a method described herein comprises producing labeled and/or amplified nucleic acid molecules that correspond to hsa-miR-127-3p/hsa-miR-485-5p. In some embodiments, a method described herein comprises producing labeled and/or amplified nucleic acid molecules that correspond to hsa-miR-155-5p/hsa-miR-451a. In some embodiments, a method described herein comprises producing labeled and/or amplified nucleic acid molecules that correspond to hsa-miR-30c-5p/hsa-miR-26b-5p. In some embodiments, a method described herein comprises producing labeled and/or amplified nucleic acid molecules that correspond to hsa-miR-4732-3p/hsa-miR-516b-5p. In some embodiments, a method described herein comprises producing labeled and/or amplified nucleic acid molecules that correspond to hsa-miR-7-5p/hsa-miR-941. In some embodiments, a method described herein comprises producing labeled and/or amplified nucleic acid molecules that correspond to hsa-miR-451a/hsa-miR-485-5p. In some embodiments, a method described herein comprises producing labeled and/or amplified nucleic acid molecules that correspond to hsa-miR-374b-5p/hsa-miR-885-3p. In some embodiments, a method described herein comprises producing labeled and/or amplified nucleic acid molecules that correspond to hsa-miR-516b-5p/hsa-miR-1307-3p. In some embodiments, a method described herein comprises producing labeled and/or amplified nucleic acid molecules that correspond to hsa-miR-516b-5p/hsa-miR-30d-5p. In some embodiments, a method described herein comprises producing labeled and/or amplified nucleic acid molecules that correspond to hsa-miR-3605-5p/hsa-miR-425-3p. In some embodiments, a method described herein comprises producing labeled and/or amplified nucleic acid molecules that correspond to hsa-miR-501-3p/hsa-miR-516b-5p. In some embodiments, a method described herein comprises producing labeled and/or amplified nucleic acid molecules that correspond to hsa-miR-125b-5p/hsa-miR-485-5p. In some embodiments, a method described herein comprises producing labeled and/or amplified nucleic acid molecules that correspond to hsa-miR-26b-5p/hsa-miR-485-5p. In some embodiments, a method described herein comprises producing labeled and/or amplified nucleic acid molecules that correspond to hsa-miR-30c-5p/hsa-miR-99a-5p. In some embodiments, a method described herein comprises producing labeled and/or amplified nucleic acid molecules that correspond to hsa-miR-516b-5p/hsa-miR-127-3p. In some embodiments, a method described herein comprises producing labeled and/or amplified nucleic acid molecules that correspond to hsa-miR-501-3p/hsa-miR-485-5p. In some embodiments, a method described herein comprises producing labeled and/or amplified nucleic acid molecules that correspond to hsa-miR-30c-5p/hsa-miR-451a. In some embodiments, a method described herein comprises producing labeled and/or amplified nucleic acid molecules that correspond to hsa-miR-21-5p/hsa-miR-485-5p. In some embodiments, a method described herein comprises producing labeled and/or amplified nucleic acid molecules that correspond to hsa-miR-516b-5p/hsa-miR-423-3p. In some embodiments, a method described herein comprises producing labeled and/or amplified nucleic acid molecules that correspond to hsa-miR-516b-5p/hsa-miR-146b-5p. In some embodiments, a method described herein comprises producing labeled and/or amplified nucleic acid molecules that correspond to hsa-miR-330-5p/hsa-miR-92b-5p. In some embodiments, a method described herein comprises producing labeled and/or amplified nucleic acid molecules that correspond to hsa-miR-4443/hsa-miR-485-5p. In some embodiments, a method described herein comprises producing labeled and/or amplified nucleic acid molecules that correspond to hsa-miR-30c-5p/hsa-let-7i-5p. In some embodiments, a method described herein comprises producing labeled and/or amplified nucleic acid molecules that correspond to hsa-miR-516b-5p/hsa-miR-425-5p. In some embodiments, a method described herein comprises producing labeled and/or amplified nucleic acid molecules that correspond to hsa-miR-516b-5p/hsa-miR-24-3p. In some embodiments, a method described herein comprises producing labeled and/or amplified nucleic acid molecules that correspond to hsa-miR-146b-5p/hsa-miR-485-5p. In some embodiments, a method described herein comprises producing labeled and/or amplified nucleic acid molecules that correspond to hsa-miR-760/hsa-miR-378g. In some embodiments, a method described herein comprises producing labeled and/or amplified nucleic acid molecules that correspond to hsa-miR-3615/hsa-miR-485-5p. In some embodiments, a method described herein comprises producing labeled and/or amplified nucleic acid molecules that correspond to hsa-miR-140-3p/hsa-miR-485-5p. In some embodiments, a method described herein comprises producing labeled and/or amplified nucleic acid molecules that correspond to hsa-miR-30c-5p/hsa-miR-125a-5p. In some embodiments, a method described herein comprises producing labeled and/or amplified nucleic acid molecules that correspond to hsa-miR-378c/hsa-miR-148a-5p. In some embodiments, a method described herein comprises producing labeled and/or amplified nucleic acid molecules that correspond to hsa-let-7b-5p/hsa-miR-485-5p. In some embodiments, a method described herein comprises producing labeled and/or amplified nucleic acid molecules that correspond to hsa-miR-516b-5p/hsa-miR-146a-5p. In some embodiments, a method described herein comprises producing labeled and/or amplified nucleic acid molecules that correspond to hsa-miR-516b-5p/hsa-miR-451a.

In some embodiments, a method described herein comprises producing labeled and/or amplified nucleic acid molecules that correspond to hsa-miR-140-3p/hsa-miR-516b-5p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-155-5p/hsa-miR-625-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-30c-5p/hsa-miR-27b-3p and at least one biomarker pair selected from the group consisting of hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-140-3p/hsa-miR-516b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-155-5p/hsa-miR-625-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-30c-5p/hsa-miR-27b-3p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-516b-5p/hsa-let-7b-5p, hsa-miR-516b-5p/hsa-miR-423-5p and hsa-miR-155-5p/hsa-let-7i-5p.

In some embodiments, a method described herein comprises producing labeled and/or amplified nucleic acid molecules that correspond to hsa-miR-516b-5p/hsa-let-7b-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-516b-5p/hsa-miR-423-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-155-5p/hsa-let-7i-5p and at least one biomarker pair selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a.

In some embodiments, a method described herein comprises producing labeled and/or amplified nucleic acid molecules that correspond to hsa-miR-516b-5p/hsa-let-7b-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-516b-5p/hsa-miR-423-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to hsa-miR-155-5p/hsa-let-7i-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to one or more of the panels of nucleic acid biomarkers listed in Table 6.

For example, in some embodiments, a method described herein comprises producing labeled and/or amplified nucleic acid molecules that correspond to hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, a method described herein comprises producing labeled and/or amplified nucleic acid molecules that correspond to hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, a method described herein comprises producing labeled and/or amplified nucleic acid molecules that correspond to hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, a method described herein comprises producing labeled and/or amplified nucleic acid molecules that correspond to hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; and hsa-miR-501-3p/hsa-miR-484.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, a method described herein comprises producing labeled and/or amplified nucleic acid molecules that correspond to hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; and hsa-miR-628-3p/hsa-miR-19b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, a method described herein comprises producing labeled and/or amplified nucleic acid molecules that correspond to hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p.

In some embodiments, a method described herein comprises producing labeled and/or amplified nucleic acid molecules that correspond to hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, a method described herein comprises producing labeled and/or amplified nucleic acid molecules that correspond to hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, a method described herein comprises producing labeled and/or amplified nucleic acid molecules that correspond to hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, a method described herein comprises producing labeled and/or amplified nucleic acid molecules that correspond to hsa-miR-7-5p/hsa-miR-485-5p; hsa-miR-30c-5p/hsa-miR-125a-5p; and hsa-miR-1-3p/hsa-miR-345-5p.

In some embodiments, a method described herein comprises producing labeled and/or amplified nucleic acid molecules that correspond to hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, a method described herein comprises producing labeled and/or amplified nucleic acid molecules that correspond to hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p.

In some embodiments, a method described herein comprises producing labeled and/or amplified nucleic acid molecules that correspond to hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1260b/hsa-mir-320c-2; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, a method described herein comprises producing labeled and/or amplified nucleic acid molecules that correspond to hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, a method described herein comprises producing labeled and/or amplified nucleic acid molecules that correspond to hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, a method described herein comprises producing labeled and/or amplified nucleic acid molecules that correspond to hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, a method described herein comprises producing labeled and/or amplified nucleic acid molecules that correspond to hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p; hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, a method described herein comprises producing labeled and/or amplified nucleic acid molecules that correspond to hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, a method described herein comprises producing labeled and/or amplified nucleic acid molecules that correspond to hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, a method described herein comprises producing labeled and/or amplified nucleic acid molecules that correspond to hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, a method described herein comprises producing labeled and/or amplified nucleic acid molecules that correspond to hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, a method described herein comprises producing labeled and/or amplified nucleic acid molecules that correspond to hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, a method described herein comprises producing labeled and/or amplified nucleic acid molecules that correspond to hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, a method described herein comprises producing labeled and/or amplified nucleic acid molecules that correspond to hsa-miR-7-5p/hsa-miR-485-5p;

hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-7-5p/hsa-miR-485-5p;

hsa-miR-221-5p/hsa-miR-760; hsa-miR-99b-3p/hsa-miR-340-5p; and hsa-miR-1306-3p/hsa-miR-150-3p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, a method described herein comprises producing labeled and/or amplified nucleic acid molecules that correspond to hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, a method described herein comprises producing labeled and/or amplified nucleic acid molecules that correspond to hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-330-5p/hsa-miR-223-3p; hsa-miR-7-5p/hsa-miR-485-5p; and hsa-miR-511-5p/hsa-miR-148a-5p.

In some embodiments, a method described herein comprises producing labeled and/or amplified nucleic acid molecules that correspond to hsa-miR-7-5p/hsa-miR-485-5p;

hsa-miR-1306-3p/hsa-miR-664a-5p; hsa-miR-30a-5p/hsa-miR-941; and hsa-miR-30c-5p/hsa-miR-27b-3p.

In some embodiments, this disclosure provides a method of determining a pregnant female's risk of developing placental dysfunction, wherein the biological sample is obtained between 17 and 29 weeks of gestational age at blood draw.

In some embodiments, this disclosure provides a method of determining a pregnant female's risk of developing placental dysfunction, wherein the biological sample is obtained between 19 and 27 weeks of gestational age at blood draw.

In some embodiments, this disclosure provides a method of determining a pregnant female's risk of developing placental dysfunction, wherein the biological sample is obtained between 17 and 36 weeks of gestational age at blood draw.

In some embodiments, this disclosure provides a method of determining a pregnant female's risk of developing placental dysfunction, wherein the biological sample is obtained more than 17, more than 18, more than 19, more than 20, more than 21, more than 22, more than 23, more than 24, more than 25, more than 26, more than 27, more than 28, more than 29, more than 30, more than 31, more than 32, more than 33, more than 34, or more than 35 weeks of gestational age at blood draw.

In some embodiments, this disclosure provides a method of determining a pregnant female's risk of developing placental dysfunction, wherein the biological sample is obtained between 119-153 days, 154-196 days, and/or 119-202 days.

In some embodiments, this disclosure provides a method of determining a pregnant female's risk of developing placental dysfunction, wherein the risk score is calculated based on a ratio of data values. In some embodiments, data transformation is applied before or after the ratio is determined.

In some embodiments, this disclosure provides a method of determining a pregnant female's risk of developing placental dysfunction, wherein the method determines the pregnant female's risk of developing placental dysfunction early in the pregnancy. In some embodiments, early pregnancy is between 119-153 days of gestation.

In other embodiments, this disclosure provides a method of determining a pregnant female's risk of developing placental dysfunction, wherein the method determines the pregnant female's risk of developing placental dysfunction later in the pregnancy. In some embodiments, late pregnancy is between 154-196 days of gestation.

In one aspect, the invention provides a method of determining a pregnant female's risk of developing placental dysfunction comprising producing labeled and/or amplified nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p. In some embodiments, a method described herein comprises producing labeled and/or amplified nucleic acid molecules that correspond to at least one, at least two, at least three, at least four, at least five, at least six, at least seven, at least eight, at least nine, at least ten, at least eleven, at least twelve, or at least 13 of the nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p.

In another aspect, the invention provides a method of determining a pregnant female's risk of developing placental dysfunction comprising producing labeled and/or amplified nucleic acid molecules selected from one of Tables 3, 4, or 5, in addition to producing labeled and/or amplified nucleic acid molecules selected from one of the panels of nucleic acid biomarkers listed in Table 6.

In some embodiments, a method described herein comprises producing labeled and/or amplified nucleic acid molecules that corresponds to hsa-miR-30c-5p. In some embodiments, a method described herein comprises hsa-miR-6842-3p. In some embodiments, a method described herein comprises hsa-miR-485-5p. In some embodiments, a method described herein comprises hsa-miR-331-3p. In some embodiments, a method described herein comprises hsa-miR-517a-3p-hsa-miR-517b-3p. In some embodiments, a method described herein comprises hsa-miR-516b-5p. In some embodiments, a method described herein comprises hsa-miR-760. In some embodiments, a method described herein comprises hsa-miR-6852-5p. In some embodiments, a method described herein comprises hsa-miR-1323. In some embodiments, a method described herein comprises hsa-miR-155-5p. In some embodiments, a method described herein comprises hsa-miR-6747-3p. In some embodiments, a method described herein comprises hsa-miR-181d-5p. In some embodiments, a method described herein comprises hsa-miR-4446-3p.

In some embodiments, a method described herein comprises producing labeled and/or amplified nucleic acid molecules that correspond to hsa-miR-485-5p and at least one biomarker selected from the group consisting of hsa-miR-7-5p, hsa-miR-501-3p, hsa-miR-363-3p, hsa-miR-182-5p, hsa-miR-127-3p, hsa-miR-140-3p, hsa-miR-26b-5p.

In some embodiments, a method described herein comprises producing labeled and/or amplified nucleic acid molecules that correspond to hsa-miR-485-5p and at least one pair biomarkers selected from the group consisting of hsa-miR-27b-3p, hsa-let-7i-5p, hsa-miR-99a-5p, hsa-miR-409-3p.

In some embodiments, a method described herein comprises producing labeled and/or amplified nucleic acid molecules that correspond to hsa-miR-485-5p and two or more biomarkers selected from Table 5.

In some embodiments, a method described herein comprises producing labeled and/or amplified nucleic acid molecules that correspond to hsa-miR-155-5p/hsa-let-7i-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises producing labeled and/or amplified nucleic acid molecules that correspond to one or more of the panels of nucleic acid biomarkers listed in Table 6.

For example, in some embodiments, a method described herein comprises producing labeled and/or amplified nucleic acid molecules that correspond to hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, a method described herein comprises producing labeled and/or amplified nucleic acid molecules that correspond to hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, a method described herein comprises producing labeled and/or amplified nucleic acid molecules that correspond to hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, a method described herein comprises producing labeled and/or amplified nucleic acid molecules that correspond to hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; and hsa-miR-501-3p/hsa-miR-484.

In some embodiments, a method described herein comprises producing labeled and/or amplified nucleic acid molecules that correspond to hsa-miR-7-5p/hsa-miR-485-5p;

hsa-miR-1306-3p/hsa-miR-664a-5p; hsa-miR-99b-3p/hsa-miR-760; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, a method described herein comprises producing labeled and/or amplified nucleic acid molecules that correspond to hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; and hsa-miR-628-3p/hsa-miR-19b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, a method described herein comprises producing labeled and/or amplified nucleic acid molecules that correspond to hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p;

hsa-miR-433-3p/hsa-miR-345-5p; hsa-miR-15b-5p/hsa-miR-197-3p; and hsa-miR-330-5p/hsa-miR-223-3p.

In some embodiments, a method described herein comprises producing labeled and/or amplified nucleic acid molecules that correspond to hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, a method described herein comprises producing labeled and/or amplified nucleic acid molecules that correspond to hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, a method described herein comprises producing labeled and/or amplified nucleic acid molecules that correspond to hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, a method described herein comprises producing labeled and/or amplified nucleic acid molecules that correspond to hsa-miR-7-5p/hsa-miR-485-5p; hsa-miR-30c-5p/hsa-miR-125a-5p; and hsa-miR-1-3p/hsa-miR-345-5p.

In some embodiments, a method described herein comprises producing labeled and/or amplified nucleic acid molecules that correspond to hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, a method described herein comprises producing labeled and/or amplified nucleic acid molecules that correspond to hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-7-5p/hsa-miR-485-5p;

hsa-miR-1306-3p/hsa-miR-664a-5p; hsa-miR-99b-3p/hsa-miR-760; and hsa-miR-760/hsa-miR-345-5p.

In some embodiments, a method described herein comprises producing labeled and/or amplified nucleic acid molecules that correspond to hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, a method described herein comprises producing labeled and/or amplified nucleic acid molecules that correspond to hsa-miR-7-5p/hsa-miR-485-5p; hsa-miR-1260b/hsa-mir-320c-2; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, a method described herein comprises producing labeled and/or amplified nucleic acid molecules that correspond to hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, a method described herein comprises producing labeled and/or amplified nucleic acid molecules that correspond to hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, a method described herein comprises producing labeled and/or amplified nucleic acid molecules that correspond to hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, a method described herein comprises producing labeled and/or amplified nucleic acid molecules that correspond to hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, a method described herein comprises producing labeled and/or amplified nucleic acid molecules that correspond to hsa-miR-7-5p/hsa-miR-485-5p;

hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, a method described herein comprises producing labeled and/or amplified nucleic acid molecules that correspond to hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, a method described herein comprises producing labeled and/or amplified nucleic acid molecules that correspond to hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, a method described herein comprises producing labeled and/or amplified nucleic acid molecules that correspond to hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, a method described herein comprises producing labeled and/or amplified nucleic acid molecules that correspond to hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, a method described herein comprises producing labeled and/or amplified nucleic acid molecules that correspond to hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, a method described herein comprises producing labeled and/or amplified nucleic acid molecules that correspond to hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, a method described herein comprises producing labeled and/or amplified nucleic acid molecules that correspond to hsa-miR-7-5p/hsa-miR-485-5p; hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, a method described herein comprises producing labeled and/or amplified nucleic acid molecules that correspond to hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-7-5p/hsa-miR-485-5p; hsa-miR-221-5p/hsa-miR-760; hsa-miR-99b-3p/hsa-miR-340-5p; and hsa-miR-1306-3p/hsa-miR-150-3p.

In some embodiments, a method described herein comprises producing labeled and/or amplified nucleic acid molecules that correspond to hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, a method described herein comprises producing labeled and/or amplified nucleic acid molecules that correspond to hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, a method described herein comprises producing labeled and/or amplified nucleic acid molecules that correspond to hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises producing labeled and/or amplified nucleic acid molecules that correspond to hsa-miR-330-5p/hsa-miR-223-3p; hsa-miR-7-5p/hsa-miR-485-5p; and hsa-miR-511-5p/hsa-miR-148a-5p.

In some embodiments, a method described herein comprises producing labeled and/or amplified nucleic acid molecules that correspond to hsa-miR-7-5p/hsa-miR-485-5p; hsa-miR-1306-3p/hsa-miR-664a-5p; hsa-miR-30a-5p/hsa-miR-941; and hsa-miR-30c-5p/hsa-miR-27b-3p.

In some embodiments, the invention provides a method of determining a pregnant female's risk of developing placental dysfunction, wherein the biological sample is obtained between 17 and 28 weeks of gestational age at blood draw.

In some embodiments, the invention provides a method of determining a pregnant female's risk of developing placental dysfunction, wherein the biological sample is obtained between 19 and 27 weeks of gestational age at blood draw.

In some embodiments, the invention provides a method of determining a pregnant female's risk of developing placental dysfunction, wherein the biological sample is obtained between 17 and 36 weeks of gestational age at blood draw.

In some embodiments, the invention provides a method of determining a pregnant female's risk of developing placental dysfunction, wherein the biological sample is obtained more than 17, more than 18, more than 19, more than 20, more than 21, more than 22, more than 23, more than 24, more than 25, more than 26, more than 27, more than 28, more than 29, more than 30, more than 31, more than 32, more than 33, more than 34, or more than 35 weeks of gestational age at blood draw.

In some embodiments, the invention provides a method of determining a pregnant female's risk of developing placental dysfunction, wherein the biological sample is obtained between 119-153 days. 154-196 days, and/or 119-202 days.

In one aspect, the invention provides a method for determining a pregnant female's risk of developing placental dysfunction later in the pregnancy comprising measuring the amount of two or more of the nucleic acid biomarkers selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941 in a biological sample obtained from the pregnant female, calculating a risk score based upon the measured amounts of the nucleic acid biomarkers to determine the pregnant female's risk of developing placental dysfunction, and providing a score corresponding to the pregnant female's risk of developing placental dysfunction.

In some embodiments, the invention provides a method of determining a pregnant female's risk of developing placental dysfunction later in the pregnancy comprising measuring two or more of the nucleic acid biomarkers selected from the group consisting of hsa-miR-1-3p, hsa-miR-181d-5p, hsa-miR-378d, hsa-miR-4433b-5p, and hsa-miR-27a-5p.

In some embodiments, the invention provides a method of determining a pregnant female's risk of developing placental dysfunction later in the pregnancy comprising measuring the amount of two or more of the nucleic acid biomarkers selected from the group consisting of hsa-miR-516b-5p and hsa-miR-30c-5p.

In some embodiments, the invention provides a method of determining a pregnant female's risk of developing placental dysfunction later in the pregnancy comprising measuring the amount of two or more of the nucleic acid biomarkers selected from the group consisting of hsa-miR-516b-5p, hsa-miR-30c-5p, hsa-miR-1-3p, and hsa-miR-331-3p.

In some embodiments, the invention provides a method of determining a pregnant female's risk of developing placental dysfunction later in the pregnancy comprising measuring the amount of two or more of the nucleic acid biomarkers selected from the group consisting of hsa-miR-516b-5p, hsa-miR-30c-5p, hsa-miR-1-3p, hsa-miR-331-3p, hsa-miR-485-5p, and hsa-miR-181d-5p.

In one aspect, the invention provides a method for determining a pregnant female's risk of developing placental dysfunction comprising measuring the amount of nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p in a biological sample obtained from the pregnant female, calculating a risk score based upon the measured amounts of the nucleic acid biomarkers to determine the pregnant female's risk of developing placental dysfunction, and providing a score corresponding to the pregnant female's risk of developing placental dysfunction. In some embodiments, a method described herein comprises measuring the amount of at least one, at least two, at least three, at least four, at least five, at least six, at least seven, at least eight, at least nine, at least ten, at least eleven, at least twelve, or at least 13 nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p.

In a different aspect, the invention provides a method for determining a pregnant female's risk of developing placental dysfunction comprising measuring the amount of nucleic acid molecules selected from one or more of the panels of biomarkers listed in Table 6 in a biological sample obtained from the pregnant female, calculating a risk score based upon the measured amounts of the nucleic acid biomarkers to determine the pregnant female's risk of developing placental dysfunction, and providing a score corresponding to the pregnant female's risk of developing placental dysfunction.

In some embodiments, a method for determining a pregnant female's risk of developing placental dysfunction comprises measuring a nucleic acid molecule that corresponds to hsa-miR-30c-5p. In some embodiments, a method described herein comprises hsa-miR-6842-3p. In some embodiments, a method described herein comprises hsa-miR-485-5p. In some embodiments, a method described herein comprises hsa-miR-331-3p. In some embodiments, a method described herein comprises hsa-miR-517a-3p-hsa-miR-517b-3p. In some embodiments, a method described herein comprises hsa-miR-516b-5p. In some embodiments, a method described herein comprises hsa-miR-760. In some embodiments, a method described herein comprises hsa-miR-6852-5p. In some embodiments, a method described herein comprises hsa-miR-1323. In some embodiments, a method described herein comprises hsa-miR-155-5p. In some embodiments, a method described herein comprises hsa-miR-6747-3p. In some embodiments, a method described herein comprises hsa-miR-181d-5p. In some embodiments, a method described herein comprises hsa-miR-4446-3p.

In some embodiments, a method described herein comprises measuring nucleic acid molecules that correspond to hsa-miR-485-5p and at least one biomarker selected from the group consisting of hsa-miR-7-5p, hsa-miR-501-3p, hsa-miR-363-3p, hsa-miR-182-5p, hsa-miR-127-3p, hsa-miR-140-3p, hsa-miR-26b-5p.

In some embodiments, a method described herein comprises measuring nucleic acid molecules that correspond to hsa-miR-485-5p and at least one pair biomarkers selected from the group consisting of hsa-miR-27b-3p, hsa-let-7i-5p, hsa-miR-99a-5p, hsa-miR-409-3p.

In some embodiments, a method described herein comprises measuring nucleic acid molecules that correspond to hsa-miR-485-5p and two or more biomarkers selected from Table 5.

In some embodiments, a method described herein comprises measuring nucleic acid molecules that correspond to hsa-miR-155-5p/hsa-let-7i-5p and at least one biomarker selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941.

In some embodiments, a method described herein comprises measuring nucleic acid molecules that correspond to one or more of the panels of nucleic acid biomarkers listed in Table 6.

For example, in some embodiments, a method described herein comprises measuring nucleic acid molecules that correspond to hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p.

In some embodiments, a method described herein comprises measuring nucleic acid molecules that correspond to hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p.

In some embodiments, a method described herein comprises measuring nucleic acid molecules that correspond to hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p.

In some embodiments, a method described herein comprises measuring nucleic acid molecules that correspond to hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p.

In some embodiments, a method described herein comprises measuring nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760.

In some embodiments, a method described herein comprises measuring nucleic acid molecules that correspond to hsa-miR-7-5p/hsa-miR-485-5p; hsa-miR-1306-3p/hsa-miR-664a-5p; and hsa-miR-501-3p/hsa-miR-484.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p.

In some embodiments, a method described herein comprises measuring nucleic acid molecules that correspond to hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p.

In some embodiments, a method described herein comprises measuring nucleic acid molecules that correspond to hsa-miR-7-5p/hsa-miR-485-5p; hsa-miR-1306-3p/hsa-miR-664a-5p; and hsa-miR-628-3p/hsa-miR-19b-3p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760.

In some embodiments, a method described herein comprises measuring nucleic acid molecules that correspond to hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p.

In some embodiments, a method described herein comprises measuring nucleic acid molecules that correspond to hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760.

In some embodiments, a method described herein comprises measuring nucleic acid molecules that correspond to hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p.

In some embodiments, a method described herein comprises measuring nucleic acid molecules that correspond to hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p.

In some embodiments, a method described herein comprises measuring nucleic acid molecules that correspond to hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p.

In some embodiments, a method described herein comprises measuring nucleic acid molecules that correspond to hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p.

In some embodiments, a method described herein comprises measuring nucleic acid molecules that correspond to hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760.

In some embodiments, a method described herein comprises measuring nucleic acid molecules that correspond to hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p.

In some embodiments, a method described herein comprises measuring nucleic acid molecules that correspond to hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p.

In some embodiments, a method described herein comprises measuring nucleic acid molecules that correspond to hsa-miR-7-5p/hsa-miR-485-5p; hsa-miR-30c-5p/hsa-miR-125a-5p; and hsa-miR-1-3p/hsa-miR-345-5p.

In some embodiments, a method described herein comprises measuring nucleic acid molecules that correspond to hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p.

In some embodiments, a method described herein comprises measuring nucleic acid molecules that correspond to hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p.

In some embodiments, a method described herein comprises measuring nucleic acid molecules that correspond to hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p.

In some embodiments, a method described herein comprises measuring nucleic acid molecules that correspond to hsa-miR-1306-3p/hsa-miR-664a-5p; hsa-miR-378g/hsa-miR-485-5p; hsa-miR-628-3p/hsa-miR-19b-3p; and hsa-miR-221-5p/hsa-miR-760.

In some embodiments, a method described herein comprises measuring nucleic acid molecules that correspond to hsa-miR-7-5p/hsa-miR-485-5p; hsa-miR-1260b/hsa-mir-320c-2; and hsa-miR-433-3p/hsa-miR-345-5p.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; hsa-miR-1-3p/hsa-miR-345-5p; and hsa-miR-760/hsa-miR-345-5p.

In some embodiments, a method described herein comprises measuring nucleic acid molecules that correspond to hsa-miR-1306-3p/hsa-miR-664a-5p; hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p.

In some embodiments, a method described herein comprises measuring nucleic acid molecules that correspond to hsa-miR-7-5p/hsa-miR-485-5p; hsa-miR-1306-3p/hsa-miR-1224-5p; hsa-miR-99b-3p/hsa-miR-760; and hsa-miR-543/hsa-miR-345-5p.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-7-5p.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-1-3p/hsa-miR-345-5p.

In some embodiments, a method described herein comprises measuring nucleic acid molecules that correspond to hsa-miR-98-5p/hsa-miR-485-5p; hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-1-3p/hsa-miR-345-5p; and hsa-miR-628-3p/hsa-miR-19b-3p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-323b-3p/hsa-mir-320c-2.

In some embodiments, a method described herein comprises measuring nucleic acid molecules that correspond to hsa-miR-7-5p/hsa-miR-485-5p; hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-99b-3p/hsa-miR-760; and hsa-miR-7-5p/hsa-let-7c-5p.

In some embodiments, a method described herein comprises measuring nucleic acid molecules that correspond to hsa-miR-30c-5p/hsa-miR-27b-3p; hsa-miR-7-5p/hsa-miR-485-5p; hsa-miR-1260b/hsa-mir-320c-2; and hsa-miR-433-3p/hsa-miR-345-5p.

In some embodiments, a method described herein comprises measuring nucleic acid molecules that correspond to hsa-miR-7-5p/hsa-miR-485-5p; hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p.

In some embodiments, a method described herein comprises measuring nucleic acid molecules that correspond to hsa-miR-7-5p/hsa-miR-485-5p; hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p.

In some embodiments, a method described herein comprises measuring nucleic acid molecules that correspond to hsa-miR-98-5p/hsa-miR-485-5p; hsa-miR-1306-3p/hsa-miR-664a-5p; hsa-miR-99b-3p/hsa-miR-485-5p; and hsa-miR-221-5p/hsa-miR-197-3p.

In some embodiments, a method described herein comprises measuring nucleic acid molecules that correspond to hsa-miR-30c-5p/hsa-miR-27b-3p; hsa-miR-221-5p/hsa-miR-197-3p; hsa-miR-1306-3p/hsa-miR-150-3p; and hsa-miR-15b-5p/hsa-miR-197-3p.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-miR-21-5p.

In some embodiments, a method described herein comprises measuring nucleic acid molecules that correspond to hsa-miR-7-5p/hsa-miR-485-5p; hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p.

In some embodiments, a method described herein comprises measuring nucleic acid molecules that correspond to hsa-miR-30c-5p/hsa-miR-27b-3p; hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-30c-5p/hsa-let-7g-5p.

In some embodiments, a method described herein comprises measuring nucleic acid molecules that correspond to hsa-miR-7-5p/hsa-miR-485-5p; hsa-miR-1306-3p/hsa-miR-1224-5p; hsa-miR-142-5p/hsa-miR-485-5p; and hsa-miR-221-5p/hsa-miR-197-3p.

In some embodiments, a method described herein comprises measuring nucleic acid molecules that correspond to hsa-miR-98-5p/hsa-miR-485-5p; hsa-miR-330-5p/hsa-miR-550a-3-5p-hsa-miR-550a-5p; and hsa-miR-30c-5p/hsa-miR-625-3p.

In some embodiments, a method described herein comprises measuring nucleic acid molecules that correspond to hsa-miR-7-5p/hsa-miR-485-5p; hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p.

In some embodiments, a method described herein comprises measuring nucleic acid molecules that correspond to hsa-miR-1306-3p/hsa-miR-664a-5p; hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises measuring nucleic acid molecules that correspond to hsa-miR-7-5p/hsa-miR-485-5p; hsa-miR-221-5p/hsa-miR-760; hsa-miR-99b-3p/hsa-miR-340-5p; and hsa-miR-1306-3p/hsa-miR-150-3p.

In some embodiments, a method described herein comprises measuring nucleic acid molecules that correspond to hsa-miR-7-5p/hsa-miR-485-5p; hsa-miR-330-5p/hsa-miR-550a-3-5p-hsa-miR-550a-5p; and hsa-miR-323b-3p/hsa-mir-320c-2.

In some embodiments, a method described herein comprises measuring nucleic acid molecules that correspond to hsa-miR-1306-3p/hsa-miR-664a-5p; hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p.

In some embodiments, a method described herein comprises measuring nucleic acid molecules that correspond to hsa-miR-221-5p/hsa-miR-760; hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p.

In some embodiments, a method described herein comprises measuring nucleic acid molecules that correspond to hsa-miR-330-5p/hsa-miR-223-3p; hsa-miR-7-5p/hsa-miR-485-5p; and hsa-miR-511-5p/hsa-miR-148a-5p.

In some embodiments, a method described herein comprises measuring nucleic acid molecules that correspond to hsa-miR-7-5p/hsa-miR-485-5p; hsa-miR-1306-3p/hsa-miR-664a-5p; hsa-miR-30a-5p/hsa-miR-941; and hsa-miR-30c-5p/hsa-miR-27b-3p.

In one aspect, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers selected from the group consisting of hsa-miR-516b-5p, hsa-miR-30c-5p, hsa-miR-1-3p, hsa-miR-331-3p, hsa-miR-485-5p, hsa-miR-181d-5p, hsa-miR-23a-3p, hsa-miR-378d, hsa-miR-155-5p, hsa-miR-99b-3p, hsa-miR-1249-3p, hsa-miR-142-3p, hsa-miR-4433b-5p, hsa-miR-1273h-3p, hsa-miR-18a-3p, hsa-miR-4746-5p, hsa-miR-27a-5p, hsa-miR-654-3p, hsa-miR-7-5p, and hsa-miR-941 in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

In another aspect, the invention provides a method of determining a pregnant female's risk of developing placental dysfunction later in the pregnancy comprising producing labeled and/or amplified nucleic acid molecules, for example, amplified labeled nucleic acid molecules, that correspond to one or more of the panels of biomarkers listed in Table 6, in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-1-3p, hsa-miR-181d-5p, hsa-miR-378d, hsa-miR-4433b-5p, and/or hsa-miR-27a-5p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-516b-5p and/or hsa-miR-30c-5p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-516b-5p, hsa-miR-30c-5p, hsa-miR-1-3p, and/or hsa-miR-331-3p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond tohsa-miR-516b-5p, hsa-miR-30c-5p, hsa-miR-1-3p, and/or hsa-miR-331-3p.

In one aspect, the invention provides a method for determining a pregnant female's risk of developing placental dysfunction later in the pregnancy comprising measuring the amount of a pair of biomarkers selected from the group consisting of hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a in a biological sample obtained from the pregnant female, calculating a risk score based upon the measured amounts of the nucleic acid biomarker pair to determine the pregnant female's risk of developing placental dysfunction, and providing a score corresponding to the pregnant female's risk of developing placental dysfunction.

In some embodiments, the invention provides a method for determining a pregnant female's risk of developing placental dysfunction later in the pregnancy comprising measuring the amount of a pair of biomarkers that correspond to hsa-miR-30c-5p/hsa-miR-27b-3p, hsa-miR-516b-5p/hsa-miR-150-5p, hsa-miR-30c-5p/hsa-miR-16-5p, hsa-miR-30c-5p/hsa-miR-30a-5p, hsa-miR-516b-5p/hsa-miR-92a-3p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-23a-3p/hsa-miR-27b-3p, hsa-miR-30a-5p/hsa-miR-485-5p, hsa-miR-516b-5p/hsa-let-7a-5p, hsa-miR-516b-5p/hsa-miR-222-3p, hsa-miR-516b-5p/hsa-let-7f-5p, hsa-miR-485-5p/hsa-miR-183-5p, hsa-miR-4433b-5p/hsa-miR-516b-5p, hsa-miR-516b-5p/hsa-miR-10b-5p, hsa-miR-516b-5p/hsa-miR-328-3p, hsa-miR-155-5p/hsa-miR-30a-5p, hsa-miR-516b-5p/hsa-miR-128-3p, hsa-miR-516b-5p/hsa-miR-744-5p, hsa-miR-516b-5p/hsa-miR-629-5p, hsa-miR-23a-3p/hsa-miR-93-5p, hsa-miR-3605-5p/hsa-miR-425-3p, hsa-miR-21-5p/hsa-miR-485-5p, hsa-miR-516b-5p/hsa-miR-24-3p, and/or hsa-miR-378c/hsa-miR-148a-5p.

In some embodiments, the invention provides a method for determining a pregnant female's risk of developing placental dysfunction later in the pregnancy comprising measuring the amount of a pair of biomarkers that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, and/or hsa-miR-484/hsa-miR-516b-5p.

In some embodiments, the invention provides a method for determining a pregnant female's risk of developing placental dysfunction later in the pregnancy comprising measuring the amount of a pair of biomarkers that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, and/or hsa-miR-30c-5p/hsa-miR-7-5p.

In some embodiments, the invention provides a method for determining a pregnant female's risk of developing placental dysfunction later in the pregnancy comprising measuring the amount of a pair of biomarkers that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, and/or hsa-miR-485-5p/hsa-miR-516b-5p.

In one aspect, the invention provides a method for determining a pregnant female's risk of developing placental dysfunction later in the pregnancy comprising producing labeled and/or amplified nucleic acid molecules, for example, amplified labeled nucleic acid molecules, that correspond to a pair of biomarkers selected from the group consisting hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, hsa-miR-485-5p/hsa-miR-516b-5p, hsa-miR-7-5p/hsa-miR-485-5p, hsa-miR-516b-5p/hsa-let-7g-5p, hsa-miR-30c-5p/hsa-miR-16-5p, hsa-miR-516b-5p/hsa-miR-423-5p, hsa-miR-30c-5p/hsa-miR-30a-5p, hsa-miR-516b-5p/hsa-miR-92a-3p, hsa-miR-186-5p/hsa-miR-516b-5p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-516b-5p/hsa-miR-126-3p, hsa-miR-99b-3p/hsa-miR-486-3p, hsa-miR-6741-5p/hsa-miR-760, hsa-miR-20a-5p/hsa-miR-485-5p, hsa-miR-23a-3p/hsa-miR-27b-3p, hsa-miR-516b-5p/hsa-miR-151a-3p, hsa-miR-30a-5p/hsa-miR-485-5p, hsa-miR-516b-5p/hsa-let-7a-5p, hsa-miR-30c-5p/hsa-miR-27a-3p, hsa-miR-485-5p/hsa-miR-7-5p, hsa-miR-516b-5p/hsa-miR-222-3p, hsa-miR-23a-3p/hsa-miR-30a-5p, hsa-miR-433-3p/hsa-miR-345-5p, hsa-miR-516b-5p/hsa-let-7f-5p, hsa-miR-485-5p/hsa-miR-183-5p, hsa-miR-516b-5p/hsa-miR-185-5p, hsa-let-7i-5p/hsa-miR-485-5p, hsa-miR-4433b-5p/hsa-miR-516b-5p, hsa-miR-516b-5p/hsa-miR-191-5p, hsa-miR-516b-5p/hsa-miR-3615, hsa-miR-100-5p/hsa-miR-516b-5p, hsa-miR-516b-5p/hsa-miR-10b-5p, hsa-miR-485-5p/hsa-miR-27a-3p, hsa-miR-99a-5p/hsa-miR-485-5p, hsa-miR-99b-3p/hsa-miR-6852-5p, hsa-miR-363-3p/hsa-miR-485-5p, hsa-miR-516b-5p/hsa-miR-484, hsa-miR-516b-5p/hsa-let-7i-5p, hsa-miR-23a-3p/hsa-miR-181a-5p, hsa-miR-7-5p/hsa-miR-516b-5p, hsa-miR-516b-5p/hsa-miR-328-3p, hsa-miR-181a-5p/hsa-miR-485-5p, hsa-miR-155-5p/hsa-miR-30a-5p, hsa-miR-155-5p/hsa-let-7i-5p, hsa-miR-98-5p/hsa-miR-485-5p, hsa-miR-485-5p/hsa-miR-127-3p, hsa-miR-320b/hsa-miR-485-5p, hsa-miR-516b-5p/hsa-miR-25-3p, hsa-miR-516b-5p/hsa-miR-148a-3p, hsa-miR-516b-5p/hsa-miR-128-3p, hsa-miR-516b-5p/hsa-miR-320a, hsa-miR-516b-5p/hsa-miR-744-5p, hsa-miR-30c-5p/hsa-miR-186-5p, hsa-miR-516b-5p/hsa-miR-629-5p, hsa-miR-23a-3p/hsa-miR-93-5p, hsa-miR-127-3p/hsa-miR-485-5p, hsa-miR-155-5p/hsa-miR-451a, hsa-miR-30c-5p/hsa-miR-26b-5p, hsa-miR-4732-3p/hsa-miR-516b-5p, hsa-miR-7-5p/hsa-miR-941, hsa-miR-451a/hsa-miR-485-5p, hsa-miR-374b-5p/hsa-miR-885-3p, hsa-miR-516b-5p/hsa-miR-1307-3p, hsa-miR-516b-5p/hsa-miR-30d-5p, hsa-miR-3605-5p/hsa-miR-425-3p, hsa-miR-501-3p/hsa-miR-516b-5p, hsa-miR-125b-5p/hsa-miR-485-5p, hsa-miR-26b-5p/hsa-miR-485-5p, hsa-miR-30c-5p/hsa-miR-99a-5p, hsa-miR-516b-5p/hsa-miR-127-3p, hsa-miR-501-3p/hsa-miR-485-5p, hsa-miR-30c-5p/hsa-miR-451a, hsa-miR-21-5p/hsa-miR-485-5p, hsa-miR-516b-5p/hsa-miR-423-3p, hsa-miR-516b-5p/hsa-miR-146b-5p, hsa-miR-330-5p/hsa-miR-92b-5p, hsa-miR-4443/hsa-miR-485-5p, hsa-miR-30c-5p/hsa-let-7i-5p, hsa-miR-516b-5p/hsa-miR-425-5p, hsa-miR-516b-5p/hsa-miR-24-3p, hsa-miR-146b-5p/hsa-miR-485-5p, hsa-miR-760/hsa-miR-378g, hsa-miR-3615/hsa-miR-485-5p, hsa-miR-140-3p/hsa-miR-485-5p, hsa-miR-30c-5p/hsa-miR-125a-5p, hsa-miR-378c/hsa-miR-148a-5p, hsa-let-7b-5p/hsa-miR-485-5p, hsa-miR-516b-5p/hsa-miR-146a-5p, and hsa-miR-516b-5p/hsa-miR-451a in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction. in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-30c-5p/hsa-miR-27b-3p, hsa-miR-516b-5p/hsa-miR-150-5p, hsa-miR-30c-5p/hsa-miR-16-5p, hsa-miR-30c-5p/hsa-miR-30a-5p, hsa-miR-516b-5p/hsa-miR-92a-3p, hsa-miR-155-5p/hsa-miR-27b-3p, hsa-miR-30e-5p/hsa-miR-516b-5p, hsa-miR-516b-5p/hsa-let-7d-5p, hsa-miR-516b-5p/hsa-miR-103a-3p, hsa-miR-23a-3p/hsa-miR-27b-3p, hsa-miR-30a-5p/hsa-miR-485-5p, hsa-miR-516b-5p/hsa-let-7a-5p, hsa-miR-516b-5p/hsa-miR-222-3p, hsa-miR-516b-5p/hsa-let-7f-5p, hsa-miR-485-5p/hsa-miR-183-5p, hsa-miR-4433b-5p/hsa-miR-516b-5p, hsa-miR-516b-5p/hsa-miR-10b-5p, hsa-miR-516b-5p/hsa-miR-328-3p, hsa-miR-155-5p/hsa-miR-30a-5p, hsa-miR-516b-5p/hsa-miR-128-3p, hsa-miR-516b-5p/hsa-miR-744-5p, hsa-miR-516b-5p/hsa-miR-629-5p, hsa-miR-23a-3p/hsa-miR-93-5p, hsa-miR-3605-5p/hsa-miR-425-3p, hsa-miR-21-5p/hsa-miR-485-5p, hsa-miR-516b-5p/hsa-miR-24-3p, and/or hsa-miR-378c/hsa-miR-148a-5p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, and/or hsa-miR-484/hsa-miR-516b-5p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, and/or hsa-miR-30c-5p/hsa-miR-7-5p.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-140-3p/hsa-miR-516b-5p, hsa-miR-155-5p/hsa-miR-625-3p, hsa-miR-30c-5p/hsa-miR-27b-3p, hsa-miR-25-5p/hsa-miR-516b-5p, hsa-miR-484/hsa-miR-516b-5p, hsa-miR-516b-5p/hsa-let-7b-5p, hsa-miR-30c-5p/hsa-miR-7-5p, hsa-miR-516b-5p/hsa-miR-486-5p, hsa-miR-516b-5p/hsa-miR-150-5p, and/or hsa-miR-485-5p/hsa-miR-516b-5p.

In one aspect, the invention provides a method for treating or preventing placental dysfunction in a pregnant female, the method comprising (a) detecting a panel of isolated nucleic acid biomarkers in a biological sample obtained from a pregnant female, wherein at least one of the isolated nucleic acid biomarkers is selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p; (b) calculating a risk score based upon the measured levels of the isolated nucleic acid biomarkers; and (c) administering one or more therapies to the pregnant female based on the risk score, to treat or prevent placental dysfunction. In some embodiments, a method described herein comprises detecting at least one, at least two, at least three, at least four, at least five, at least six, at least seven, at least eight, at least nine, at least ten, at least eleven, at least twelve, or at least 13 of the nucleic acid molecules selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p.

In some embodiments, a method described herein comprises detecting a nucleic acid molecule that corresponds to hsa-miR-30c-5p. In some embodiments, a method described herein comprises hsa-miR-6842-3p. In some embodiments, a method described herein comprises hsa-miR-485-5p. In some embodiments, a method described herein comprises hsa-miR-331-3p. In some embodiments, a method described herein comprises hsa-miR-517a-3p-hsa-miR-517b-3p. In some embodiments, a method described herein comprises hsa-miR-516b-5p. In some embodiments, a method described herein comprises hsa-miR-760. In some embodiments, a method described herein comprises hsa-miR-6852-5p. In some embodiments, a method described herein comprises hsa-miR-1323. In some embodiments, a method described herein comprises hsa-miR-155-5p. In some embodiments, a method described herein comprises hsa-miR-6747-3p. In some embodiments, a method described herein comprises hsa-miR-181d-5p. In some embodiments, a method described herein comprises hsa-miR-4446-3p.

In some embodiments, a method described herein comprises detecting a nucleic acid molecule that corresponds to hsa-miR-485-5p and at least one biomarker selected from the group consisting of hsa-miR-7-5p, hsa-miR-501-3p, hsa-miR-363-3p, hsa-miR-182-5p, hsa-miR-127-3p, hsa-miR-140-3p, hsa-miR-26b-5p.

In some embodiments, a method described herein comprises detecting a nucleic acid molecule that corresponds to a nucleic acid molecule that corresponds to hsa-miR-485-5p and at least one pair biomarkers selected from the group consisting of hsa-miR-27b-3p, hsa-let-7i-5p, hsa-miR-99a-5p, hsa-miR-409-3p.

In some embodiments, a method described herein comprises detecting a nucleic acid molecule that corresponds to a nucleic acid molecule that corresponds to hsa-miR-485-5p and two or more biomarkers selected from Table 5.

In some embodiments of methods of the invention, the risk score is calculated based on a ratio of data values. In some embodiments of methods of the invention, data transformation is applied before or after the ratio is determined.

The quantitation of biomarkers in a biological sample can be determined, without limitation, by the methods described above as well as any other method known in the art. The quantitative data thus obtained is then subjected to an analytic classification process. In such a process, the raw data is manipulated according to an algorithm, where the algorithm has been pre-defined by a training set of data, for example as described in the examples provided herein, including establishing a binary decision tree. An algorithm can utilize the training set of data provided herein, or can utilize the guidelines provided herein to generate an algorithm with a different set of data.

An analytic classification process can use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve (AUC) measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.5, at least about 0.55, at least about 0.6, at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each biomarker, usually in triplicate or in multiple triplicates. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246 (1964). The data are then input into a predictive model, which will classify the sample according to the state. The resulting information can be communicated to a patient or health care provider.

To generate a predictive model for determining the risk of developing placental dysfunction later in the pregnancy a robust data set, comprising known control samples and samples corresponding to the birth classification of interest is used in a training set. A sample size can be selected using generally accepted criteria. As discussed above, different statistical methods can be used to obtain a highly accurate predictive model.

In one embodiment, hierarchical clustering is performed in the derivation of a predictive model, where the Pearson correlation is employed as the clustering metric. One approach is to consider a given birth dataset as a “learning sample” in a problem of “supervised learning.” CART is a standard in applications to medicine (Singer, Recursive Partitioning in the Health Sciences, Springer (1999)) and can be modified by transforming any qualitative features to quantitative features; sorting them by attained significance levels, evaluated by sample reuse methods for Hotelling's T2 statistic; and suitable application of the lasso method. Problems in prediction are turned into problems in regression without losing sight of prediction, indeed by making suitable use of the Gini criterion for classification in evaluating the quality of regressions.

This approach led to what is termed FlexTree (Huang, Proc. Nat. Acad. Sci. U.S.A 101:10529-10534 (2004)). FlexTree performs very well in simulations and when applied to multiple forms of data and is useful for practicing the claimed methods. Software automating Flex Tree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. USA. 101 (29): 10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

In one embodiment, a binary tree analysis is used to determine a risk score for placental dysfunction based on one or more of the panels of biomarkers listed in Table 6. The binary tree analysis can efficiently deal with large, complicated datasets. When a sample size is large enough, study data can be divided into training and validation datasets. The training dataset can be used to build a decision tree model as described below and a validation dataset can be used to decide on the appropriate tree size needed to achieve an optimal final model. The final model may consider certain variables about a population being assessed to weigh or order nodes from which risk scores are assigned to subjects. Variables in a population that would cause a reweighting and/or reordering of the nodes include demographic and clinical maternal factors, such as ethnicity, race, BMI, average maternal age, and obstetric history (e.g., parity and gravidity). Nevertheless, the predictive power of the panel of biomarkers will still remain.

The binary decision tree analysis can classify a population into branch-like segments that resembly a tree with branches that terminate in nodes. At each branch point leading to a node, an analysis is performed in which a reversal score derived from a ratio of measured miRNA (e.g., hsa-miR-98-5p/hsa-miR-485-5p) is compared to a cut off value. The cut off value represents the optimal point separating cases (a patent with placental dysfunction) from controls (a patient without placental dysfunction). The cut off value can be assay dependent. Binary decision trees can be modeled from the data presented in Table 7 and described in Example 3.

In a binary decision tree panel, a first reversal is used to separate all subjects into 2 groups. This can be referred to as a first layer. If classification of either of the two groups produced by the first layer is significantly improved by applying another reversal, the second layer can provide a subgrouping based on additional reversals. If classification of any of the groups produced by the second layer can be significantly improved by applying an additional reversal, the third layer provides subgrouping based on these additional reversals.

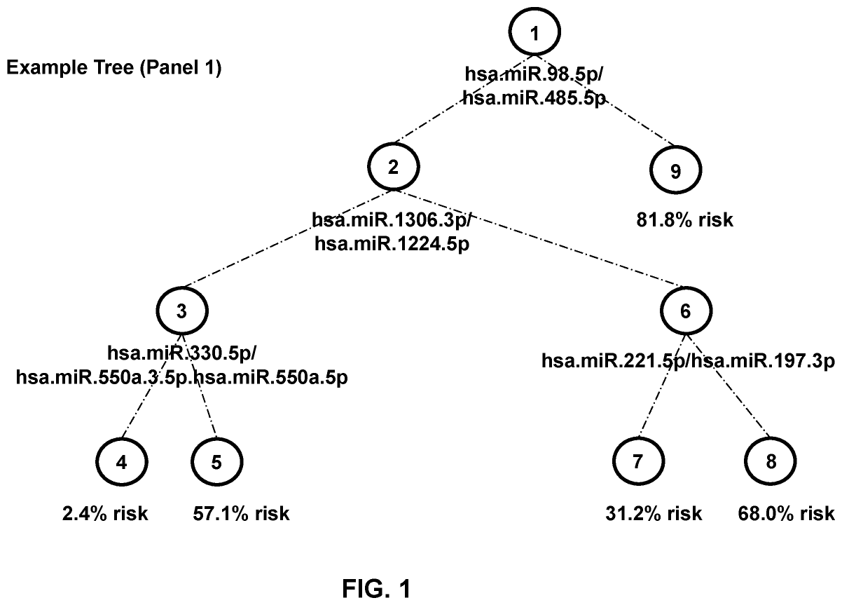

Each subject ends up in exactly 1 terminal node (subgroup that is not further split). Subjects can be assigned classification probabilities (risk scores) based on their terminal node. The order of reversals in a single row does imply position within a tree: top down, left to right. The first level of each tree has one binary split. The second level can have up to 2 binary splits. The third level can have up to 4 binary splits, e.g., see FIGS. 1-4.

All known performance statistics can be derived from the frequency-weighted average of the stratification in terminal nodes. Risk scores can be assigned to an individual subject in any particular node based on the proportion of cases (subjects with placental dysfunctionamongst all subjects assigned to that node in the clinical validation set.

Another approach is that of nearest shrunken centroids (Tibshirani, Proc. Natl. Acad. Sci. U.S.A 99:6567-72 (2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition the Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of biomarker, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

Survival analyses are commonly used to understand time to occurrence of an event of interest such as birth or death. Commonly, the Kaplan-Meier estimator is used to estimate the survival function, while Cox proportional hazards models are used to estimate the effects of covariates on the hazard of event occurrence. These models conventionally assume that survival time is based on risk of exactly one type of event. However a competing risk for a different event may be present that either hinders the observation of an event of interest or modifies the chance that this event occurs. Conventional methods may be inappropriate in the presence of competing risks. Alternative methods appropriate for analysis of competing risks either assess competing hazards in subdistribution hazards models or cause-specific modified Cox proportional hazards models; or estimate cumulative incidence over competing events (Jason P. Fine & Robert J. Gray. Journal of the American Statistical Association Vol. 94, Issue 446, 1999. A Proportional Hazards Model for the Subdistribution of a Competing Risk).

In the development of a predictive model, it can be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, linear discriminant analysis, recursive feature elimination, a prediction analysis of microarray, a logistic regression, a CART algorithm, a FlexTree algorithm, a LART algorithm, a random forest algorithm, a MART algorithm, and machine learning algorithms. Various methods are used in a training model. The selection of a subset of markers can be for a forward selection or a backward selection of a marker subset. The number of markers can be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

The biomarkers of the invention, which have been identified in the invention disclosed herein as being useful for predicting placental dysfunction, are known in the art and are readily available in public databases. For example, the human microRNAs disclosed herein as biomarkers useful for determining a pregnant female's risk of developing placental dysfunction are available in mirBase (mirbase.org). The naming convention for microRNAs generally uses “−” in the name of the microRNA, for example, hsa-miR-423-3p. It is noted that in some instances herein a shortened nomenclature is used, in which the “−” is replaced with “.” such as “hsa.miR.423.3p” instead of “hsa-miR-423-3p.” A person skilled in the art will readily understand the nomenclature commonly used for microRNAs and will appreciate that the microRNAs disclosed herein are readily available in public databases. Biomarker pairs are generally denoted herein as a pair separated by a “/”, for example, hsa-miR-516b-5p/hsa-miR-485-5p.

In yet another aspect, the invention provides kits for determining a pregnant female's risk of developing placental dysfunction later in the pregnancy. The kit can include one or more agents for detection of biomarkers, a container for holding a biological sample isolated from a pregnant female; and printed instructions for reacting agents with the biological sample or a portion of the biological sample to detect the presence or amount of the isolated biomarkers in the biological sample. The agents can be packaged in separate containers. The kit can further comprise one or more control reference samples and reagents for performing an immunoassay.

The kit can comprise one or more containers for compositions or reagents contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods for determining a pregnant female's risk of developing placental dysfunction later in the pregnancy.

In yet another aspect, the invention provides a method of treating or preventing placental dysfunction in a pregnant female comprising (a) detecting a panel of isolated nucleic acid biomarkers in a biological sample obtained from a pregnant female, wherein at least one of the isolated nucleic acid biomarkers is selected from the group consisting of hsa-miR-30c-5p, hsa-miR-6842-3p, hsa-miR-485-5p, hsa-miR-331-3p, hsa-miR-517a-3p-hsa-miR-517b-3p, hsa-miR-516b-5p, hsa-miR-760, hsa-miR-6852-5p, hsa-miR-1323, hsa-miR-155-5p, hsa-miR-6747-3p, hsa-miR-181d-5p, and hsa-miR-4446-3p; (b) calculating a risk score based upon the measured levels of the isolated nucleic acid biomarkers; and (c) administering one or more therapies to the pregnant female based on the risk score, to treat or prevent placental dysfunction.

In another aspect, this disclosure provides a method of treating or preventing placental dysfunction in a pregnant female, the method comprising: (a) detecting the panel of isolated nucleic acid biomarkers of any one or more of the panels of nucleic acid biomarkers listed in Table 6 in a biological sample obtained from a pregnant female; (b) calculating a risk score based upon measured levels of the isolated nucleic acid biomarkers; and (c) administering one or more therapies to the pregnant female based on the risk score, to treat or prevent placental dysfunction. In some embodiments, the method further comprises a step of determining gestational age at blood draw (GABD). In some embodiments, determining GABD is performed before the detecting step.

In a different aspect, this disclosure provides a method of treating or preventing placental dysfunction in a pregnant female, the method comprising: (a) detecting the composition of labeled and/or amplified nucleic acid molecules of any one or more of the panels of nucleic acid biomarkers listed in Table 6 from a biological sample obtained from a pregnant female; (b) calculating a risk score based upon measured levels of the labeled and/or amplified nucleic acid molecules; and (c) administering one or more therapies to the pregnant female based on the risk score, to treat or prevent placental dysfunction. In some embodiments, the method further comprises a step of determining gestational age at blood draw (GABD). In some embodiments, determining GABD is performed before the detecting step.

In some embodiments, this disclosure provides a method of treating or preventing placental dysfunction in a pregnant female, wherein the nucleic acid biomarkers are normalized. For example, in some embodiments, normalization comprises a reversal of the isolated nucleic acid biomarkers.

In some embodiments, this disclosure provides a method of treating or preventing placental dysfunction in a pregnant female, wherein the reversal comprises an upregulated biomarker and a downregulated biomarker. In some embodiments, the risk score is calculated based on the reversal of data values.

In some embodiments, this disclosure provides a method of treating or preventing placental dysfunction in a pregnant female, wherein GABD is between 119-153 days, 154-196 days, and/or 119-202 days.

In some embodiments, this disclosure provides a method of treating or preventing placental dysfunction in a pregnant female, wherein the method determines a pregnant female's risk of developing placental dysfunction across the full window of gestation. In some embodiments, the full gestation window of gestation is between 119-202 days of gestation.

In some embodiments, this disclosure provides a method of treating or preventing placental dysfunction in a pregnant female, wherein the method determines a pregnant female's risk of developing placental dysfunction early in the pregnancy. In some embodiments, early pregnancy is between 119-153 days of gestation.

In some embodiments, this disclosure provides a method of treating or preventing placental dysfunction in a pregnant female, wherein the method determines a pregnant female's risk of developing placental dysfunction later in the pregnancy. In some embodiments, late pregnancy is between 154-196 days of gestation.

In some embodiments, this disclosure provides a method of treating or preventing placental dysfunction in a pregnant female, wherein the placental dysfunction is preeclampsia.

In some embodiments, this disclosure provides a method of treating or preventing placental dysfunction in a pregnant female, wherein calculating the risk score is performed using a binary decision tree analysis.

In some embodiments, this disclosure provides a method of treating or preventing placental dysfunction in a pregnant female, wherein the binary decision tree analysis comprises comparing reversal values of two or more biomarker pairs to two or more predetermined cutoff values and assigning the risk score based on the results of said comparison.

In some embodiments, this disclosure provides a method of treating or preventing placental dysfunction in a pregnant female, wherein calculating the risk score comprises comparing a reversal value of a first biomarker pair from the panel to a first predetermined cutoff, wherein, when the reversal score is greater than or less than the first predetermined cutoff, the risk score is calculated from a comparison of a reversal value of a second biomarker pair from the panel to a second predetermined cutoff value.

In some embodiments, this disclosure provides a method of treating or preventing placental dysfunction in a pregnant female, wherein calculating the risk score further comprises, when the reversal score of the second biomarker pair is greater than or less than the second predetermined cutoff, the risk score is calculated from a comparison of a reversal value of a third biomarker pair from the panel to a third predetermined cutoff value.

In some embodiments, this disclosure provides a method of treating or preventing placental dysfunction in a pregnant female, wherein the risk score is assigned a value based on whether the reversal value of the third biomarker pair is greater than or less than the third predetermined cutoff value.

In some embodiments, this disclosure provides a method of treating or preventing placental dysfunction in a pregnant female, wherein calculating the risk score comprises comparing a reversal value of a first biomarker pair to a first predetermined cutoff and, when the reversal score is greater than or less than the first predetermined cutoff, the risk score is assigned a value.

In some embodiments, this disclosure provides a method of treating or preventing placental dysfunction in a pregnant female, wherein calculating the risk score comprises comparing the reversal value of the second biomarker pair to the second predetermined cutoff and, when the reversal score is greater than or less than the second predetermined cutoff, the risk score is assigned a value.

In another aspect, this disclosure provides a method for treating or preventing placental dysfunction in a pregnant female, the method comprising: (a) detecting the panel of isolated nucleic acid biomarkers of any one of panels listed in Table 6 in a biological sample obtained from a pregnant female; (b) providing a risk score based upon measured levels of the labeled and/or amplified nucleic acid molecules; (c) determining that said pregnant female has an increased likelihood of placental dysfunction based at least in part on said risk score exceeding a reference score; and (d) recommending, facilitating, prescribing, or administering one or more therapies to the pregnant female based on the risk score, to treat or prevent placental dysfunction.

In some embodiments, this disclosure provides a method of treating or preventing placental dysfunction in a pregnant female, wherein the nucleic acid biomarkers are normalized. In some embodiments, normalization comprises a reversal of the isolated nucleic acid biomarkers.

In some embodiments, this disclosure provides a method of treating or preventing placental dysfunction in a pregnant female, wherein the reversal comprises an upregulated biomarker and a downregulated biomarker.

In some embodiments, this disclosure provides a method of treating or preventing placental dysfunction in a pregnant female, wherein the risk score is calculated based on the reversal of data values.

In some embodiments, this disclosure provides a method of treating or preventing placental dysfunction in a pregnant female, wherein the method further comprises a step of determining gestational age at blood draw (GABD).

In some embodiments, this disclosure provides a method of treating or preventing placental dysfunction in a pregnant female, wherein determining GABD is performed before the detecting step.

In some embodiments, this disclosure provides a method of treating or preventing placental dysfunction in a pregnant female, wherein GABD is between 119-153 days, 154-196 days, and/or 119-202 days.

In some embodiments, this disclosure provides a method of treating or preventing placental dysfunction in a pregnant female, wherein the method determines a pregnant female's risk of developing placental dysfunction across the full window of gestation.

In some embodiments, this disclosure provides a method of treating or preventing placental dysfunction in a pregnant female, wherein the full gestation window of gestation is between 119-202 days of gestation.

In some embodiments, this disclosure provides a method of treating or preventing placental dysfunction in a pregnant female, wherein the method determines a pregnant female's risk of developing placental dysfunction early in the pregnancy. In some embodiments, early pregnancy is between 119-153 days of gestation.

In some embodiments, this disclosure provides a method of treating or preventing placental dysfunction in a pregnant female, wherein the method determines a pregnant female's risk of developing placental dysfunction later in the pregnancy. In some embodiments, late pregnancy is between 154-196 days of gestation.

In some embodiments, this disclosure provides a method of treating or preventing placental dysfunction in a pregnant female, wherein the placental dysfunction is preeclampsia.

In some embodiments, this disclosure provides a method of treating or preventing placental dysfunction in a pregnant female, wherein calculating the risk score is performed using a binary decision tree analysis.

In some embodiments, this disclosure provides a method of treating or preventing placental dysfunction in a pregnant female, wherein the binary decision tree analysis comprises comparing reversal values of two or more biomarker pairs to two or more predetermined cutoff values and assigning the risk score based on the results of said comparison.

In some embodiments, this disclosure provides a method of treating or preventing placental dysfunction in a pregnant female, wherein calculating the risk score comprises comparing a reversal value of a first biomarker pair from the panel to a first predetermined cutoff, wherein, when the reversal score is greater than or less than the first predetermined cutoff, the risk score is calculated from a comparison of a reversal value of a second biomarker pair from the panel to a second predetermined cutoff value.

In some embodiments, this disclosure provides a method of treating or preventing placental dysfunction in a pregnant female, wherein calculating the risk score further comprises, when the reversal score of the second biomarker pair is greater than or less than the second predetermined cutoff, the risk score is calculated from a comparison of a reversal value of a third biomarker pair from the panel to a third predetermined cutoff value.

In some embodiments, this disclosure provides a method of treating or preventing placental dysfunction in a pregnant female, wherein the risk score is assigned a value based on whether the reversal value of the third biomarker pair is greater than or less than the third predetermined cutoff value.

In some embodiments, calculating the risk score comprises comparing a reversal value of a first biomarker pair to a first predetermined cutoff and, when the reversal score is greater than or less than the first predetermined cutoff, the risk score is assigned a value.

In some embodiments, calculating the risk score comprises comparing the reversal value of the second biomarker pair to the second predetermined cutoff and, when the reversal score is greater than or less than the second predetermined cutoff, the risk score is assigned a value.

In some embodiments, the invention provides a method of treating or preventing placental dysfunction wherein at least one of the biomarkers is selected from the group consisting of hsa-miR-517a-3p-hsa-miR-517b-3p, hsa-miR-516b-5p, hsa-miR-760, hsa-miR-6852-5p, and hsa-miR-1323.

In some embodiments, the invention provides a method of treating or preventing placental dysfunction wherein at least one of the biomarkers of hsa-miR-30c-5p, hsa-miR-485-5p, hsa-miR-331-3p, hsa-miR-517a-3p-hsa-miR-517b-3p, hsa-miR-516b-5p, hsa-miR-155-5p, hsa-miR-6747-3p, hsa-miR-181d-5p, and hsa-miR-4446-3p.

In some embodiments, the invention provides a method of treating or preventing placental dysfunction wherein at least one of the biomarkers hsa-miR-30c-5p, hsa-miR-6842-3p, hsa-miR-485-5p, hsa-miR-331-3p, hsa-miR-760, hsa-miR-6747-3p, hsa-miR-181d-5p, and hsa-miR-4446-3p.

In some embodiments, the isolated nucleic acid biomarkers are normalized. In further embodiments, normalization comprises a reversal. In some embodiments, the reversal comprises an upregulated biomarker and a downregulated biomarker. In some embodiments, the reversal comprises a numerator and a denominator. In further embodiments, the downregulated biomarker is hsa-miR-485-5p. In other embodiments, the denominator is hsa-miR-485-5p.

In some embodiments, the invention provides a method wherein the risk score is calculated based on the reversal of data values.

In some embodiments, the method further comprises a step of determining gestational age at blood draw (GABD). In further embodiments, the method comprises determining GABD before detecting the biomarker. In some embodiments, GABD is between 119-153 days, 154-196 days and/or 119-202 days.

In some embodiments, the invention provides a method of treating or preventing placental dysfunction across the full window of gestation. In some embodiments, the full gestation window of gestation is between 119-202 days of gestation.

In some embodiments, the invention provides a method of treating or preventing placental dysfunction early in the pregnancy. In some embodiments, early pregnancy is between 119-153 days of gestation.

In some embodiments, the invention provides a method of treating or preventing placental dysfunction later in the pregnancy. In some embodiments, late pregnancy is between 154-196 days of gestation.

Therapies that can be administered to treat or prevent placental dysfunction include bedrest, steroid injection, low-dose aspirin, statins, metformin, and care management/elevated care inclusive of serial blood pressure and intensive symptomatic monitoring, and lifestyle and behavioral modification. A person skilled in the art can readily contemplate these and/or additional therapies that can be used to treat or prevent placental dysfunction.

### Examples

### Example 1: Identification of Extracellular Micro RNA Biomarkers for

### Identification of Pregnancies at Risk for Placental Dysfunction

This example shows identification of extracellular miRNA biomarkers for prediction of preeclampsia: placental dysfunction affecting maternal blood pressure and renal, liver and central nervous system function.

### Study Design

Unblinded samples were used for this discovery study. Additional blinded samples are reserved for future re-verification or validation.

Maternal serum samples were collected from high-risk and average-risk pregnant women between 17-28 weeks, for which pregnancy outcomes are known. The sample set comprised 212 samples, including 73 preeclampsia and 139 normal. GABD was at a minimum of 120 days to a maximum of 201 days with a mean of 163.6 days.

TABLE 1
Pre-specified subject classifications

| | Range | Range of | | | | | |
|---|---|---|---|---|---|---|---|
| | of GA at | GA at | Criterion | Criterion | Criterion | Criterion | Criterion |
| Group | draw | birth | 1 | 2 | 3 | 4 | 5 |
| UCSD | 19-27 | 25-41 | New-onset | Chronic | Chronic | New-onset or | New-onset |
| cases | weeks | weeks | hypertension | hypertensi | proteinuria | chronic | or chronic |
| | | | and new- | on and | and new- | hypertension | proteinuria |
| | | | onset | new-onset | onset | and new- | and new- |
| | | | proteinuria | proteinuria | hypertensi | onset severe | onset severe |
| | | | OR | OR | on OR | feature | feature |
| | | | | | | (elevated | (elevated |
| | | | | | | LFTs, | LFTs, |
| | | | | | | elevated Cr, | elevated Cr, |
| | | | | | | low platelets, | low |
| | | | | | | IUGR) OR | platelets, |
| | | | | | | | IUGR) |
| Sera | 17 1/7-28 | 25 5/7-40 | New-onset | Superimposed | | Proteinuria | |
| PAPR | 5/7 weeks | 5/7 | hypertension | preeclamps | | and new- | |
| cases | | | with or | ia with or | | onset severe | |
| | | | without | without | | feature | |
| | | | severe | severe | | (elevated | |
| | | | features OR | features, | | LFTs, | |
| | | | | OR | | elevated Cr, | |
| | | | | | | low platelets, | |
| | | | | | | IUGR) | |
| UCSD | 19-27 | 25-41 | No | | | | |
| controls | weeks | weeks | hypertensive | | | | |
| | | | disease | | | | |
| Sera | 17 1/7-28 | 37 0/7-41 | | | | | |
| PAPR | 5/7 weeks | 4/7 weeks | | | | | |
| controls | | | | | | | |

TABLE 2
Sample, maternal, and pregnancy conditions and identifiers

| Field | Description |
|---|---|
| Batch | Small RNAseq analysis batch |
| SampleName | Sample Name |
| GASampleCollection | Gestational Age at Sample Collection |
| GADelivery | Gestational Age at Delivery |
| BMI | First recorded maternal BMI during |
| | pregnancy episode |
| MaternalAge | Maternal Age at Delivery |
| Race | Maternal Race (self-reported) |
| Ethnicity | Maternal Ethnicity (self-reported) |
| Birthweight | Birthweight |
| Gender | Fetal sex |
| BW % | Birthweight Percentile |
| | (Calculated using Hadlock and Fenton) |
| Diagnosis | Detailed Placental Dysfunction Diagnosis |
| Diagnosis_simple | Simple Placental Dysfunction Diagnosis |
| | (Normal, Pree) |
| Diagnosis_mild_severe | Intermediate Placental Dysfunction Diagnosis |
| | (Normal, Pree_Mild, Pree_Severe) |
| Diagnosis_Diabetes | Diabetes Diagnosis |
| | (No Diabetes, GDM, T2DM, T1DM) |
| IUGR | Birthweight <10^{th} percentile (YES, NO) |

### Lab Analysis

Total extracellular RNA (exRNA) from each sample was purified and subjected to small RNA sequencing.

Total extracellular RNA was purified from 500 μL serum using the miRNeasy micro kit (Qiagen), followed by the RNA Clean and Concentrator Kit (Zymo), with a final elution volume of 12 μL. 1.2 μL of the resulting exRNA was used to prepare the Small RNAseq libraries using the NEBNext Small RNA Sequencing Library Preparation Kit, using the manufacturer's instruction except for the following: 1. Adapters were diluted 1:6; 2. The reactions were run at ⅕^{th} volume using a mosquito HTS liquid handler. Up to 384 libraries were prepared in a given batch (some of the libraries for this project were prepared in the same batches as other projects), and libraries were multiplexed using the available 48 NEB Small RNAseq indices. Up to 48 samples were combined per pool, and each pool was size selected using a Pippin Prep (either 177-180 bp, or 125-160 bp). Samples were sequenced on a HiSeq 4000. Each pool of up to 48 samples was loaded onto its own lane, generating at least 350 million single-end 75 bp reads.

### Data Analysis

Data preprocessing, including adapter trimming and mapping to miRBase (ref to miRbase version), was performed using the ExceRpt pipeline on the Genboree Workbench (which can be accessed at exRNA.org), to yield Raw Count data.

Further filtering removed individual miRNAs with >70% missing values. Batch normalization was carried out using Variance Stabilizing Transformation and Bias Reduction. The PEER package (Sanger Institute) was run to further to reduce batch effect and amplify preeclampsia signal. Replicate data was then collapsed to single values. AUCs were generated with the pROC package, using the Delong and bootstrap methods to establish the confidence intervals (CIs). Analysis was performed using R 3.6.3 (Robin et al., BMC bioinformatics 12:77 (2011)).

Univariate miRNA models were fit to the entire GABD dataset range with an interaction term with GABD to allow for GABD dependence of performance. Univariate models and bivariate reversals (ratios of miRNAs) were selected by ranking performance.

Lower 25% DeLong's CI and AUC for prediction of Preeclampsia was determined.

Pearson correlation of score with diagnosis of Preeclampsia (1) or not (0) was determined.

T-test p-value significance in score between cases and controls was determined.

Numeric performance data were converted to ranks, inverted and summed to produce a final ranking.

Top ranking reversals may be:
a) Formed into tree-like or additive (lasso) panel hypotheses of robust performance across multiple weeks.
b) Formed into combined protein/RNA hypotheses to the extent that proteomic data are available in the same samples.
c) Re-verified or validated as univariate, bivariate and panel hypotheses in a future small RNA sequencing data set, for example comprising blinded UCSD samples.

TABLE 3
Top-ranked single microRNAs predicting
preeclampsia as interaction terms with GABD

| | Lower | | | | |
|---|---|---|---|---|---|
| | 25% CI | | Correlation | T-test | Final |
| microRNA | of AUC | AUC | (R\hat{ }2) | p-value | ranking |
| hsa-miR-516b-5p | 0.573 | 0.649 | 0.228 | 2.67E−03 | 1 |
| ha-miR-30c-5p | 0.532 | 0.616 | 0.218 | 4.72E−03 | 2 |
| hsa-miR-1-3p | 0.550 | 0.629 | 0.155 | 2.03E−02 | 3 |
| hsa-miR-331-3p | 0.537 | 0.617 | 0.201 | 5.79E−03 | 4 |
| hsa-miR-485-5p | 0.525 | 0.606 | 0.207 | 5.13E−03 | 5 |
| hsa-miR-181d-5p | 0.550 | 0.628 | 0.170 | 1.54E−02 | 6 |
| hsa-miR-23a-3p | 0.531 | 0.612 | 0.197 | 7.33E−03 | 7 |
| hsa-miR-378d | 0.521 | 0.601 | 0.201 | 6.72E−03 | 8 |
| hsa-miR-155-5p | 0.531 | 0.610 | 0.176 | 1.28E−02 | 9 |
| hsa-miR-99b-3p | 0.527 | 0.608 | 0.173 | 1.37E−02 | 10 |
| hsa-miR-1249-3p | 0.521 | 0.602 | 0.179 | 1.14E−02 | 11 |
| hsa-miR-142-3p | 0.507 | 0.587 | 0.189 | 7.71E−03 | 12 |
| hsa-miR-4433b-5p | 0.525 | 0.604 | 0.180 | 1.44E−02 | 13 |
| hsa-miR-1273h-3p | 0.513 | 0.593 | 0.181 | 9.15E−03 | 14 |
| hsa-miR-18a-3p | 0.528 | 0.606 | 0.143 | 2.74E−02 | 15 |
| hsa-miR-4746-5p | 0.527 | 0.607 | 0.142 | 3.42E−02 | 16 |
| hsa-miR-27a-5p | 0.527 | 0.605 | 0.147 | 2.45E−02 | 17 |
| hsa-miR-654-3p | 0.512 | 0.594 | 0.175 | 1.17E−02 | 18 |
| hsa-miR-7-5p | 0.502 | 0.584 | 0.182 | 1.56E−02 | 19 |
| hsa-miR-941 | 0.520 | 0.599 | 0.152 | 2.09E−02 | 20 |

TABLE 4
Top ranked pairs of microRNAs predicting
preeclampsia as a ratio in an interaction term with GABD

| | Lower | | | | |
|---|---|---|---|---|---|
| | 25% CI | | Correlation | T-test | Final |
| microRNA pair | of AUC | AUC | (R\hat{ }2) | p-value | ranking |
| hsa-miR-140-3p/ | 0.611 | 0.684 | 0.319 | 8.26E−06 | 1 |
| hsa-miR-516b-5p | | | | | |
| hsa-miR-155-5p/ | 0.610 | 0.685 | 0.201 | 4.23E−03 | 2 |
| hsa-miR-625-3p | | | | | |
| hsa-miR-30c-5p/ | 0.596 | 0.674 | 0.337 | 2.12E−05 | 3 |
| hsa-miR-27b-3p | | | | | |
| hsa-miR-25-5p/ | 0.603 | 0.679 | 0.326 | 2.65E−05 | 4 |
| hsa-miR-516b-5p | | | | | |
| hsa-miR-484/ | 0.599 | 0.673 | 0.310 | 2.05E−05 | 5 |
| hsa-miR-516b-5p | | | | | |
| hsa-miR-516b-5p/ | 0.605 | 0.678 | 0.289 | 1.17E−04 | 6 |
| hsa-let-7b-5p | | | | | |
| hsa-miR-30c-5p/ | 0.590 | 0.667 | 0.324 | 2.92E−05 | 7 |
| hsa-miR-7-5p | | | | | |
| hsa-miR-516b-5p/ | 0.605 | 0.679 | 0.282 | 2.02E−04 | 8 |
| hsa-miR-486-5p | | | | | |
| hsa-miR-516b-5p/ | 0.601 | 0.675 | 0.305 | 6.54E−05 | 9 |
| hsa-miR-150-5p | | | | | |
| hsa-miR-485-5p/ | 0.594 | 0.674 | 0.314 | 4.10E−05 | 10 |
| hsa-miR-516b-5p | | | | | |
| hsa-miR-7-5p/ | 0.595 | 0.672 | 0.317 | 1.01E−04 | 11 |
| hsa-miR-485-5p | | | | | |
| hsa-miR-516b-5p/ | 0.603 | 0.676 | 0.282 | 1.88E−04 | 12 |
| hsa-let-7g-5p | | | | | |
| hsa-miR-30c-5p/ | 0.591 | 0.668 | 0.316 | 8.97E−05 | 13 |
| hsa-miR-16-5p | | | | | |
| hsa-miR-516b-5p/ | 0.603 | 0.676 | 0.268 | 3.63E−04 | 14 |
| hsa-miR-423-5p | | | | | |
| hsa-miR-30c-5p/ | 0.579 | 0.658 | 0.310 | 7.25E−05 | 15 |
| hsa-miR-30a-5p | | | | | |
| hsa-miR-516b-5p/ | 0.601 | 0.675 | 0.275 | 3.65E−04 | 16 |
| hsa-miR-92a-3p | | | | | |
| hsa-miR-186-5p/ | 0.594 | 0.669 | 0.298 | 8.50E−05 | 17 |
| hsa-miR-516b-5p | | | | | |
| hsa-miR-155-5p/ | 0.599 | 0.676 | 0.235 | 1.40E−03 | 18 |
| hsa-miR-27b-3p | | | | | |
| hsa-miR-30e-5p/ | 0.600 | 0.675 | 0.263 | 3.05E−04 | 19 |
| hsa-miR-516b-5p | | | | | |
| hsa-miR-516b-5p/ | 0.600 | 0.674 | 0.278 | 3.96E−04 | 20 |
| hsa-let-7d-5p | | | | | |
| hsa-miR-516b-5p/ | 0.599 | 0.674 | 0.281 | 2.53E−04 | 21 |
| hsa-miR-103a-3p | | | | | |
| hsa-miR-516b-5p/ | 0.600 | 0.673 | 0.275 | 3.28E−04 | 22 |
| hsa-miR-126-3p | | | | | |
| hsa-miR-99b-3p/ | 0.593 | 0.670 | 0.288 | 1.19E−04 | 23 |
| hsa-miR-486-3p | | | | | |
| hsa-miR-6741-5p/ | 0.579 | 0.657 | 0.300 | 1.29E−04 | 24 |
| hsa-miR-760 | | | | | |
| hsa-miR-20a-5p/ | 0.588 | 0.666 | 0.299 | 2.19E−04 | 25 |
| hsa-miR-485-5p | | | | | |
| hsa-miR-23a-3p/ | 0.590 | 0.668 | 0.294 | 1.54E−04 | 26 |
| hsa-miR-27b-3p | | | | | |
| hsa-miR-516b-5p/ | 0.600 | 0.673 | 0.259 | 6.97E−04 | 27 |
| hsa-miR-151a-3p | | | | | |
| hsa-miR-30a-5p/ | 0.577 | 0.655 | 0.305 | 3.05E−04 | 28 |
| hsa-miR-485-5p | | | | | |
| hsa-miR-516b-5p/ | 0.598 | 0.673 | 0.280 | 3.33E−04 | 29 |
| hsa-let-7a-5p | | | | | |
| hsa-miR-30c-5p/ | 0.576 | 0.655 | 0.298 | 1.57E−04 | 30 |
| hsa-miR-27a-3p | | | | | |
| hsa-miR-485-5p/ | 0.575 | 0.654 | 0.296 | 1.32E−04 | 31 |
| hsa-miR-7-5p | | | | | |
| hsa-miR-516b-5p/ | 0.599 | 0.673 | 0.252 | 8.72E−04 | 32 |
| hsa-miR-222-3p | | | | | |
| hsa-miR-23a-3p/ | 0.576 | 0.655 | 0.285 | 1.17E−04 | 33 |
| hsa-miR-30a-5p | | | | | |
| hsa-miR-433-3p/ | 0.597 | 0.671 | 0.268 | 4.74E−04 | 34 |
| hsa-miR-345-5p | | | | | |
| hsa-miR-516b-5p/ | 0.594 | 0.669 | 0.280 | 3.32E−04 | 35 |
| hsa-let-7f-5p | | | | | |
| hsa-miR-485-5p/ | 0.583 | 0.662 | 0.286 | 1.75E−04 | 36 |
| hsa-miR-183-5p | | | | | |
| hsa-miR-516b-5p/ | 0.597 | 0.672 | 0.252 | 9.93E−04 | 37 |
| hsa-miR-185-5p | | | | | |
| hsa-let-7i-5p/ | 0.577 | 0.656 | 0.298 | 4.43E−04 | 38 |
| hsa-miR-485-5p | | | | | |
| hsa-miR-4433b-5p/ | 0.585 | 0.661 | 0.280 | 1.38E−04 | 39 |
| hsa-miR-516b-5p | | | | | |
| hsa-miR-516b-5p/ | 0.595 | 0.670 | 0.262 | 5.94E−04 | 40 |
| hsa-miR-191-5p | | | | | |
| hsa-miR-516b-5p/ | 0.596 | 0.671 | 0.249 | 9.89E−04 | 41 |
| hsa-miR-3615 | | | | | |
| hsa-miR-100-5p/ | 0.576 | 0.652 | 0.271 | 1.28E−04 | 42 |
| hsa-miR-516b-5p | | | | | |
| hsa-miR-516b-5p/ | 0.595 | 0.669 | 0.267 | 6.35E−04 | 43 |
| hsa-miR-10b-5p | | | | | |
| hsa-miR-485-5p/ | 0.573 | 0.653 | 0.287 | 1.91E−04 | 44 |
| hsa-miR-27a-3p | | | | | |
| hsa-miR-99a-5p/ | 0.568 | 0.648 | 0.298 | 4.88E−04 | 45 |
| hsa-miR-485-5p | | | | | |
| hsa-miR-99b-3p/ | 0.583 | 0.659 | 0.281 | 2.14E−04 | 46 |
| hsa-miR-6852-5p | | | | | |
| hsa-miR-363-3p/ | 0.571 | 0.651 | 0.294 | 3.88E−04 | 47 |
| hsa-miR-485-5p | | | | | |
| hsa-miR-516b-5p/ | 0.587 | 0.662 | 0.276 | 2.82E−04 | 48 |
| hsa-miR-484 | | | | | |
| hsa-miR-516b-5p/ | 0.593 | 0.666 | 0.269 | 3.95E−04 | 49 |
| hsa-let-7i-5p | | | | | |
| hsa-miR-23a-3p/ | 0.585 | 0.663 | 0.277 | 2.57E−04 | 50 |
| hsa-miR-181a-5p | | | | | |
| hsa-miR-7-5p/ | 0.583 | 0.658 | 0.269 | 1.90E−04 | 51 |
| hsa-miR-516b-5p | | | | | |
| hsa-miR-516b-5p/ | 0.590 | 0.665 | 0.270 | 3.72E−04 | 52 |
| hsa-miR-328-3p | | | | | |
| hsa-miR-181a-5p/ | 0.577 | 0.655 | 0.292 | 5.70E−04 | 53 |
| hsa-miR-485-5p | | | | | |
| hsa-miR-155-5p/ | 0.593 | 0.670 | 0.229 | 1.16E−03 | 54 |
| hsa-miR-30a-5p | | | | | |
| hsa-miR-155-5p/ | 0.593 | 0.670 | 0.202 | 4.71E−03 | 55 |
| hsa-let-7i-5p | | | | | |
| hsa-miR-98-5p/ | 0.572 | 0.650 | 0.289 | 3.58E−04 | 56 |
| hsa-miR-485-5p | | | | | |
| hsa-miR-485-5p/ | 0.579 | 0.656 | 0.276 | 2.37E−04 | 57 |
| hsa-miR-127-3p | | | | | |
| hsa-miR-320b/ | 0.565 | 0.644 | 0.294 | 6.13E−04 | 58 |
| hsa-miR-485-5p | | | | | |
| hsa-miR-516b-5p/ | 0.590 | 0.664 | 0.267 | 4.74E−04 | 59 |
| hsa-miR-25-3p | | | | | |
| hsa-miR-516b-5p/ | 0.593 | 0.668 | 0.242 | 1.44E−03 | 60 |
| hsa-miR-148a-3p | | | | | |
| hsa-miR-516b-5p/ | 0.591 | 0.665 | 0.263 | 6.09E−04 | 61 |
| hsa-miR-128-3p | | | | | |
| hsa-miR-516b-5p/ | 0.590 | 0.665 | 0.263 | 5.60E−04 | 62 |
| hsa-miR-320a | | | | | |
| hsa-miR-516b-5p/ | 0.592 | 0.666 | 0.252 | 8.68E−04 | 63 |
| hsa-miR-744-5p | | | | | |
| hsa-miR-30c-5p/ | 0.559 | 0.639 | 0.292 | 4.21E−04 | 64 |
| hsa-miR-186-5p | | | | | |
| hsa-miR-516b-5p/ | 0.588 | 0.664 | 0.259 | 5.52E−04 | 65 |
| hsa-miR-629-5p | | | | | |
| hsa-miR-23a-3p/ | 0.576 | 0.654 | 0.277 | 3.02E−04 | 66 |
| hsa-miR-93-5p | | | | | |
| hsa-miR-127-3p/ | 0.572 | 0.650 | 0.289 | 6.46E−04 | 67 |
| hsa-miR-485-5p | | | | | |
| hsa-miR-155-5p/ | 0.591 | 0.668 | 0.204 | 3.91E−03 | 68 |
| hsa-miR-451a | | | | | |
| hsa-miR-30c-5p/ | 0.545 | 0.623 | 0.278 | 1.94E−04 | 69 |
| hsa-miR-26b-5p | | | | | |
| hsa-miR-4732-3p/ | 0.580 | 0.658 | 0.249 | 3.20E−04 | 70 |
| hsa-miR-516b-5p | | | | | |
| hsa-miR-7-5p/ | 0.589 | 0.665 | 0.257 | 1.05E−03 | 71 |
| hsa-miR-941 | | | | | |
| hsa-miR-451a/ | 0.570 | 0.650 | 0.288 | 6.74E−04 | 72 |
| hsa-miR-485-5p | | | | | |
| hsa-miR-374b-5p/ | 0.547 | 0.628 | 0.292 | 5.74E−04 | 73 |
| hsa-miR-885-3p | | | | | |
| hsa-miR-516b-5p/ | 0.587 | 0.662 | 0.259 | 5.89E−04 | 74 |
| hsa-miR-1307-3p | | | | | |
| hsa-miR-516b-5p/ | 0.588 | 0.662 | 0.256 | 8.34E−04 | 75 |
| hsa-miR-30d-5p | | | | | |
| hsa-miR-3605-5p/ | 0.580 | 0.656 | 0.264 | 3.41E−04 | 76 |
| hsa-miR-425-3p | | | | | |
| hsa-miR-501-3p/ | 0.571 | 0.647 | 0.260 | 2.38E−04 | 77 |
| hsa-miR-516b-5p | | | | | |
| hsa-miR-125b-5p/ | 0.573 | 0.652 | 0.286 | 6.26E−04 | 78 |
| hsa-miR-485-5p | | | | | |
| hsa-miR-26b-5p/ | 0.578 | 0.654 | 0.279 | 4.64E−04 | 79 |
| hsa-miR-485-5p | | | | | |
| hsa-miR-30c-5p/ | 0.565 | 0.645 | 0.281 | 3.90E−04 | 80 |
| hsa-miR-99a-5p | | | | | |
| hsa-miR-516b-5p/ | 0.589 | 0.664 | 0.239 | 1.42E−03 | 81 |
| hsa-miR-127-3p | | | | | |
| hsa-miR-501-3p/ | 0.564 | 0.643 | 0.287 | 5.90E−04 | 82 |
| hsa-miR-485-5p | | | | | |
| hsa-miR-30c-5p/ | 0.569 | 0.650 | 0.282 | 5.45E−04 | 83 |
| hsa-miR-451a | | | | | |
| hsa-miR-21-5p/ | 0.562 | 0.641 | 0.288 | 7.62E−04 | 84 |
| hsa-miR-485-5p | | | | | |
| hsa-miR-516b-5p/ | 0.586 | 0.661 | 0.259 | 7.42E−04 | 85 |
| hsa-miR-423-3p | | | | | |
| hsa-miR-516b-5p/ | 0.587 | 0.662 | 0.247 | 9.06E−04 | 86 |
| hsa-miR-146b-5p | | | | | |
| hsa-miR-330-5p/ | 0.585 | 0.663 | 0.261 | 1.51E−03 | 87 |
| hsa-miR-92b-5p | | | | | |
| hsa-miR-4443/ | 0.545 | 0.627 | 0.290 | 8.81E−04 | 88 |
| hsa-miR-485-5p | | | | | |
| hsa-miR-30c-5p/ | 0.559 | 0.642 | 0.285 | 5.10E−04 | 89 |
| hsa-let-7i-5p | | | | | |
| hsa-miR-516b-5p/ | 0.586 | 0.662 | 0.250 | 7.93E−04 | 90 |
| hsa-miR-425-5p | | | | | |
| hsa-miR-516b-5p/ | 0.587 | 0.661 | 0.256 | 8.04E−04 | 91 |
| hsa-miR-24-3p | | | | | |
| hsa-miR-146b-5p/ | 0.572 | 0.650 | 0.283 | 8.97E−04 | 92 |
| hsa-miR-485-5p | | | | | |
| hsa-miR-760/ | 0.565 | 0.642 | 0.261 | 3.01E−04 | 93 |
| hsa-miR-378g | | | | | |
| hsa-miR-3615/ | 0.576 | 0.653 | 0.281 | 9.17E−04 | 94 |
| hsa-miR-485-5p | | | | | |
| hsa-miR-140-3p/ | 0.564 | 0.644 | 0.281 | 5.23E−04 | 95 |
| hsa-miR-485-5p | | | | | |
| hsa-miR-30c-5p/ | 0.566 | 0.647 | 0.280 | 4.74E−04 | 96 |
| hsa-miR-125a-5p | | | | | |
| hsa-miR-378c/ | 0.571 | 0.647 | 0.259 | 3.23E−04 | 97 |
| hsa-miR-148a-5p | | | | | |
| hsa-let-7b-5p/ | 0.577 | 0.655 | 0.280 | 9.35E−04 | 98 |
| hsa-miR-485-5p | | | | | |
| hsa-miR-516b-5p/ | 0.583 | 0.657 | 0.258 | 6.27E−04 | 99 |
| hsa-miR-146a-5p | | | | | |
| hsa-miR-516b-5p/ | 0.583 | 0.658 | 0.259 | 6.90E−04 | 100 |
| hsa-miR-451a | | | | | |

### Example 2: Identification of Extracellular Micro RNA Biomarkers Across Three

### Gestational Windows for Identification of Pregnancies at Risk for Placental Dysfunction

This example shows identification of extracellular miRNA biomarkers across three gestational windows for prediction of preeclampsia: placental dysfunction affecting maternal blood pressure and renal, liver and central nervous system function.

### Study Design and Lab Analysis

Samples were collected and analyzed as described above in Example 1.

### Data Analysis

miRNAs were evaluated across three windows of gestational age at blood draw (GABD) including early (119-153), late (154-196) and full (119-202) days of gestation. A variation of a stacking algorithm was used, in which four modeling approaches were combined with a brute force approach to rank each miRNA in the dataset within each GABD window by an inverse rank sum metric. The four algorithms were MRPC using MRPC ≥2.2.1, LASSO using glmnet ≥4.0, conditional inference trees using party ≥1.3 and gradient boosting using xgboost ≥1.2.0.1. To control for analytic variability of reversals (ratios of log counts) of miRNAs, instead of single miRNAs, were identified as predictors in candidate models for validation. To assess performance of miRNAs within reversals, a fifth, brute-force ranking based on significance of enrichment of individual miRNAs amongst the top performing reversals was added. Top reversals per GABD window were determined by first ranking all reversals by inverse rank sum, then incrementally building a matrix of reversal scores in order of ranking and selecting as a cutpoint the number of reversals in the matrix at which a plateau was reached for significance of association of the first principal component in PCA with the outcome. Each of the different learning methods ranked the miRNAs from most important to least important and the final score was determined by taking a weighted inverse rank sum across all the different models plus the brute force approach. This approach generated the list of miRNAs in Table 5.

To further characterize miRNAs in Table 5, two additional analyses were applied. First, it was tested whether the difference in the logarithm of miRNA levels between cases and controls was significant by the Wilcoxon test in processed miRNA assay data. Second, it was tested whether the direction of median shift seen in the processed data could be reproduced in noisy data in a future assay as represented by the raw RNAseq data before processing.

TABLE 5
Top ranked microRNAs predicting preeclampsia in an interaction term
with GABD windows.

| | | | | Cases | Cases |
|---|---|---|---|---|---|
| | | | Cases | relative | relative |
| | | | relative to | to | to |
| | | Window(s), | controls, | controls, | controls, |
| | | with batch- | full | early | late |
| | Significant | independent | window | window | window |
| | in | median | (GABD | (GABD | (GABD |
| microRNA | Windows | shift | 119-202) | 119-153) | 154-196) |
| hsa-miR-30c-5p | full, late | early | lower | lower | lower |
| hsa-miR-6842-3p | full | early, late | lower | lower | lower |
| hsa-miR-485-5p | full, late | full, late | lower | lower | lower |
| hsa-miR-331-3p | full, late | full, early, | lower | lower | lower |
| | | late | | | |
| hsa-miR-517a-3p- | early, late | early, late | lower | lower | higher |
| hsa-miR-517b-3p | | | | | |
| hsa-miR-516b-5p | early, late | early, late | higher | lower | higher |
| hsa-miR-760 | full, early | full, late | lower | lower | lower |
| hsa-miR-6852-5p | early | full | higher | higher | lower |
| hsa-miR-1323 | early | full, early, | lower | lower | higher |
| | | late | | | |
| hsa-miR-155-5p | late | full, early, | lower | higher | lower |
| | | late | | | |
| hsa-miR-6747-3p | full, late | full, late | lower | higher | lower |
| hsa-miR-181d-5p | late | full, late | lower | higher | lower |
| hsa-miR-4446-3p | full, late | full, early, | lower | higher | lower |
| | | late | | | |

The above analysis identified thirteen miRNAs in Table 5 that can predict preeclampsia in early, late or full gestational age in cases relative to controls. The thirteen miRNAs can predict preeclampsia singly or as reversals of one another.

### Example 3: Identification of Panels of Nucleic Acid Biomarker Pairs

This example describes the identification of panels of miRNA biomarker pairs. The miRNA biomarker pairs can be used for predicting, preventing, or treating of placental dysfunction, e.g., preeclampsia.

Quantities of miRNA were analyzed from RNA-seq data of 269 miRNAs that showed a non-zero detection in at least seventy percent of analyzed samples. Formation of pairwise ratios of log-transformed miRNA abundances multiplied by gestational age at blood draw, hereinafter designated as bivariates or reversals, resulted in 72,092 candidate predictors. The reversals were initially downselected by consensus between multiple independent model types on component microRNAs, followed by identifying those showing significant Pearson correlation to a binary diagnosis of preeclampsia, resulting in a candidate set of 200 reversals.

To allow for non-linear relationships between preeclampsia and microRNA reversal levels, panels were constructed in the form of binary decision tree models from the candidate reversals. Methods for constructing binary decision tree models is described below. Conditional inference trees were used via the party package in R (version ≥1.3-5). Conditional inference trees were preferred over traditional CART models as conditional inference uses statistical significance to create a new branch while CART uses information entropy splits to maximize homogeneity, which shows overfitting and selection bias. For further discussion, see Hothorn T, Hornik K, Zeileis A, 2006, Unbiased Recursive Partitioning: A Conditional Inference Framework, which is incorporated by reference. A depth of 3 branching levels was allowed for each binary decision tree model.

To generate panels of nucleic acid biomarker pairs, 500 models were trained on random 70% subsets of the data and subsequently tested on the remaining 30% of the data, calculating AUCs and their confidence intervals. Trees with the 6 most common primary nodes were selected and with significant performance (i.e. 95% confidence interval of the test set AUC does not include 0.5). The top 6 most common primary nodes were chosen because their trees comprised the majority of the 500 models. As a secondary performance metric, inhomogeneity of terminal nodes was calculated, defined as the proportion of controls in case-dominant terminal nodes plus the proportion of cases in control-dominant terminal nodes. 64 candidate reversals in all were found in the 62 selected models. We calculated summed scores from test set performance and inhomogeneity to rank the models as shown. This approach generated the list of nucleic acid biomarker panels in Table 6.

TABLE 6
Panels of nucleic acid biomarker pairs.

| | | | | | inho- | | |
|---|---|---|---|---|---|---|---|
| | | | lower | Upper | moge- | | rank |
| Model | Panel of Nucleic Acid Biomarker Pairs | AUC | CI | CI | neity | zSum | Sum |
| 1 | hsa-miR-98-5p/hsa-miR-485-5p; hsa-miR-1306- | 0.746 | 0.626 | 0.865 | 0.479 | 3.563 | 2.018 |
| | 3p/hsa-miR-1224-5p; hsa-miR-330-5p/hsa-miR- | | | | | | |
| | 550a-3-5p-hsa-miR-550a-5p; hsa-miR-221-5p/hsa- | | | | | | |
| | miR-197-3p | | | | | | |
| 2 | hsa-miR-7-5p/hsa-miR-485-5p; hsa-miR-1306- | 0.735 | 0.62 | 0.85 | 0.456 | 3.562 | 1.187 |
| | 3p/hsa-miR-664a-5p; hsa-miR-99b-3p/hsa-miR- | | | | | | |
| | 760; hsa-miR-98-5p/hsa-miR-483-3p | | | | | | |
| 3 | hsa-miR-30c-5p/hsa-miR-27b-3p; hsa-miR-433- | 0.749 | 0.619 | 0.879 | 0.501 | 2.929 | 2.350 |
| | 3p/hsa-miR-345-5p; hsa-miR-15b-5p/hsa-miR-197- | | | | | | |
| | 3p; hsa-miR-363-3p/hsa-miR-485-5p | | | | | | |
| 4 | hsa-miR-1306-3p/hsa-miR-664a-5p; hsa-miR-142- | 0.685 | 0.548 | 0.821 | 0.259 | 2.651 | 0.762 |
| | 5p/hsa-miR-485-5p; hsa-miR-15b-5p/hsa-miR-197- | | | | | | |
| | 3p; hsa-miR-7-5p/hsa-miR-485-5p; hsa-miR-433- | | | | | | |
| | 3p/hsa-miR-345-5p | | | | | | |
| 5 | hsa-miR-1306-3p/hsa-miR-664a-5p; hsa-miR- | 0.696 | 0.59 | 0.802 | 0.420 | 2.573 | 0.396 |
| | 378g/hsa-miR-485-5p; hsa-miR-30c-5p/hsa-miR- | | | | | | |
| | 100-5p; hsa-miR-30c-5p/hsa-miR-30a-5p; | | | | | | |
| | hsa-miR-7-5p/hsa-let-7c-5p | | | | | | |
| 6 | hsa-miR-7-5p/hsa-miR-485-5p; hsa-miR-1306- | 0.719 | 0.593 | 0.845 | 0.442 | 2.437 | 0.774 |
| | 3p/hsa-miR-664a-5p; hsa-miR-433-3p/hsa-miR- | | | | | | |
| | 345-5p; hsa-miR-30c-5p/hsa-miR-27b-3p | | | | | | |
| 7 | hsa-miR-7-5p/hsa-miR-485-5p; hsa-miR-1306- | 0.65 | 0.524 | 0.776 | 0.212 | 2.091 | 1.073 |
| | 3p/hsa-miR-664a-5p; hsa-miR-7-5p/hsa-miR-4446- | | | | | | |
| | 3p; hsa-miR-221-5p/hsa-miR-760 | | | | | | |
| 8 | hsa-miR-7-5p/hsa-miR-485-5p; hsa-miR-1306- | 0.684 | 0.576 | 0.792 | 0.408 | 2.052 | 0.286 |
| | 3p/hsa-miR-664a-5p; hsa-miR-501-3p/hsa-miR-484 | | | | | | |
| 9 | hsa-miR-7-5p/hsa-miR-485-5p; hsa-miR-1306- | 0.715 | 0.585 | 0.844 | 0.444 | 2.016 | 0.589 |
| | 3p/hsa-miR-664a-5p; hsa-miR-99b-3p/hsa-miR- | | | | | | |
| | 760; hsa-miR-221-5p/hsa-miR-760 | | | | | | |
| 10 | hsa-miR-7-5p/hsa-miR-485-5p; hsa-miR-1306- | 0.677 | 0.565 | 0.79 | 0.382 | 1.860 | 0.223 |
| | 3p/hsa-miR-664a-5p; hsa-miR-99b-3p/hsa-miR- | | | | | | |
| | 379-5p; hsa-miR-221-5p/hsa-miR-197-3p | | | | | | |
| 11 | hsa-miR-7-5p/hsa-miR-485-5p; hsa-miR-1-3p/hsa- | 0.686 | 0.566 | 0.806 | 0.396 | 1.718 | 0.293 |
| | miR-345-5p; hsa-miR-99b-3p/hsa-miR-150-3p; | | | | | | |
| | hsa-miR-30c-5p/hsa-miR-146a-5p | | | | | | |
| 12 | hsa-miR-7-5p/hsa-miR-485-5p; hsa-miR-1306- | 0.664 | 0.542 | 0.786 | 0.309 | 1.696 | 0.268 |
| | 3p/hsa-miR-664a-5p; hsa-miR-628-3p/hsa-miR- | | | | | | |
| | 19b-3p | | | | | | |
| 13 | hsa-miR-7-5p/hsa-miR-485-5p; hsa-miR-1306- | 0.68 | 0.546 | 0.813 | 0.362 | 1.193 | 0.236 |
| | 3p/hsa-miR-664a-5p; hsa-miR-30c-5p/hsa-miR-7- | | | | | | |
| | 5p; hsa-miR-221-5p/hsa-miR-760 | | | | | | |
| 14 | hsa-miR-7-5p/hsa-miR-485-5p; hsa-miR-7-5p/hsa- | 0.682 | 0.554 | 0.811 | 0.402 | 1.060 | 0.214 |
| | miR-4446-3p; hsa-miR-330-5p/hsa-miR-550a-3-5p- | | | | | | |
| | hsa-miR-550a-5p; hsa-let-7i-5p/hsa-miR-191-5p; | | | | | | |
| | hsa-miR-760/hsa-miR-345-5p; hsa-miR-125a- | | | | | | |
| | 3p/hsa-miR-379-5p | | | | | | |
| 15 | hsa-miR-98-5p/hsa-miR-485-5p; hsa-miR-1306- | 0.671 | 0.558 | 0.784 | 0.427 | 0.922 | 0.161 |
| | 3p/hsa-miR-664a-5p; hsa-miR-99b-3p/hsa-miR-22- | | | | | | |
| | 3p; hsa-miR-221-5p/hsa-miR-760 | | | | | | |
| 16 | hsa-miR-7-5p/hsa-miR-485-5p; hsa-miR-433- | 0.649 | 0.516 | 0.782 | 0.282 | 0.780 | 0.399 |
| | 3p/hsa-miR-345-5p; hsa-miR-15b-5p/hsa-miR-197- | | | | | | |
| | 3p; hsa-miR-330-5p/hsa-miR-223-3p | | | | | | |
| 17 | hsa-miR-1306-3p/hsa-miR-664a-5p; hsa-miR-330- | 0.681 | 0.547 | 0.815 | 0.400 | 0.744 | 0.214 |
| | 5p/hsa-miR-550a-3-5p-hsa-miR-550a-5p; hsa-miR- | | | | | | |
| | 378g/hsa-miR-485-5p; hsa-miR-501-3p/hsa-miR- | | | | | | |
| | 4446-3p; hsa-miR-30c-5p/hsa-miR-7-5p; hsa-miR- | | | | | | |
| | 20a-5p/hsa-miR-486-3p | | | | | | |
| 18 | hsa-miR-7-5p/hsa-miR-485-5p; hsa-miR-323b- | 0.65 | 0.518 | 0.783 | 0.300 | 0.638 | 0.270 |
| | 3p/hsa-mir-320c-2; hsa-miR-99b-3p/hsa-miR-340- | | | | | | |
| | 5p; hsa-miR-221-5p/hsa-miR-760 | | | | | | |
| 19 | hsa-miR-7-5p/hsa-miR-485-5p; hsa-miR-1306- | 0.683 | 0.553 | 0.814 | 0.433 | 0.598 | 0.229 |
| | 3p/hsa-miR-664a-5p; hsa-miR-433-3p/hsa-miR- | | | | | | |
| | 345-5p; hsa-miR-221-5p/hsa-miR-760 | | | | | | |
| 20 | hsa-miR-7-5p/hsa-miR-485-5p; hsa-miR-330- | 0.684 | 0.56 | 0.808 | 0.462 | 0.561 | 0.233 |
| | 5p/hsa-miR-550a-3-5p-hsa-miR-550a-5p; hsa-miR- | | | | | | |
| | 1306-3p/hsa-miR-1224-5p | | | | | | |
| 21 | hsa-miR-7-5p/hsa-miR-485-5p; hsa-miR-1306- | 0.672 | 0.54 | 0.804 | 0.391 | 0.523 | 0.158 |
| | 3p/hsa-miR-664a-5p; hsa-miR-99b-3p/hsa-miR- | | | | | | |
| | 760; hsa-miR-7-5p/hsa-miR-4446-3p | | | | | | |
| 22 | hsa-miR-7-5p/hsa-miR-485-5p; hsa-miR-221- | 0.672 | 0.548 | 0.796 | 0.425 | 0.459 | 0.151 |
| | 5p/hsa-miR-760; hsa-miR-1260b/hsa-mir-320c-2; | | | | | | |
| | hsa-miR-1306-3p/hsa-miR-664a-5p | | | | | | |
| 23 | hsa-miR-1306-3p/hsa-miR-664a-5p; hsa-miR- | 0.678 | 0.552 | 0.804 | 0.442 | 0.434 | 0.172 |
| | 378g/hsa-miR-485-5p; hsa-miR-30c-5p/hsa-miR- | | | | | | |
| | 100-5p; hsa-miR-501-3p/hsa-miR-484; hsa-miR- | | | | | | |
| | 221-5p/hsa-miR-760 | | | | | | |
| 24 | hsa-miR-7-5p/hsa-miR-485-5p; hsa-miR-221- | 0.654 | 0.52 | 0.787 | 0.327 | 0.387 | 0.192 |
| | 5p/hsa-miR-760; hsa-miR-1306-3p/hsa-miR-148a- | | | | | | |
| | 5p; hsa-miR-30c-5p/hsa-miR-320b; hsa-miR- | | | | | | |
| | 378e/hsa-miR-140-5p | | | | | | |
| 25 | hsa-miR-30c-5p/hsa-miR-27b-3p; hsa-miR-199a- | 0.624 | 0.507 | 0.742 | 0.293 | 0.190 | 0.297 |
| | 3p-hsa-miR-199b-3p/hsa-miR-485-5p; hsa-miR- | | | | | | |
| | 1306-3p/hsa-miR-664a-5p | | | | | | |
| 26 | hsa-miR-7-5p/hsa-miR-485-5p; hsa-miR-30c- | 0.661 | 0.552 | 0.77 | 0.462 | 0.172 | 0.121 |
| | 5p/hsa-miR-125a-5p; hsa-miR-1-3p/hsa-miR-345- | | | | | | |
| | 5p | | | | | | |
| 27 | hsa-miR-7-5p/hsa-miR-485-5p; hsa-miR-1-3p/hsa- | 0.635 | 0.516 | 0.755 | 0.333 | 0.108 | 0.142 |
| | miR-345-5p; hsa-miR-99b-3p/hsa-miR-483-3p; | | | | | | |
| | hsa-miR-30c-5p/hsa-miR-10b-5p | | | | | | |
| 28 | hsa-miR-1306-3p/hsa-miR-664a-5p; hsa-miR-7- | 0.649 | 0.527 | 0.771 | 0.378 | 0.055 | 0.117 |
| | 5p/hsa-miR-485-5p; hsa-miR-433-3p/hsa-miR-345- | | | | | | |
| | 5p; hsa-miR-30c-5p/hsa-miR-27b-3p; | | | | | | |
| | hsa-miR-99b-3p/hsa-miR-148a-5p | | | | | | |
| 29 | hsa-miR-7-5p/hsa-miR-485-5p; hsa-miR-1306- | 0.659 | 0.527 | 0.79 | 0.382 | −0.003 | 0.135 |
| | 3p/hsa-miR-664a-5p; hsa-miR-1-3p/hsa-miR-345- | | | | | | |
| | 5p; hsa-miR-99b-3p/hsa-miR-518e-5p-hsa-miR- | | | | | | |
| | 519a-5p-hsa-miR-519b-5p-hsa-miR-519c-5p-hsa- | | | | | | |
| | miR-522-5p-hsa-miR-523-5p | | | | | | |
| 30 | hsa-miR-7-5p/hsa-miR-485-5p; hsa-miR-1306- | 0.647 | 0.526 | 0.767 | 0.387 | −0.111 | 0.106 |
| | 3p/hsa-miR-664a-5p; hsa-miR-99b-3p/hsa-miR- | | | | | | |
| | 760; hsa-miR-760/hsa-miR-345-5p | | | | | | |
| 31 | hsa-miR-7-5p/hsa-miR-485-5p; hsa-miR-378e/hsa- | 0.66 | 0.521 | 0.798 | 0.376 | −0.211 | 0.146 |
| | miR-483-3p; hsa-miR-194-5p/hsa-miR-486-3p; | | | | | | |
| | hsa-miR-26b-5p/hsa-miR-769-5p; hsa-miR-99b- | | | | | | |
| | 3p/hsa-miR-760; hsa-miR-99b-3p/hsa-miR-144-5p; | | | | | | |
| | hsa-miR-221-5p/hsa-miR-197-3p | | | | | | |
| 32 | hsa-miR-1306-3p/hsa-miR-664a-5p; hsa-miR- | 0.651 | 0.518 | 0.784 | 0.367 | −0.243 | 0.132 |
| | 378g/hsa-miR-485-5p; hsa-miR-628-3p/hsa-miR- | | | | | | |
| | 19b-3p; hsa-miR-221-5p/hsa-miR-760 | | | | | | |
| 33 | hsa-miR-7-5p/hsa-miR-485-5p; hsa-miR- | 0.619 | 0.504 | 0.734 | 0.321 | −0.313 | 0.188 |
| | 1260b/hsa-mir-320c-2; hsa-miR-433-3p/hsa-miR- | | | | | | |
| | 345-5p | | | | | | |
| 34 | hsa-miR-7-5p/hsa-miR-485-5p; hsa-miR-330- | 0.636 | 0.505 | 0.767 | 0.325 | −0.318 | 0.174 |
| | 5p/hsa-miR-550a-3-5p-hsa-miR-550a-5p; hsa-miR- | | | | | | |
| | 1-3p/hsa-miR-345-5p; hsa-miR-760/hsa-miR-345- | | | | | | |
| | 5p | | | | | | |
| 35 | hsa-miR-1306-3p/hsa-miR-664a-5p; hsa-miR-7- | 0.677 | 0.535 | 0.819 | 0.443 | −0.409 | 0.215 |
| | 5p/hsa-miR-485-5p; hsa-miR-760/hsa-miR-345-5p; | | | | | | |
| | hsa-miR-221-5p/hsa-miR-1908-5p; hsa-miR-30c- | | | | | | |
| | 5p/hsa-miR-7-5p | | | | | | |
| 36 | hsa-miR-7-5p/hsa-miR-485-5p; hsa-miR-1306- | 0.629 | 0.509 | 0.748 | 0.347 | −0.414 | 0.126 |
| | 3p/hsa-miR-1224-5p; hsa-miR-99b-3p/hsa-miR- | | | | | | |
| | 760; hsa-miR-543/hsa-miR-345-5p | | | | | | |
| 37 | hsa-miR-7-5p/hsa-miR-485-5p; hsa-miR-330- | 0.661 | 0.553 | 0.77 | 0.515 | −0.478 | 0.124 |
| | 5p/hsa-miR-550a-3-5p-hsa-miR-550a-5p; hsa-miR- | | | | | | |
| | 30c-5p/hsa-miR-7-5p | | | | | | |
| 38 | hsa-miR-7-5p/hsa-miR-485-5p; hsa-miR-330- | 0.635 | 0.507 | 0.763 | 0.345 | −0.484 | 0.133 |
| | 5p/hsa-miR-550a-3-5p-hsa-miR-550a-5p; hsa-miR- | | | | | | |
| | 1-3p/hsa-miR-345-5p | | | | | | |
| 39 | hsa-miR-98-5p/hsa-miR-485-5p; hsa-miR-330- | 0.637 | 0.514 | 0.761 | 0.376 | −0.550 | 0.105 |
| | 5p/hsa-miR-550a-3-5p-hsa-miR-550a-5p; hsa-miR- | | | | | | |
| | 30c-5p/hsa-miR-10b-5p; hsa-miR-433-3p/hsa-miR- | | | | | | |
| | 345-5p | | | | | | |
| 40 | hsa-miR-7-5p/hsa-miR-485-5p; hsa-miR-1306- | 0.642 | 0.511 | 0.773 | 0.365 | −0.555 | 0.120 |
| | 3p/hsa-miR-664a-5p; hsa-miR-1-3p/hsa-miR-345- | | | | | | |
| | 5p; hsa-miR-628-3p/hsa-miR-19b-3p | | | | | | |
| 41 | hsa-miR-7-5p/hsa-miR-485-5p; hsa-miR-1306- | 0.654 | 0.521 | 0.788 | 0.403 | −0.566 | 0.113 |
| | 3p/hsa-miR-664a-5p; hsa-miR-99b-3p/hsa-miR- | | | | | | |
| | 760; hsa-miR-323b-3p/hsa-mir-320c-2 | | | | | | |
| 42 | hsa-miR-7-5p/hsa-miR-485-5p; hsa-miR-221- | 0.635 | 0.501 | 0.77 | 0.330 | −0.589 | 0.149 |
| | 5p/hsa-miR-760; hsa-miR-330-5p/hsa-miR-550a-3- | | | | | | |
| | 5p-hsa-miR-550a-5p; hsa-miR-1306-3p/hsa-miR- | | | | | | |
| | 664a-5p; hsa-miR-766-3p/hsa-miR-148a-5p | | | | | | |
| 43 | hsa-miR-7-5p/hsa-miR-485-5p; hsa-miR-1306- | 0.646 | 0.514 | 0.778 | 0.388 | −0.709 | 0.101 |
| | 3p/hsa-miR-664a-5p; hsa-miR-99b-3p/hsa-miR- | | | | | | |
| | 760; hsa-miR-7-5p/hsa-let-7c-5p | | | | | | |
| 44 | hsa-miR-30c-5p/hsa-miR-27b-3p; hsa-miR-7- | 0.638 | 0.507 | 0.769 | 0.362 | −0.716 | 0.119 |
| | 5p/hsa-miR-485-5p; hsa-miR-1260b/hsa-mir-320c- | | | | | | |
| | 2; hsa-miR-433-3p/hsa-miR-345-5p | | | | | | |
| 45 | hsa-miR-7-5p/hsa-miR-485-5p; hsa-miR-511- | 0.703 | 0.564 | 0.843 | 0.576 | −0.749 | 0.415 |
| | 5p/hsa-miR-148a-5p; hsa-miR-23b-5p/hsa-miR- | | | | | | |
| | 2110; hsa-miR-221-5p/hsa-miR-760; hsa-miR- | | | | | | |
| | 323b-3p/hsa-mir-320c-2; hsa-miR-20a-5p/hsa-miR- | | | | | | |
| | 486-3p | | | | | | |
| 46 | hsa-miR-7-5p/hsa-miR-485-5p; hsa-miR-221- | 0.657 | 0.526 | 0.788 | 0.441 | −0.826 | 0.113 |
| | 5p/hsa-miR-760; hsa-miR-15b-5p/hsa-miR-197-3p; | | | | | | |
| | hsa-miR-194-5p/hsa-miR-486-3p; hsa-miR-1306- | | | | | | |
| | 3p/hsa-miR-335-3p | | | | | | |
| 47 | hsa-miR-98-5p/hsa-miR-485-5p; hsa-miR-1306- | 0.672 | 0.535 | 0.808 | 0.490 | −1.035 | 0.145 |
| | 3p/hsa-miR-664a-5p; hsa-miR-99b-3p/hsa-miR- | | | | | | |
| | 485-5p; hsa-miR-221-5p/hsa-miR-197-3p | | | | | | |
| 48 | hsa-miR-30c-5p/hsa-miR-27b-3p; hsa-miR-221- | 0.676 | 0.541 | 0.812 | 0.516 | −1.084 | 0.163 |
| | 5p/hsa-miR-197-3p; hsa-miR-1306-3p/hsa-miR- | | | | | | |
| | 150-3p; hsa-miR-15b-5p/hsa-miR-197-3p | | | | | | |
| 49 | hsa-miR-7-5p/hsa-miR-485-5p; hsa-miR-330- | 0.652 | 0.528 | 0.775 | 0.479 | −1.234 | 0.097 |
| | 5p/hsa-miR-550a-3-5p-hsa-miR-550a-5p; hsa-miR- | | | | | | |
| | 30c-5p/hsa-miR-21-5p | | | | | | |
| 50 | hsa-miR-7-5p/hsa-miR-485-5p; hsa-miR-99b- | 0.682 | 0.546 | 0.818 | 0.546 | 1.238 | 0.216 |
| | 3p/hsa-miR-483-3p; hsa-miR-330-5p/hsa-miR- | | | | | | |
| | 550a-3-5p-hsa-miR-550a-5p; hsa-miR-30c-5p/hsa- | | | | | | |
| | miR-100-5p; hsa-miR-30c-5p/hsa-miR-425-5p; | | | | | | |
| | hsa-miR-511-5p/hsa-miR-148a-5p | | | | | | |
| 51 | hsa-miR-30c-5p/hsa-miR-27b-3p; hsa-miR-7- | 0.65 | 0.503 | 0.798 | 0.388 | 1.257 | 0.116 |
| | 5p/hsa-miR-4446-3p; hsa-miR-1260b/hsa-mir- | | | | | | |
| | 320c-2; hsa-miR-1260b/hsa-mir-320c-2; hsa-miR- | | | | | | |
| | 1260b/hsa-mir-320c-2 | | | | | | |
| 52 | hsa-miR-7-5p/hsa-miR-485-5p; hsa-miR-330- | 0.645 | 0.519 | 0.771 | 0.456 | −1.366 | 0.083 |
| | 5p/hsa-miR-550a-3-5p-hsa-miR-550a-5p; hsa-miR- | | | | | | |
| | 30c-5p/hsa-let-7g-5p | | | | | | |
| 53 | hsa-miR-7-5p/hsa-miR-485-5p; hsa-miR-1306- | 0.644 | 0.518 | 0.77 | 0.454 | −1.393 | 0.081 |
| | 3p/hsa-miR-1224-5p; hsa-miR-142-5p/hsa-miR- | | | | | | |
| | 485-5p; hsa-miR-221-5p/hsa-miR-197-3p | | | | | | |
| 54 | hsa-miR-98-5p/hsa-miR-485-5p; hsa-miR-330- | 0.641 | 0.524 | 0.757 | 0.483 | −1.477 | 0.078 |
| | 5p/hsa-miR-550a-3-5p-hsa-miR-550a-5p; hsa-miR- | | | | | | |
| | 30c-5p/hsa-miR-625-3p | | | | | | |
| 55 | hsa-miR-7-5p/hsa-miR-485-5p; hsa-miR-23b- | 0.658 | 0.521 | 0.795 | 0.489 | −1.703 | 0.107 |
| | 5p/hsa-miR-2110; hsa-miR-30c-5p/hsa-miR-125a- | | | | | | |
| | 5p; hsa-miR-760/hsa-miR-345-5p; hsa-miR-99b- | | | | | | |
| | 3p/hsa-miR-150-3p | | | | | | |
| 56 | hsa-miR-1306-3p/hsa-miR-664a-5p; hsa-miR- | 0.644 | 0.505 | 0.783 | 0.456 | −2.055 | 0.079 |
| | 378g/hsa-miR-485-5p; hsa-miR-30c-5p/hsa-miR- | | | | | | |
| | 27b-3p; hsa-miR-766-3p/hsa-miR-148a-5p; hsa- | | | | | | |
| | miR-99b-3p/hsa-miR-518e-5p-hsa-miR-519a-5p- | | | | | | |
| | hsa-miR-519b-5p-hsa-miR-519c-5p-hsa-miR-522- | | | | | | |
| | 5p-hsa-miR-523-5p | | | | | | |
| 57 | hsa-miR-7-5p/hsa-miR-485-5p; hsa-miR-221- | 0.641 | 0.503 | 0.78 | 0.456 | −2.147 | 0.077 |
| | 5p/hsa-miR-760; hsa-miR-99b-3p/hsa-miR-340-5p; | | | | | | |
| | hsa-miR-1306-3p/hsa-miR-150-3p | | | | | | |
| 58 | hsa-miR-7-5p/hsa-miR-485-5p; hsa-miR-330- | 0.638 | 0.506 | 0.77 | 0.476 | −2.264 | 0.071 |
| | 5p/hsa-miR-550a-3-5p-hsa-miR-550a-5p; hsa-miR- | | | | | | |
| | 323b-3p/hsa-mir-320c-2 | | | | | | |
| 59 | hsa-miR-1306-3p/hsa-miR-664a-5p; hsa-miR-148b- | 0.644 | 0.504 | 0.785 | 0.483 | −2.453 | 0.080 |
| | 3p/hsa-miR-941; hsa-miR-378g/hsa-miR-485-5p; | | | | | | |
| | hsa-miR-99b-3p/hsa-miR-760; hsa-miR-221- | | | | | | |
| | 5p/hsa-miR-760; hsa-miR-30c-5p/hsa-miR-27b-3p | | | | | | |
| 60 | hsa-miR-221-5p/hsa-miR-760; hsa-miR-330- | 0.647 | 0.508 | 0.786 | 0.535 | −2.948 | 0.083 |
| | 5p/hsa-miR-550a-3-5p-hsa-miR-550a-5p; hsa-miR- | | | | | | |
| | 501-3p/hsa-miR-485-5p; hsa-miR-99b-3p/hsa-miR- | | | | | | |
| | 518e-5p-hsa-miR-519a-5p-hsa-miR-519b-5p-hsa- | | | | | | |
| | miR-519c-5p-hsa-miR-522-5p-hsa-miR-523-5p | | | | | | |
| 61 | hsa-miR-330-5p/hsa-miR-223-3p; hsa-miR-7- | 0.629 | 0.502 | 0.755 | 0.520 | −3.035 | 0.061 |
| | 5p/hsa-miR-485-5p; hsa-miR-511-5p/hsa-miR- | | | | | | |
| | 148a-5p | | | | | | |
| 62 | hsa-miR-7-5p/hsa-miR-485-5p; hsa-miR-1306- | 0.64 | 0.506 | 0.774 | 0.535 | −3.037 | 0.070 |
| | 3p/hsa-miR-664a-5p; hsa-miR-30a-5p/hsa-miR- | | | | | | |
| | 941; hsa-miR-30c-5p/hsa-miR-27b-3p | | | | | | |

The panels of nucleic acid biomarker pairs listed in Table 6 can be used for determining a pregnant female's risk of developing placental dysfunction. The panels of nucleic acid biomarkers listed in Table 6 can also be used in a method of treating or preventing placental dysfunction in a pregnant female.

### Example 4: Determing Risk For Placental Dysfunction

This example illustrates a method for using an exemplary model described herein. For the purposes of illustration, this example tracks hypothetical subjects through an exemplary binary decision tree in chronological order, i.e., by sequentially analyzing subject reversal scores node by node. However, as a person skilled in the art will appreciate, actual subject risk scores as determined from the node which a subject is assigned occurs during a single analysis, i.e., the analyses of subject reversal scores at each node are performed near simultaneously.

Subjects are tested (results are represented by the binary decision tree illustrated in FIG. 1) as described herein. The miRNA levels of subjects are determined from an appropriate matrix, i.e. blood, serum, etc. Measurements of miRNA refer to the logarithm of the abundance of analyte in a given matrix. The ratio of miRNA log abundances is multiplied by gestational age at blood draw in days. The binary decision tree is used to determine risk of placental dysfunction as described below.

Making reference to the binary decision tree illustrated in FIG. 1, subjects in node 1 with a reversal score derived from a ratio of hsa-miR-98-5p/hsa-miR-485-5p that is above 273.1 will advance to terminal node 9 and those with a reversal score less than or equal to 273.1 will advance to node 2.

Subjects in node 2 will be evaluated by a reversal score derived from the ratio of nucleic acid biomarkers hsa-miR-1306-3p/hsa-miR-1224-5p. Subjects with reversal scores above 49.0 will advance to node 6 while subjects with reversal scores less than an equal to 49.0 will advance to node 3.

Subjects in node 6 will be evaluated by a reversal score derived from the ratio of hsa-miR-221-5p/hsa-miR-197-3p. Subjects in node 6 having a score above 69.8 will advance to terminal node 8 and subjects with a score less than or equal to 69.8 will advance to terminal node 7. Subjects in node 3 will be evaluated by their score derived from the ratio of hsa.miR.330.5p/hsa.miR.550a.3.5p.hsa.miR.550a.5p. Subjects with a reversal score above 140.9 will advance to terminal node 5 and subjects with a reversal score less than or equal to 140.9 will advance to terminal node 4. When treating this tree as a binary classifier, subjects in right-side terminal nodes are classified as cases and those in left-side terminal nodes are classified as controls.

All known performance statistics can be derived from the frequency-weighted average of the stratification in terminal nodes. Risk scores are assigned to an individual subject in any particular node based on the proportion of cases (subjections with placental dysfunction) amongst all subjects assigned to that node in the clinical validation set. For example, if node 7 in a clinical validation dataset that establishes test performance has 30 cases (subjections with placental dysfunction) and 70 controls (subjects without placental dysfunction), any subject stratified to that node will have a risk of 30%. This may assume that the original distribution of cases and controls in the study was at population prevalence. If node 9 in a clinical validation dataset that establishes test performance has 80 cases and 20 controls, any subject stratified to that node will have a risk of 80%. This may assume that the original distribution of cases and controls in the study was at population prevalence. If the clinical validation study data are not at population prevalence, the risk scores will be adjusted to prevalence by multiplying case proportions by the ratio of (population case prevalence)/(study case prevalence), and control proportions by the ratio of (population control prevalence)/(study control prevalence).

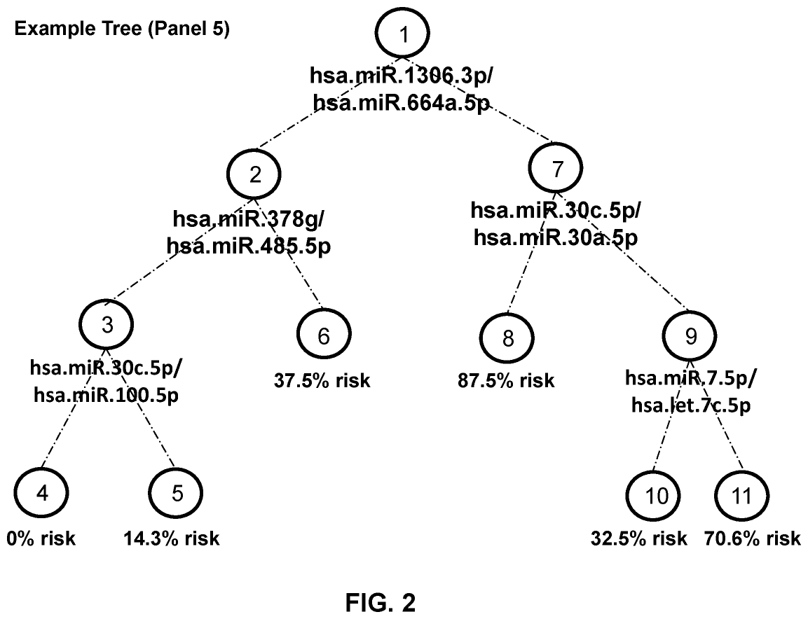

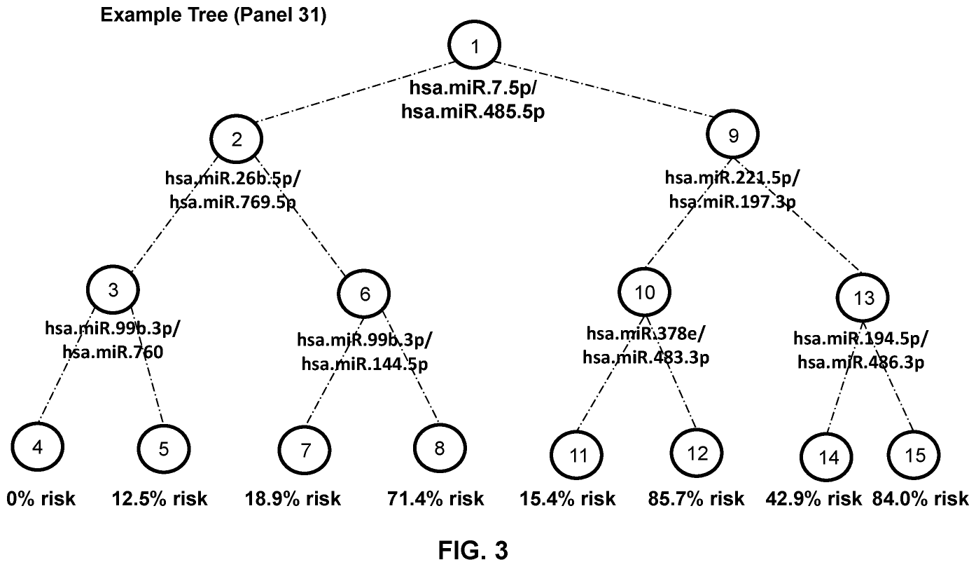

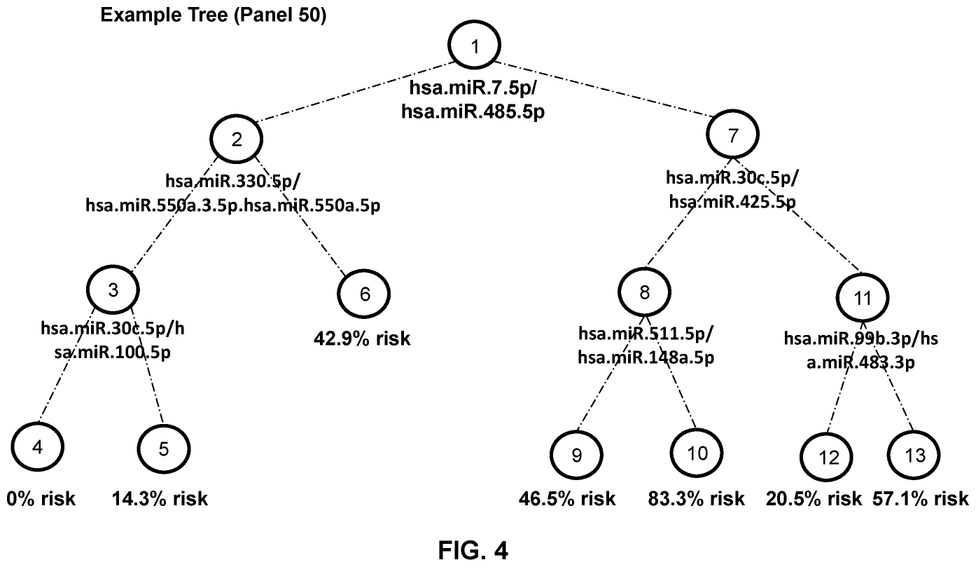

FIGS. 2-4 additional binary decision trees for determining risk of placental dysfunction. FIG. 2 shows a binary decision tree modeled using the panel of biomarkers identified as “panel 5” from the list of nucleic acid biomarker pairs listed in Table 6. FIG. 3 shows a binary decision tree modeled with the biomarkers identified as “panel 31” from Table 6. FIG. 4. Shows a binary decision tree modeled with biomarkers identified as “panel 50” from Table 6. Each illustrated binary tree has terminal nodes are annotated with certain risk scores that will be assigned to subjects associated with the corresponding terminal node. The trees are modeled to determine risk for placental dysfunction based on the panel of biomarkers based on the data below in Table 7.

TABLE 7
Data for modeling binary decision trees from biomarker panels.

| | | | | | | | | | | | pre- | pre- | |
|---|---|---|---|---|---|---|---|---|---|---|---|---|---|
| | | | | children. | | | | inhomo- | | rank_ | dict_ | dict_ | cut |
| Model | node | reversal | termini | L.R | AUC | lowerCI | UpperCI | geneity | Zsum | sum | L | R | off |
| 1 | 1 | hsa-miR- | 5 | 2, 9 | 0.746 | 0.626 | 0.865 | 0.479 | 3.563 | 2.018 | 0.307 | 0.818 | 273.1 |
| | | 98- | | | | | | | | | | | |
| | | 5p/hsa- | | | | | | | | | | | |
| | | miR-485- | | | | | | | | | | | |
| | | 5p | | | | | | | | | | | |
| 1 | 2 | hsa-miR- | 4 | 3, 6 | 0.746 | 0.626 | 0.865 | 0.479 | 3.563 | 2.018 | 0.104 | 0.416 | 49.0 |
| | | 1306- | | | | | | | | | | | |
| | | 3p/hsa- | | | | | | | | | | | |
| | | miR- | | | | | | | | | | | |
| | | 1224-5p | | | | | | | | | | | |
| 1 | 3 | hsa-miR- | 2 | 4, 5 | 0.746 | 0.626 | 0.865 | 0.479 | 3.563 | 2.018 | 0.0244 | 0.571 | 140.9 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 1 | 6 | hsa-miR- | 2 | 7, 8 | 0.746 | 0.626 | 0.865 | 0.479 | 3.563 | 2.018 | 0.312 | 0.68 | 69.8 |
| | | 221- | | | | | | | | | | | |
| | | 5p/hsa- | | | | | | | | | | | |
| | | miR-197- | | | | | | | | | | | |
| | | 3p | | | | | | | | | | | |
| 2 | 1 | hsa-miR- | 5 | 2, 9 | 0.735 | 0.62 | 0.85 | 0.456 | 3.562 | 1.187 | 0.299 | 0.786 | 330.8 |
| | | 7-5p/hsa- | | | | | | | | | | | |
| | | miR-485- | | | | | | | | | | | |
| | | 5p | | | | | | | | | | | |
| 2 | 2 | hsa-miR- | 4 | 3, 6 | 0.735 | 0.62 | 0.85 | 0.456 | 3.562 | 1.187 | 0.122 | 0.4 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 2 | 3 | hsa-miR- | 2 | 4, 5 | 0.735 | 0.62 | 0.85 | 0.456 | 3.562 | 1.187 | 0.0303 | 0.312 | 164.6 |
| | | 99b- | | | | | | | | | | | |
| | | 3p/hsa- | | | | | | | | | | | |
| | | miR-760 | | | | | | | | | | | |
| 2 | 6 | hsa-miR- | 2 | 7, 8 | 0.735 | 0.62 | 0.85 | 0.456 | 3.562 | 1.187 | 0.15 | 0.477 | 143.8 |
| | | 98- | | | | | | | | | | | |
| | | 5p/hsa- | | | | | | | | | | | |
| | | miR-483- | | | | | | | | | | | |
| | | 3p | | | | | | | | | | | |
| 3 | 1 | hsa-miR- | 5 | 2, 3 | 0.749 | 0.619 | 0.879 | 0.501 | 2.929 | 2.350 | 0.778 | 0.317 | 98.2 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 3 | 3 | hsa-miR- | 4 | 4, 7 | 0.749 | 0.619 | 0.879 | 0.501 | 2.929 | 2.350 | 0.52 | 0.202 | 88.3 |
| | | 433- | | | | | | | | | | | |
| | | 3p/hsa- | | | | | | | | | | | |
| | | miR-345- | | | | | | | | | | | |
| | | 5p | | | | | | | | | | | |
| 3 | 4 | hsa-miR- | 2 | 5, 6 | 0.749 | 0.619 | 0.879 | 0.501 | 2.929 | 2.350 | 0.382 | 0.812 | 80.6 |
| | | 15b- | | | | | | | | | | | |
| | | 5p/hsa- | | | | | | | | | | | |
| | | miR-197- | | | | | | | | | | | |
| | | 3p | | | | | | | | | | | |
| 3 | 7 | hsa-miR- | 2 | 8, 9 | 0.749 | 0.619 | 0.879 | 0.501 | 2.929 | 2.350 | 0.0862 | 0.419 | 263.6 |
| | | 363- | | | | | | | | | | | |
| | | 3p/hsa- | | | | | | | | | | | |
| | | miR-485- | | | | | | | | | | | |
| | | 5p | | | | | | | | | | | |
| 4 | 1 | hsa-miR- | 6 | 2, 5 | 0.685 | 0.548 | 0.821 | 0.259 | 2.651 | 0.762 | 0.0857 | 0.425 | 22.7 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 4 | 2 | hsa-miR- | 2 | 3, 4 | 0.685 | 0.548 | 0.821 | 0.259 | 2.651 | 0.762 | 0 | 0.429 | 187.3 |
| | | 142- | | | | | | | | | | | |
| | | 5p/hsa- | | | | | | | | | | | |
| | | miR-485- | | | | | | | | | | | |
| | | 5p | | | | | | | | | | | |
| 4 | 5 | hsa-miR- | 4 | 6, 9 | 0.685 | 0.548 | 0.821 | 0.259 | 2.651 | 0.762 | 0.138 | 0.524 | 34.3 |
| | | 15b- | | | | | | | | | | | |
| | | 5p/hsa- | | | | | | | | | | | |
| | | miR-197- | | | | | | | | | | | |
| | | 3p | | | | | | | | | | | |
| 4 | 6 | hsa-miR- | 2 | 7, 8 | 0.685 | 0.548 | 0.821 | 0.259 | 2.651 | 0.762 | 0 | 0.444 | 234.7 |
| | | 7-5p/hsa- | | | | | | | | | | | |
| | | miR-485- | | | | | | | | | | | |
| | | 5p | | | | | | | | | | | |
| 4 | 9 | hsa-miR- | 2 | 10, 11 | 0.685 | 0.548 | 0.821 | 0.259 | 2.651 | 0.762 | 1 | 0.42 | −80.7 |
| | | 433- | | | | | | | | | | | |
| | | 3p/hsa- | | | | | | | | | | | |
| | | miR-345- | | | | | | | | | | | |
| | | 5p | | | | | | | | | | | |
| 5 | 1 | hsa-miR- | 6 | 2, 7 | 0.696 | 0.59 | 0.802 | 0.420 | 2.573 | 0.396 | 0.152 | 0.431 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 5 | 2 | hsa-miR- | 3 | 3, 6 | 0.696 | 0.59 | 0.802 | 0.420 | 2.573 | 0.396 | 0.0333 | 0.375 | 101.8 |
| | | 378g/hsa- | | | | | | | | | | | |
| | | miR-485- | | | | | | | | | | | |
| | | 5p | | | | | | | | | | | |
| 5 | 3 | hsa-miR- | 2 | 4, 5 | 0.696 | 0.59 | 0.802 | 0.420 | 2.573 | 0.396 | 0 | 0.143 | 141.6 |
| | | 30c- | | | | | | | | | | | |
| | | 5p/hsa- | | | | | | | | | | | |
| | | miR-100- | | | | | | | | | | | |
| | | 5p | | | | | | | | | | | |
| 5 | 7 | hsa-miR- | 3 | 8, 9 | 0.696 | 0.59 | 0.802 | 0.420 | 2.573 | 0.396 | 0.875 | 0.394 | 81.8 |
| | | 30c- | | | | | | | | | | | |
| | | 5p/hsa- | | | | | | | | | | | |
| | | miR-30a- | | | | | | | | | | | |
| | | 5p | | | | | | | | | | | |
| 5 | 9 | hsa-miR- | 2 | 10, 11 | 0.696 | 0.59 | 0.802 | 0.420 | 2.573 | 0.396 | 0.325 | 0.706 | 186.8 |
| | | 7-5p/hsa- | | | | | | | | | | | |
| | | let-7c-5p | | | | | | | | | | | |
| 6 | 1 | hsa-miR- | 5 | 2, 9 | 0.719 | 0.593 | 0.845 | 0.442 | 2.437 | 0.774 | 0.282 | 0.824 | 330.8 |
| | | 7-5p/hsa- | | | | | | | | | | | |
| | | miR-485- | | | | | | | | | | | |
| | | 5p | | | | | | | | | | | |
| 6 | 2 | hsa-miR- | 4 | 3, 6 | 0.719 | 0.593 | 0.845 | 0.442 | 2.437 | 0.774 | 0.19 | 0.447 | 88.5 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 6 | 3 | hsa-miR- | 2 | 4, 5 | 0.719 | 0.593 | 0.845 | 0.442 | 2.437 | 0.774 | 0.298 | 0.0541 | 138.2 |
| | | 433- | | | | | | | | | | | |
| | | 3p/hsa- | | | | | | | | | | | |
| | | miR-345- | | | | | | | | | | | |
| | | 5p | | | | | | | | | | | |
| 6 | 6 | hsa-miR- | 2 | 7, 8 | 0.719 | 0.593 | 0.845 | 0.442 | 2.437 | 0.774 | 0.654 | 0.19 | 169.2 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 7 | 1 | hsa-miR- | 5 | 2, 9 | 0.65 | 0.524 | 0.776 | 0.212 | 2.091 | 1.073 | 0.292 | 1 | 359.8 |
| | | 7-5p/hsa- | | | | | | | | | | | |
| | | miR-485- | | | | | | | | | | | |
| | | 5p | | | | | | | | | | | |
| 7 | 2 | hsa-miR- | 4 | 3, 6 | 0.65 | 0.524 | 0.776 | 0.212 | 2.091 | 1.073 | 0.087 | 0.396 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 7 | 3 | hsa-miR- | 2 | 4, 5 | 0.65 | 0.524 | 0.776 | 0.212 | 2.091 | 1.073 | 0 | 0.129 | 168.1 |
| | | 7-5p/hsa- | | | | | | | | | | | |
| | | miR- | | | | | | | | | | | |
| | | 4446-3p | | | | | | | | | | | |
| 7 | 6 | hsa-miR- | 2 | 7, 8 | 0.65 | 0.524 | 0.776 | 0.212 | 2.091 | 1.073 | 0.341 | 0.889 | 299.7 |
| | | 221- | | | | | | | | | | | |
| | | 5p/hsa- | | | | | | | | | | | |
| | | miR-760 | | | | | | | | | | | |
| 8 | 1 | hsa-miR- | 4 | 2, 7 | 0.684 | 0.576 | 0.792 | 0.408 | 2.052 | 0.286 | 0.282 | 0.824 | 306.5 |
| | | 7-5p/hsa- | | | | | | | | | | | |
| | | miR-485- | | | | | | | | | | | |
| | | 5p | | | | | | | | | | | |
| 8 | 2 | hsa-miR- | 3 | 3, 4 | 0.684 | 0.576 | 0.792 | 0.408 | 2.052 | 0.286 | 0.0667 | 0.347 | 22.7 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 8 | 4 | hsa-miR- | 2 | 5, 6 | 0.684 | 0.576 | 0.792 | 0.408 | 2.052 | 0.286 | 0.3 | 0.727 | 155.6 |
| | | 501- | | | | | | | | | | | |
| | | 3p/hsa- | | | | | | | | | | | |
| | | miR-484 | | | | | | | | | | | |
| 9 | 1 | hsa-miR- | 5 | 2, 9 | 0.704 | 0.574 | 0.834 | 0.377 | 2.016 | 0.482 | 0.279 | 0.789 | 300.1 |
| | | 7-5p/hsa- | | | | | | | | | | | |
| | | miR-485- | | | | | | | | | | | |
| | | 5p | | | | | | | | | | | |
| 9 | 2 | hsa-miR- | 4 | 3, 6 | 0.704 | 0.574 | 0.834 | 0.377 | 2.016 | 0.482 | 0.0638 | 0.402 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 9 | 3 | hsa-miR- | 2 | 4, 5 | 0.704 | 0.574 | 0.834 | 0.377 | 2.016 | 0.482 | 0 | 0.2 | 164.6 |
| | | 99b- | | | | | | | | | | | |
| | | 3p/hsa- | | | | | | | | | | | |
| | | miR-760 | | | | | | | | | | | |
| 9 | 6 | hsa-miR- | 2 | 7, 8 | 0.704 | 0.574 | 0.834 | 0.377 | 2.016 | 0.482 | 0.313 | 0.8 | 146.1 |
| | | 221- | | | | | | | | | | | |
| | | 5p/hsa- | | | | | | | | | | | |
| | | miR-760 | | | | | | | | | | | |
| 10 | 1 | hsa-miR- | 5 | 2, 9 | 0.677 | 0.565 | 0.79 | 0.382 | 1.860 | 0.223 | 0.271 | 0.842 | 306.5 |
| | | 7-5p/hsa- | | | | | | | | | | | |
| | | miR-485- | | | | | | | | | | | |
| | | 5p | | | | | | | | | | | |
| 10 | 2 | hsa-miR- | 4 | 3, 6 | 0.677 | 0.565 | 0.79 | 0.382 | 1.860 | 0.223 | 0.109 | 0.361 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 10 | 3 | hsa-miR- | 2 | 4, 5 | 0.677 | 0.565 | 0.79 | 0.382 | 1.860 | 0.223 | 0 | 0.312 | 159.2 |
| | | 99b- | | | | | | | | | | | |
| | | 3p/hsa- | | | | | | | | | | | |
| | | miR-379- | | | | | | | | | | | |
| | | 5p | | | | | | | | | | | |
| 10 | 6 | hsa-miR- | 2 | 7, 8 | 0.677 | 0.565 | 0.79 | 0.382 | 1.860 | 0.223 | 0.275 | 0.786 | 90.3 |
| | | 221- | | | | | | | | | | | |
| | | 5p/hsa- | | | | | | | | | | | |
| | | miR-197- | | | | | | | | | | | |
| | | 3p | | | | | | | | | | | |
| 11 | 1 | hsa-miR- | 5 | 2, 9 | 0.686 | 0.566 | 0.806 | 0.396 | 1.718 | 0.293 | 0.299 | 0.909 | 359.8 |
| | | 7-5p/hsa- | | | | | | | | | | | |
| | | miR-485- | | | | | | | | | | | |
| | | 5p | | | | | | | | | | | |
| 11 | 2 | hsa-miR- | 4 | 3, 6 | 0.686 | 0.566 | 0.806 | 0.396 | 1.718 | 0.293 | 0.382 | 0.0571 | 260.7 |
| | | 1-3p/hsa- | | | | | | | | | | | |
| | | miR-345- | | | | | | | | | | | |
| | | 5p | | | | | | | | | | | |
| 11 | 3 | hsa-miR- | 2 | 4, 5 | 0.686 | 0.566 | 0.806 | 0.396 | 1.718 | 0.293 | 0.286 | 0.68 | 130.8 |
| | | 99b- | | | | | | | | | | | |
| | | 3p/hsa- | | | | | | | | | | | |
| | | miR-150- | | | | | | | | | | | |
| | | 3p | | | | | | | | | | | |
| 11 | 6 | hsa-miR- | 2 | 7, 8 | 0.686 | 0.566 | 0.806 | 0.396 | 1.718 | 0.293 | 0.286 | 0 | 105.5 |
| | | 30c- | | | | | | | | | | | |
| | | 5p/hsa- | | | | | | | | | | | |
| | | miR- | | | | | | | | | | | |
| | | 146a-5p | | | | | | | | | | | |
| 12 | 1 | hsa-miR- | 4 | 2, 7 | 0.664 | 0.542 | 0.786 | 0.309 | 1.696 | 0.268 | 0.302 | 1 | 359.8 |
| | | 7-5p/hsa- | | | | | | | | | | | |
| | | miR-485- | | | | | | | | | | | |
| | | 5p | | | | | | | | | | | |
| 12 | 2 | hsa-miR- | 3 | 3, 4 | 0.664 | 0.542 | 0.786 | 0.309 | 1.696 | 0.268 | 0.0667 | 0.367 | 22.7 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 12 | 4 | hsa-miR- | 2 | 5, 6 | 0.664 | 0.542 | 0.786 | 0.309 | 1.696 | 0.268 | 0.301 | 0.75 | 163.9 |
| | | 628- | | | | | | | | | | | |
| | | 3p/hsa- | | | | | | | | | | | |
| | | miR-19b- | | | | | | | | | | | |
| | | 3p | | | | | | | | | | | |
| 13 | 1 | hsa-miR- | 5 | 2, 9 | 0.68 | 0.546 | 0.813 | 0.362 | 1.193 | 0.236 | 0.286 | 0.867 | 330.8 |
| | | 7-5p/hsa- | | | | | | | | | | | |
| | | miR-485- | | | | | | | | | | | |
| | | 5p | | | | | | | | | | | |
| 13 | 2 | hsa-miR- | 4 | 3, 6 | 0.68 | 0.546 | 0.813 | 0.362 | 1.193 | 0.236 | 0.0312 | 0.366 | 31.7 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 13 | 3 | hsa-miR- | 2 | 4, 5 | 0.68 | 0.546 | 0.813 | 0.362 | 1.193 | 0.236 | 0 | 0.143 | 182.4 |
| | | 30c- | | | | | | | | | | | |
| | | 5p/hsa- | | | | | | | | | | | |
| | | miR-7-5p | | | | | | | | | | | |
| 13 | 6 | hsa-miR- | 2 | 7, 8 | 0.68 | 0.546 | 0.813 | 0.362 | 1.193 | 0.236 | 0.263 | 0.68 | 137.2 |
| | | 221- | | | | | | | | | | | |
| | | 5p/hsa- | | | | | | | | | | | |
| | | miR-760 | | | | | | | | | | | |
| 14 | 1 | hsa-miR- | 7 | 2, 7 | 0.682 | 0.554 | 0.811 | 0.402 | 1.060 | 0.214 | 0.119 | 0.434 | 172.5 |
| | | 7-5p/hsa- | | | | | | | | | | | |
| | | miR-485- | | | | | | | | | | | |
| | | 5p | | | | | | | | | | | |
| 14 | 2 | hsa-miR- | 3 | 3, 6 | 0.682 | 0.554 | 0.811 | 0.402 | 1.060 | 0.214 | 0.0323 | 0.364 | 135.4 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 14 | 3 | hsa-let-7i- | 2 | 4, 5 | 0.682 | 0.554 | 0.811 | 0.402 | 1.060 | 0.214 | 0 | 0.143 | 180.3 |
| | | 5p/hsa- | | | | | | | | | | | |
| | | miR-191- | | | | | | | | | | | |
| | | 5p | | | | | | | | | | | |
| 14 | 7 | hsa-miR- | 4 | 8, 11 | 0.682 | 0.554 | 0.811 | 0.402 | 1.060 | 0.214 | 0.731 | 0.338 | 101.2 |
| | | 760/hsa- | | | | | | | | | | | |
| | | miR-345- | | | | | | | | | | | |
| | | 5p | | | | | | | | | | | |
| 14 | 8 | hsa-miR- | 2 | 9, 10 | 0.682 | 0.554 | 0.811 | 0.402 | 1.060 | 0.214 | 0.462 | 1 | 91.3 |
| | | 125a- | | | | | | | | | | | |
| | | 3p/hsa- | | | | | | | | | | | |
| | | miR-379- | | | | | | | | | | | |
| | | 5p | | | | | | | | | | | |
| 14 | 11 | hsa-miR- | 2 | 12, 13 | 0.682 | 0.554 | 0.811 | 0.402 | 1.060 | 0.214 | 0.269 | 0.692 | 317.1 |
| | | 7-5p/hsa- | | | | | | | | | | | |
| | | miR- | | | | | | | | | | | |
| | | 4446-3p | | | | | | | | | | | |
| 15 | 1 | hsa-miR- | 5 | 2, 9 | 0.671 | 0.558 | 0.784 | 0.427 | 0.922 | 0.161 | 0.304 | 0.9 | 295.2 |
| | | 98- | | | | | | | | | | | |
| | | 5p/hsa- | | | | | | | | | | | |
| | | miR-485- | | | | | | | | | | | |
| | | 5p | | | | | | | | | | | |
| 15 | 2 | hsa-miR- | 4 | 3, 6 | 0.671 | 0.558 | 0.784 | 0.427 | 0.922 | 0.161 | 0.128 | 0.396 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 15 | 3 | hsa-miR- | 2 | 4, 5 | 0.671 | 0.558 | 0.784 | 0.427 | 0.922 | 0.161 | 0.0513 | 0.5 | 83.2 |
| | | 99b- | | | | | | | | | | | |
| | | 3p/hsa- | | | | | | | | | | | |
| | | miR-22- | | | | | | | | | | | |
| | | 3p | | | | | | | | | | | |
| 15 | 6 | hsa-miR- | 2 | 7, 8 | 0.671 | 0.558 | 0.784 | 0.427 | 0.922 | 0.161 | 0.329 | 0.833 | 299.7 |
| | | 221- | | | | | | | | | | | |
| | | 5p/hsa- | | | | | | | | | | | |
| | | miR-760 | | | | | | | | | | | |
| 16 | 1 | hsa-miR- | 5 | 2, 9 | 0.649 | 0.516 | 0.782 | 0.282 | 0.780 | 0.399 | 0.296 | 0.846 | 318.6 |
| | | 7-5p/hsa- | | | | | | | | | | | |
| | | miR-485- | | | | | | | | | | | |
| | | 5p | | | | | | | | | | | |
| 16 | 2 | hsa-miR- | 4 | 3, 6 | 0.649 | 0.516 | 0.782 | 0.282 | 0.780 | 0.399 | 0.396 | 0.0909 | 168.1 |
| | | 433- | | | | | | | | | | | |
| | | 3p/hsa- | | | | | | | | | | | |
| | | miR-345- | | | | | | | | | | | |
| | | 5p | | | | | | | | | | | |
| 16 | 3 | hsa-miR- | 2 | 4, 5 | 0.649 | 0.516 | 0.782 | 0.282 | 0.780 | 0.399 | 0.329 | 1 | 106.6 |
| | | 15b- | | | | | | | | | | | |
| | | 5p/hsa- | | | | | | | | | | | |
| | | miR-197- | | | | | | | | | | | |
| | | 3p | | | | | | | | | | | |
| 16 | 6 | hsa-miR- | 2 | 7, 8 | 0.649 | 0.516 | 0.782 | 0.282 | 0.780 | 0.399 | 0 | 0.286 | 51.6 |
| | | 330- | | | | | | | | | | | |
| | | 5p/hsa- | | | | | | | | | | | |
| | | miR-223- | | | | | | | | | | | |
| | | 3p | | | | | | | | | | | |
| 17 | 1 | hsa-miR- | 7 | 2, 7 | 0.681 | 0.547 | 0.815 | 0.400 | 0.744 | 0.214 | 0.182 | 0.413 | 44.0 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 17 | 2 | hsa-miR- | 3 | 3, 6 | 0.681 | 0.547 | 0.815 | 0.400 | 0.744 | 0.214 | 0.0811 | 0.714 | 156.0 |
| | | 378g/hsa- | | | | | | | | | | | |
| | | miR-485- | | | | | | | | | | | |
| | | 5p | | | | | | | | | | | |
| 17 | 3 | hsa-miR- | 2 | 4, 5 | 0.681 | 0.547 | 0.815 | 0.400 | 0.744 | 0.214 | 0 | 0.136 | 157.6 |
| | | 501- | | | | | | | | | | | |
| | | 3p/hsa- | | | | | | | | | | | |
| | | miR- | | | | | | | | | | | |
| | | 4446-3p | | | | | | | | | | | |
| 17 | 7 | hsa-miR- | 4 | 8, 1 | 0.681 | 0.547 | 0.815 | 0.400 | 0.744 | 0.214 | 0.525 | 0.267 | 161.2 |
| | | 30c- | | | | | | | | | | | |
| | | 5p/hsa- | | | | | | | | | | | |
| | | miR-7-5p | | | | | | | | | | | |
| 17 | 8 | hsa-miR- | 2 | 9, 10 | 0.681 | 0.547 | 0.815 | 0.400 | 0.744 | 0.214 | 0.381 | 0.882 | 111.6 |
| | | 20a- | | | | | | | | | | | |
| | | 5p/hsa- | | | | | | | | | | | |
| | | miR-486- | | | | | | | | | | | |
| | | 3p | | | | | | | | | | | |
| 17 | 11 | hsa-miR- | 2 | 12, 13 | 0.681 | 0.547 | 0.815 | 0.400 | 0.744 | 0.214 | 0.143 | 0.7 | 151.0 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 18 | 1 | hsa-miR- | 5 | 2, 9 | 0.65 | 0.518 | 0.783 | 0.300 | 0.638 | 0.270 | 0.284 | 0.929 | 330.8 |
| | | 7-5p/hsa- | | | | | | | | | | | |
| | | miR-485- | | | | | | | | | | | |
| | | 5p | | | | | | | | | | | |
| 18 | 2 | hsa-miR- | 4 | 3, 6 | 0.65 | 0.518 | 0.783 | 0.300 | 0.638 | 0.270 | 0.159 | 0.481 | 154.5 |
| | | 323b- | | | | | | | | | | | |
| | | 3p/hsa- | | | | | | | | | | | |
| | | mir-320c- | | | | | | | | | | | |
| | | 2 | | | | | | | | | | | |
| 18 | 3 | hsa-miR- | 2 | 4, 5 | 0.65 | 0.518 | 0.783 | 0.300 | 0.638 | 0.270 | 0 | 0.213 | 63.6 |
| | | 99b- | | | | | | | | | | | |
| | | 3p/hsa- | | | | | | | | | | | |
| | | miR-340- | | | | | | | | | | | |
| | | 5p | | | | | | | | | | | |
| 18 | 6 | hsa-miR- | 2 | 7, 8 | 0.65 | 0.518 | 0.783 | 0.300 | 0.638 | 0.270 | 0.281 | 0.8 | 89.2 |
| | | 221- | | | | | | | | | | | |
| | | 5p/hsa- | | | | | | | | | | | |
| | | miR-760 | | | | | | | | | | | |
| 19 | 1 | hsa-miR- | 5 | 2, 9 | 0.683 | 0.553 | 0.814 | 0.433 | 0.598 | 0.229 | 0.279 | 0.789 | 300.1 |
| | | 7-5p/hsa- | | | | | | | | | | | |
| | | miR-485- | | | | | | | | | | | |
| | | 5p | | | | | | | | | | | |
| 19 | 2 | hsa-miR- | 4 | 3, 6 | 0.683 | 0.553 | 0.814 | 0.433 | 0.598 | 0.229 | 0.0732 | 0.375 | 51.6 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 19 | 3 | hsa-miR- | 2 | 4, 5 | 0.683 | 0.553 | 0.814 | 0.433 | 0.598 | 0.229 | 0.286 | 0.0294 | −100.7 |
| | | 433- | | | | | | | | | | | |
| | | 3p/hsa- | | | | | | | | | | | |
| | | miR-345- | | | | | | | | | | | |
| | | 5p | | | | | | | | | | | |
| 19 | 6 | hsa-miR- | 2 | 7, 8 | 0.683 | 0.553 | 0.814 | 0.433 | 0.598 | 0.229 | 0.292 | 0.75 | 156.3 |
| | | 221- | | | | | | | | | | | |
| | | 5p/hsa- | | | | | | | | | | | |
| | | miR-760 | | | | | | | | | | | |
| 20 | 1 | hsa-miR- | 4 | 2, 7 | 0.684 | 0.56 | 0.808 | 0.462 | 0.561 | 0.233 | 0.293 | 0.8 | 330.8 |
| | | 7-5p/hsa- | | | | | | | | | | | |
| | | miR-485- | | | | | | | | | | | |
| | | 5p | | | | | | | | | | | |
| 20 | 2 | hsa-miR- | 3 | 3, 6 | 0.684 | 0.56 | 0.808 | 0.462 | 0.561 | 0.233 | 0.233 | 0.706 | 151.0 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 20 | 3 | hsa-miR- | 2 | 4, 5 | 0.684 | 0.56 | 0.808 | 0.462 | 0.561 | 0.233 | 0.0385 | 0.391 | 63.5 |
| | | 1306- | | | | | | | | | | | |
| | | 3p/hsa- | | | | | | | | | | | |
| | | miR- | | | | | | | | | | | |
| | | 1224-5p | | | | | | | | | | | |
| 21 | 1 | hsa-miR- | 5 | 2, 9 | 0.672 | 0.54 | 0.804 | 0.391 | 0.523 | 0.158 | 0.304 | 0.9 | 359.8 |
| | | 7-5p/hsa- | | | | | | | | | | | |
| | | miR-485- | | | | | | | | | | | |
| | | 5p | | | | | | | | | | | |
| 21 | 2 | hsa-miR- | 4 | 3, 6 | 0.672 | 0.54 | 0.804 | 0.391 | 0.523 | 0.158 | 0.0682 | 0.415 | 449 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 21 | 3 | hsa-miR- | 2 | 4, 5 | 0.672 | 0.54 | 0.804 | 0.391 | 0.523 | 0.158 | 0 | 0.2 | 167.7 |
| | | 99b- | | | | | | | | | | | |
| | | 3p/hsa- | | | | | | | | | | | |
| | | miR-760 | | | | | | | | | | | |
| 21 | 6 | hsa-miR- | 2 | 7, 8 | 0.672 | 0.54 | 0.804 | 0.391 | 0.523 | 0.158 | 0.25 | 0.619 | 239.2 |
| | | 7-5p/hsa- | | | | | | | | | | | |
| | | miR- | | | | | | | | | | | |
| | | 4446-3p | | | | | | | | | | | |
| 22 | 1 | hsa-miR- | 5 | 2, 9 | 0.672 | 0.548 | 0.796 | 0.425 | 0.459 | 0.151 | 0.295 | 0.75 | 300.1 |
| | | 7-5p/hsa- | | | | | | | | | | | |
| | | miR-485- | | | | | | | | | | | |
| | | 5p | | | | | | | | | | | |
| 22 | 2 | hsa-miR- | 4 | 3, 6 | 0.672 | 0.548 | 0.796 | 0.425 | 0.459 | 0.151 | 0.21 | 0.562 | 147.7 |
| | | 221- | | | | | | | | | | | |
| | | 5p/hsa- | | | | | | | | | | | |
| | | miR-760 | | | | | | | | | | | |
| 22 | 3 | hsa-miR- | 2 | 4, 5 | 0.672 | 0.548 | 0.796 | 0.425 | 0.459 | 0.151 | 0.174 | 0.625 | 549.2 |
| | | 1260b/hsa | | | | | | | | | | | |
| | | -mir- | | | | | | | | | | | |
| | | 320c-2 | | | | | | | | | | | |
| 22 | 6 | hsa-miR- | 2 | 7, 8 | 0.672 | 0.548 | 0.796 | 0.425 | 0.459 | 0.151 | 0.154 | 0.842 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 23 | 1 | hsa-miR- | 6 | 2, 7 | 0.678 | 0.552 | 0.804 | 0.442 | 0.434 | 0.172 | 0.143 | 0.407 | 20.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 23 | 2 | hsa-miR- | 3 | 3, 6 | 0.678 | 0.552 | 0.804 | 0.442 | 0.434 | 0.172 | 0.037 | 0.5 | 156.0 |
| | | 378g/hsa- | | | | | | | | | | | |
| | | miR-485- | | | | | | | | | | | |
| | | 5p | | | | | | | | | | | |
| 23 | 3 | hsa-miR- | 2 | 4, 5 | 0.678 | 0.552 | 0.804 | 0.442 | 0.434 | 0.172 | 0 | 0.143 | 136.8 |
| | | 30c- | | | | | | | | | | | |
| | | 5p/hsa- | | | | | | | | | | | |
| | | miR-100- | | | | | | | | | | | |
| | | 5p | | | | | | | | | | | |
| 23 | 7 | hsa-miR- | 3 | 8, 11 | 0.678 | 0.552 | 0.804 | 0.442 | 0.434 | 0.172 | 0.353 | 0.909 | 155.6 |
| | | 501- | | | | | | | | | | | |
| | | 3p/hsa | | | | | | | | | | | |
| | | miR-484 | | | | | | | | | | | |
| 23 | 8 | hsa-miR- | 2 | 9, 10 | 0.678 | 0.552 | 0.804 | 0.442 | 0.434 | 0.172 | 0.25 | 0.654 | 158.7 |
| | | 221- | | | | | | | | | | | |
| | | 5p/hsa- | | | | | | | | | | | |
| | | miR-760 | | | | | | | | | | | |
| 24 | 1 | hsa-miR- | 6 | 2, 7 | 0.654 | 0.52 | 0.787 | 0.327 | 0.387 | 0.192 | 0.21 | 0.507 | 214.4 |
| | | 7-5p/hsa- | | | | | | | | | | | |
| | | miR-485- | | | | | | | | | | | |
| | | 5p | | | | | | | | | | | |
| 24 | 2 | hsa-miR- | 3 | 3, 6 | 0.654 | 0.52 | 0.787 | 0.327 | 0.387 | 0.192 | 0.151 | 0.75 | 298.7 |
| | | 221- | | | | | | | | | | | |
| | | 5p/hsa- | | | | | | | | | | | |
| | | miR-760 | | | | | | | | | | | |
| 24 | 3 | hsa-miR- | 2 | 4, 5 | 0.654 | 0.52 | 0.787 | 0.327 | 0.387 | 0.192 | 0 | 0.239 | 39.7 |
| | | 1306- | | | | | | | | | | | |
| | | 3p/hsa- | | | | | | | | | | | |
| | | miR- | | | | | | | | | | | |
| | | 148a-5p | | | | | | | | | | | |
| 24 | 7 | hsa-miR- | 3 | 8, 11 | 0.654 | 0.52 | 0.787 | 0.327 | 0.387 | 0.192 | 0.567 | 0 | 156.3 |
| | | 30c- | | | | | | | | | | | |
| | | 5p/hsa- | | | | | | | | | | | |
| | | miR-320b | | | | | | | | | | | |
| 24 | 8 | hsa-miR- | 2 | 9, 10 | 0.654 | 0.52 | 0.787 | 0.327 | 0.387 | 0.192 | 0.509 | 1 | 276.5 |
| | | 378e/hsa- | | | | | | | | | | | |
| | | miR-140- | | | | | | | | | | | |
| | | 5p | | | | | | | | | | | |
| 25 | 1 | hsa-miR- | 4 | 2, 3 | 0.624 | 0.507 | 0.742 | 0.293 | 0.190 | 0.297 | 0.857 | 0.319 | 93.8 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 25 | 3 | hsa-miR- | 3 | 4, 7 | 0.624 | 0.507 | 0.742 | 0.293 | 0.190 | 0.297 | 0.284 | 1 | 282.0 |
| | | 199a-3p- | | | | | | | | | | | |
| | | hsa-miR- | | | | | | | | | | | |
| | | 199b- | | | | | | | | | | | |
| | | 3p/hsa- | | | | | | | | | | | |
| | | miR-485- | | | | | | | | | | | |
| | | 5p | | | | | | | | | | | |
| 25 | 4 | hsa-miR- | 2 | 5, 6 | 0.624 | 0.507 | 0.742 | 0.293 | 0.190 | 0.297 | 0.0789 | 0.365 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 26 | 1 | hsa-miR- | 4 | 2, 7 | 0.661 | 0.552 | 0.77 | 0.462 | 0.172 | 0.121 | 0.285 | 0.778 | 318.6 |
| | | 7-5p/hsa- | | | | | | | | | | | |
| | | miR-485- | | | | | | | | | | | |
| | | 5p | | | | | | | | | | | |
| 26 | 2 | hsa-miR- | 3 | 3, 4 | 0.661 | 0.552 | 0.77 | 0.462 | 0.172 | 0.121 | 0.667 | 0.256 | 87.3 |
| | | 30c- | | | | | | | | | | | |
| | | 5p/hsa- | | | | | | | | | | | |
| | | miR- | | | | | | | | | | | |
| | | 125a-5p | | | | | | | | | | | |
| 26 | 4 | hsa-miR- | 2 | 5, 6 | 0.661 | 0.552 | 0.77 | 0.462 | 0.172 | 0.121 | 0.337 | 0.0312 | 260.7 |
| | | 1-3p/hsa- | | | | | | | | | | | |
| | | miR-345- | | | | | | | | | | | |
| | | 5p | | | | | | | | | | | |
| 27 | 1 | hsa-miR- | 5 | 2, 9 | 0.635 | 0.516 | 0.755 | 0.333 | 008.1 | 0.142 | 0.296 | 0.846 | 347.5 |
| | | 7-5p/hsa- | | | | | | | | | | | |
| | | miR-485- | | | | | | | | | | | |
| | | 5p | | | | | | | | | | | |
| 27 | 2 | hsa-miR- | 4 | 3, 6 | 0.635 | 0.516 | 0.755 | 0.333 | 0.108 | 0.142 | 0.373 | 0.0606 | 260.7 |
| | | 1-3p/hsa- | | | | | | | | | | | |
| | | miR-345- | | | | | | | | | | | |
| | | 5p | | | | | | | | | | | |
| 27 | 3 | hsa-miR- | 2 | 4, 5 | 0.635 | 0.516 | 0.755 | 0.333 | 0.108 | 0.142 | 0.282 | 0.824 | 226.7 |
| | | 99b- | | | | | | | | | | | |
| | | 3p/hsa- | | | | | | | | | | | |
| | | miR-483- | | | | | | | | | | | |
| | | 3p | | | | | | | | | | | |
| 27 | 6 | hsa-miR- | 2 | 7, 8 | 0.635 | 0.516 | 0.755 | 0.333 | 0.108 | 0.142 | 0.222 | 0 | 103.9 |
| | | 30c- | | | | | | | | | | | |
| | | 5p/hsa- | | | | | | | | | | | |
| | | miR-10b- | | | | | | | | | | | |
| | | 5p | | | | | | | | | | | |
| 28 | 1 | hsa-miR- | 6 | 2, 7 | 0.649 | 0.527 | 0.771 | 0.378 | 0.055 | 0.117 | 0.252 | 0.585 | 100.5 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 28 | 2 | hsa-miR- | 3 | 3, 6 | 0.649 | 0.527 | 0.771 | 0.378 | 0.055 | 0.117 | 0.17 | 0.846 | 330.8 |
| | | 7-5p/hsa- | | | | | | | | | | | |
| | | miR-485- | | | | | | | | | | | |
| | | 5p | | | | | | | | | | | |
| 28 | 3 | hsa-miR- | 2 | 4, 5 | 0.649 | 0.527 | 0.771 | 0.378 | 0.055 | 0.117 | 0.234 | 0.0333 | 199.4 |
| | | 433- | | | | | | | | | | | |
| | | 3p/hsa- | | | | | | | | | | | |
| | | miR-345- | | | | | | | | | | | |
| | | 5p | | | | | | | | | | | |
| 28 | 7 | hsa-miR- | 3 | 8, 9 | 0.649 | 0.527 | 0.771 | 0.378 | 0.055 | 0.117 | 0.882 | 0.375 | 147.6 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 28 | 9 | hsa-miR- | 2 | 10, 11 | 0.649 | 0.527 | 0.771 | 0.378 | 0.055 | 0.117 | 0.2 | 0.5 | 74.8 |
| | | 99b- | | | | | | | | | | | |
| | | 3p/hsa- | | | | | | | | | | | |
| | | miR- | | | | | | | | | | | |
| | | 148a-5p | | | | | | | | | | | |
| 29 | 1 | hsa-miR- | 5 | 2, 9 | 0.659 | 0.527 | 0.79 | 0.382 | −0.003 | 0.135 | 0.288 | 0.812 | 318.6 |
| | | 7-5p/hsa- | | | | | | | | | | | |
| | | miR-485- | | | | | | | | | | | |
| | | 5p | | | | | | | | | | | |
| 29 | 2 | hsa-miR- | 4 | 3, 6 | 0.659 | 0.527 | 0.79 | 0.382 | −0.003 | 0.135 | 0.0952 | 0.378 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 29 | 3 | hsa-miR- | 2 | 4, 5 | 0.659 | 0.527 | 0.79 | 0.382 | −0.003 | 0.135 | 0.154 | 0 | 223.6 |
| | | 1-3p/hsa- | | | | | | | | | | | |
| | | miR-345- | | | | | | | | | | | |
| | | 5p | | | | | | | | | | | |
| 29 | 6 | hsa-miR- | 2 | 7, 8 | 0.659 | 0.527 | 0.79 | 0.382 | −0.003 | 0.135 | 0.154 | 0.469 | 62.2 |
| | | 99b- | | | | | | | | | | | |
| | | 3p/hsa- | | | | | | | | | | | |
| | | miR- | | | | | | | | | | | |
| | | 518e-5p- | | | | | | | | | | | |
| | | hsa-miR- | | | | | | | | | | | |
| | | 519a-5p- | | | | | | | | | | | |
| | | hsa-miR- | | | | | | | | | | | |
| | | 519b-5p- | | | | | | | | | | | |
| | | hsa-miR- | | | | | | | | | | | |
| | | 519c-5p- | | | | | | | | | | | |
| | | hsa-miR- | | | | | | | | | | | |
| | | 522-5p- | | | | | | | | | | | |
| | | hsa-miR- | | | | | | | | | | | |
| | | 523-5p | | | | | | | | | | | |
| 30 | 1 | hsa-miR- | 5 | 2, 9 | 0.647 | 0.526 | 0.767 | 0.387 | −0.111 | 0.106 | 0.289 | 0.923 | 347.5 |
| | | 7-5p/hsa- | | | | | | | | | | | |
| | | miR-485- | | | | | | | | | | | |
| | | 5p | | | | | | | | | | | |
| 30 | 2 | hsa-miR- | 4 | 3, 6 | 0.647 | 0.526 | 0.767 | 0.387 | −0.111 | 0.106 | 0.0851 | 0.398 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 30 | 3 | hsa-miR- | 2 | 4, 5 | 0.647 | 0.526 | 0.767 | 0.387 | −0.111 | 0.106 | 0.0286 | 0.25 | 164.6 |
| | | 99b- | | | | | | | | | | | |
| | | 3p/hsa- | | | | | | | | | | | |
| | | miR-760 | | | | | | | | | | | |
| 30 | 6 | hsa-miR- | 2 | 7, 8 | 0.647 | 0.526 | 0.767 | 0.387 | −0.111 | 0.106 | 0.68 | 0.286 | 99.4 |
| | | 760/hsa- | | | | | | | | | | | |
| | | miR-345- | | | | | | | | | | | |
| | | 5p | | | | | | | | | | | |
| 31 | 1 | hsa-miR- | 8 | 2, 9 | 0.66 | 0.521 | 0.798 | 0.376 | −0.211 | 0.146 | 0.184 | 0.514 | 214.4 |
| | | 7-5p/hsa- | | | | | | | | | | | |
| | | miR-485- | | | | | | | | | | | |
| | | 5p | | | | | | | | | | | |
| 31 | 2 | hsa-miR- | 4 | 3, 6 | 0.66 | 0.521 | 0.798 | 0.376 | −0.211 | 0.146 | 0.0625 | 0.273 | 196.8 |
| | | 26b- | | | | | | | | | | | |
| | | 5p/hsa- | | | | | | | | | | | |
| | | miR-769- | | | | | | | | | | | |
| | | 5p | | | | | | | | | | | |
| 31 | 3 | hsa-miR- | 2 | 4, 5 | 0.66 | 0.521 | 0.798 | 0.376 | −0.211 | 0.146 | 0 | 0.125 | 53.8 |
| | | 99b- | | | | | | | | | | | |
| | | 3p/hsa- | | | | | | | | | | | |
| | | miR-760 | | | | | | | | | | | |
| 31 | 6 | hsa-miR- | 2 | 7, 8 | 0.66 | 0.521 | 0.798 | 0.376 | −0.211 | 0.146 | 0.189 | 0.714 | 267.6 |
| | | 99b- | | | | | | | | | | | |
| | | 3p/hsa- | | | | | | | | | | | |
| | | miR-144- | | | | | | | | | | | |
| | | 5p | | | | | | | | | | | |
| 31 | 9 | hsa-miR- | 4 | 10, 13 | 0.66 | 0.521 | 0.798 | 0.376 | −0.211 | 0.146 | 0.303 | 0.692 | 47.5 |
| | | 221- | | | | | | | | | | | |
| | | 5p/hsa- | | | | | | | | | | | |
| | | miR-197- | | | | | | | | | | | |
| | | 3p | | | | | | | | | | | |
| 31 | 10 | hsa-miR- | 2 | 11, 12 | 0.66 | 0.521 | 0.798 | 0.376 | −0.211 | 0.146 | 0.154 | 0.857 | 166.3 |
| | | 378e/hsa- | | | | | | | | | | | |
| | | miR-483- | | | | | | | | | | | |
| | | 3p | | | | | | | | | | | |
| 31 | 13 | hsa-miR- | 2 | 14, 15 | 0.66 | 0.521 | 0.798 | 0.376 | −0.211 | 0.146 | 0.429 | 0.84 | 80.4 |
| | | 194- | | | | | | | | | | | |
| | | 5p/hsa- | | | | | | | | | | | |
| | | miR-486- | | | | | | | | | | | |
| | | 3p | | | | | | | | | | | |
| 32 | 1 | hsa-miR- | 5 | 2, 5 | 0.651 | 0.518 | 0.784 | 0.367 | −0.243 | 0.132 | 0.156 | 0.427 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 32 | 2 | hsa-miR- | 2 | 3, 4 | 0.651 | 0.518 | 0.784 | 0.367 | −0.243 | 0.132 | 0.0526 | 0.714 | 156.0 |
| | | 378g/hsa- | | | | | | | | | | | |
| | | miR-485- | | | | | | | | | | | |
| | | 5p | | | | | | | | | | | |
| 32 | 5 | hsa-miR- | 3 | 6, 7 | 0.651 | 0.518 | 0.784 | 0.367 | −0.243 | 0.132 | 0 | 0.494 | −77.0 |
| | | 628- | | | | | | | | | | | |
| | | 3p/hsa- | | | | | | | | | | | |
| | | miR-19b- | | | | | | | | | | | |
| | | 3p | | | | | | | | | | | |
| 32 | 7 | hsa-miR- | 2 | 8, 9 | 0.651 | 0.518 | 0.784 | 0.367 | −0.243 | 0.132 | 0.369 | 0.833 | 146.1 |
| | | 221- | | | | | | | | | | | |
| | | 5p/hsa- | | | | | | | | | | | |
| | | miR-760 | | | | | | | | | | | |
| 33 | 1 | hsa-miR- | 4 | 2, 7 | 0.619 | 0.504 | 0.734 | 0.321 | −0.313 | 0.188 | 0.293 | 0.8 | 318.6 |
| | | 7-5p/hsa- | | | | | | | | | | | |
| | | miR-485- | | | | | | | | | | | |
| | | 5p | | | | | | | | | | | |
| 33 | 2 | hsa-miR- | 3 | 3, 6 | 0.619 | 0.504 | 0.734 | 0.321 | −0.313 | 0.188 | 0.244 | 0.9 | 652.0 |
| | | 1260b/hsa | | | | | | | | | | | |
| | | -mir- | | | | | | | | | | | |
| | | 320c-2 | | | | | | | | | | | |
| 33 | 3 | hsa-miR- | 2 | 4, 5 | 0.619 | 0.504 | 0.734 | 0.321 | −0.313 | 0.188 | 0.341 | 0 | 217.5 |
| | | 433- | | | | | | | | | | | |
| | | 3p/hsa- | | | | | | | | | | | |
| | | miR-345- | | | | | | | | | | | |
| | | 5p | | | | | | | | | | | |
| 34 | 1 | hsa-miR- | 5 | 2, 9 | 0.636 | 0.505 | 0.767 | 0.325 | −0.318 | 0.174 | 0.302 | 1 | 359.8 |
| | | 7-5p/hsa- | | | | | | | | | | | |
| | | miR-485- | | | | | | | | | | | |
| | | 5p | | | | | | | | | | | |
| 34 | 2 | hsa-miR- | 4 | 3, 6 | 0.636 | 0.505 | 0.767 | 0.325 | −0.318 | 0.174 | 0.237 | 0.667 | 147.6 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 34 | 3 | hsa-miR- | 2 | 4, 5 | 0.636 | 0.505 | 0.767 | 0.325 | −0.318 | 0.174 | 0.31 | 0.0588 | 260.7 |
| | | 1-3p/hsa- | | | | | | | | | | | |
| | | miR-345- | | | | | | | | | | | |
| | | 5p | | | | | | | | | | | |
| 34 | 6 | hsa-miR- | 2 | 7, 8 | 0.636 | 0.505 | 0.767 | 0.325 | −0.318 | 0.174 | 0.9 | 0.455 | 122.8 |
| | | 760/hsa- | | | | | | | | | | | |
| | | miR-345- | | | | | | | | | | | |
| | | 5p | | | | | | | | | | | |
| 35 | 1 | hsa-miR- | 6 | 2, 7 | 0.677 | 0.535 | 0.819 | 0.443 | −0.409 | 0.215 | 0.242 | 0.528 | 85.6 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 35 | 2 | hsa-miR- | 3 | 3, 6 | 0.677 | 0.535 | 0.819 | 0.443 | −0.409 | 0.215 | 0.193 | 0.857 | 330.8 |
| | | 7-5p/hsa- | | | | | | | | | | | |
| | | miR-485- | | | | | | | | | | | |
| | | 5p | | | | | | | | | | | |
| 35 | 3 | hsa-miR- | 2 | 4, 5 | 0.677 | 0.535 | 0.819 | 0.443 | −0.409 | 0.215 | 0.314 | 0.113 | 153.1 |
| | | 760/hsa- | | | | | | | | | | | |
| | | miR-345- | | | | | | | | | | | |
| | | 5p | | | | | | | | | | | |
| 35 | 7 | hsa-miR- | 3 | 8, 9 | 0.677 | 0.535 | 0.819 | 0.443 | −0.409 | 0.215 | 0.214 | 0.641 | 25.1 |
| | | 221- | | | | | | | | | | | |
| | | 5p/hsa- | | | | | | | | | | | |
| | | miR- | | | | | | | | | | | |
| | | 1908-5p | | | | | | | | | | | |
| 35 | 9 | hsa-miR- | 2 | 10, 11 | 0.677 | 0.535 | 0.819 | 0.443 | −0.409 | 0.215 | 0.889 | 0.567 | 130.3 |
| | | 30c- | | | | | | | | | | | |
| | | 5p/hsa- | | | | | | | | | | | |
| | | miR-7-5p | | | | | | | | | | | |
| 36 | 1 | hsa-miR- | 5 | 2, 9 | 0.629 | 0.509 | 0.748 | 0.347 | −0.414 | 0.126 | 0.312 | 1 | 413.5 |
| | | 7-5p/hsa- | | | | | | | | | | | |
| | | miR-485- | | | | | | | | | | | |
| | | 5p | | | | | | | | | | | |
| 36 | 2 | hsa-miR- | 4 | 3, 6 | 0.629 | 0.509 | 0.748 | 0.347 | −0.414 | 0.126 | 0.087 | 0.421 | 40.6 |
| | | 1306- | | | | | | | | | | | |
| | | 3p/hsa- | | | | | | | | | | | |
| | | miR- | | | | | | | | | | | |
| | | 1224-5p | | | | | | | | | | | |
| 36 | 3 | hsa-miR- | 2 | 4, 5 | 0.629 | 0.509 | 0.748 | 0.347 | −0.414 | 0.126 | 0 | 0.364 | 164.6 |
| | | 99b- | | | | | | | | | | | |
| | | 3p/hsa- | | | | | | | | | | | |
| | | miR-760 | | | | | | | | | | | |
| 36 | 6 | hsa-miR- | 2 | 7, 8 | 0.629 | 0.509 | 0.748 | 0.347 | −0.414 | 0.126 | 0.8 | 0.376 | −285.5 |
| | | 543/hsa- | | | | | | | | | | | |
| | | miR-345- | | | | | | | | | | | |
| | | 5p | | | | | | | | | | | |
| 37 | 1 | hsa-miR- | 4 | 2, 7 | 0.661 | 0.553 | 0.77 | 0.515 | −0.478 | 0.124 | 0.285 | 0.778 | 330.8 |
| | | 7-5p/hsa- | | | | | | | | | | | |
| | | miR-485- | | | | | | | | | | | |
| | | 5p | | | | | | | | | | | |
| 37 | 2 | hsa-miR- | 3 | 3, 6 | 0.661 | 0.553 | 0.77 | 0.515 | −0.478 | 0.124 | 0.239 | 0.588 | 162.9 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 37 | 3 | hsa-miR- | 2 | 4, 5 | 0.661 | 0.553 | 0.77 | 0.515 | −0.478 | 0.124 | 0.714 | 0.208 | 100.9 |
| | | 30c- | | | | | | | | | | | |
| | | 5p/hsa- | | | | | | | | | | | |
| | | miR-7-5p | | | | | | | | | | | |
| 38 | 1 | hsa-miR- | 4 | 2, 7 | 0.635 | 0.507 | 0.763 | 0.345 | −0.484 | 0.133 | 0.309 | 0.889 | 396.4 |
| | | 7-5p/hsa- | | | | | | | | | | | |
| | | miR-485- | | | | | | | | | | | |
| | | 5p | | | | | | | | | | | |
| 38 | 2 | hsa-miR- | 3 | 3, 6 | 0.635 | 0.507 | 0.763 | 0.345 | −0.484 | 0.133 | 0.26 | 0.833 | 172.2 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 38 | 3 | hsa-miR- | 2 | 4, 5 | 0.635 | 0.507 | 0.763 | 0.345 | −0.484 | 0.133 | 0.323 | 0.0882 | 260.7 |
| | | 1-3p/hsa- | | | | | | | | | | | |
| | | miR-345- | | | | | | | | | | | |
| | | 5p | | | | | | | | | | | |
| 39 | 1 | hsa-miR- | 5 | 2, 9 | 0.637 | 0.514 | 0.761 | 0.376 | −0.550 | 0.105 | 0.304 | 0.9 | 295.2 |
| | | 98- | | | | | | | | | | | |
| | | 5p/hsa- | | | | | | | | | | | |
| | | miR-485- | | | | | | | | | | | |
| | | 5p | | | | | | | | | | | |
| 39 | 2 | hsa-miR- | 4 | 3, 6 | 0.637 | 0.514 | 0.761 | 0.376 | −0.550 | 0.105 | 0.233 | 0.682 | 162.9 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 39 | 3 | hsa-miR- | 2 | 4, 5 | 0.637 | 0.514 | 0.761 | 0.376 | −0.550 | 0.105 | 0.304 | 0.0811 | 118.4 |
| | | 30c- | | | | | | | | | | | |
| | | 5p/hsa- | | | | | | | | | | | |
| | | miR-10b- | | | | | | | | | | | |
| | | 5p | | | | | | | | | | | |
| 39 | 6 | hsa-miR- | 2 | 7, 8 | 0.637 | 0.514 | 0.761 | 0.376 | −0.550 | 0.105 | 0.909 | 0.455 | 100.7 |
| | | 433- | | | | | | | | | | | |
| | | 3p/hsa- | | | | | | | | | | | |
| | | miR-345- | | | | | | | | | | | |
| | | 5p | | | | | | | | | | | |
| 40 | 1 | hsa-miR- | 5 | 2, 9 | 0.642 | 0.511 | 0.773 | 0.365 | −0.555 | 0.120 | 0.288 | 0.812 | 330.8 |
| | | 7-5p/hsa- | | | | | | | | | | | |
| | | miR-485- | | | | | | | | | | | |
| | | 5p | | | | | | | | | | | |
| 40 | 2 | hsa-miR- | 4 | 3, 6 | 0.642 | 0.511 | 0.773 | 0.365 | −0.555 | 0.120 | 0.0943 | 0.418 | 54.6 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 40 | 3 | hsa-miR- | 2 | 4, 5 | 0.642 | 0.511 | 0.773 | 0.365 | −0.555 | 0.120 | 0.135 | 0 | 245.4 |
| | | 1-3p/hsa- | | | | | | | | | | | |
| | | miR-345- | | | | | | | | | | | |
| | | 5p | | | | | | | | | | | |
| 40 | 6 | hsa-miR- | 2 | 7, 8 | 0.642 | 0.511 | 0.773 | 0.365 | −0.555 | 0.120 | 0.286 | 0.739 | 128.5 |
| | | 628- | | | | | | | | | | | |
| | | 3p/hsa- | | | | | | | | | | | |
| | | miR-19b- | | | | | | | | | | | |
| | | 3p | | | | | | | | | | | |
| 41 | 1 | hsa-miR- | 5 | 2, 9 | 0.654 | 0.521 | 0.788 | 0.403 | −0.566 | 0.113 | 0.284 | 0.929 | 306.5 |
| | | 7-5p/hsa- | | | | | | | | | | | |
| | | miR-485- | | | | | | | | | | | |
| | | 5p | | | | | | | | | | | |
| 41 | 2 | hsa-miR- | 4 | 3, 6 | 0.654 | 0.521 | 0.788 | 0.403 | −0.566 | 0.113 | 0.0513 | 0.379 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 41 | 3 | hsa-miR- | 2 | 4, 5 | 0.654 | 0.521 | 0.788 | 0.403 | −0.566 | 0.113 | 0 | 0.286 | 221.2 |
| | | 99b- | | | | | | | | | | | |
| | | 3p/hsa- | | | | | | | | | | | |
| | | miR-760 | | | | | | | | | | | |
| 41 | 6 | hsa-miR- | 2 | 7, 8 | 0.654 | 0.521 | 0.788 | 0.403 | −0.566 | 0.113 | 0.208 | 0.595 | 121.5 |
| | | 323b- | | | | | | | | | | | |
| | | 3p/hsa- | | | | | | | | | | | |
| | | mir-320c- | | | | | | | | | | | |
| | | 2 | | | | | | | | | | | |
| 42 | 1 | hsa-miR- | 6 | 2, 9 | 0.635 | 0.501 | 0.77 | 0.330 | −0.589 | 0.149 | 0.276 | 0.762 | 298.0 |
| | | 7-5p/hsa- | | | | | | | | | | | |
| | | miR-485- | | | | | | | | | | | |
| | | 5p | | | | | | | | | | | |
| 42 | 2 | hsa-miR- | 4 | 3, 6 | 0.635 | 0.501 | 0.77 | 0.330 | −0.589 | 0.149 | 0.181 | 0.545 | 147.7 |
| | | 221- | | | | | | | | | | | |
| | | 5p/hsa- | | | | | | | | | | | |
| | | miR-760 | | | | | | | | | | | |
| 42 | 3 | hsa-miR- | 2 | 4, 5 | 0.635 | 0.501 | 0.77 | 0.330 | −0.589 | 0.149 | 0.0714 | 0.342 | 105.1 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 42 | 6 | hsa-miR- | 2 | 7, 8 | 0.635 | 0.501 | 0.77 | 0.330 | −0.589 | 0.149 | 0.1 | 0.739 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 42 | 9 | hsa-miR- | 2 | 10, 11 | 0.635 | 0.501 | 0.77 | 0.330 | −0.589 | 0.149 | 0.286 | 1 | 95.0 |
| | | 766- | | | | | | | | | | | |
| | | 3p/hsa- | | | | | | | | | | | |
| | | miR- | | | | | | | | | | | |
| | | 148a-5p | | | | | | | | | | | |
| 43 | 1 | hsa-miR- | 5 | 2, 9 | 0.646 | 0.514 | 0.778 | 0.388 | −0.709 | 0.101 | 0.294 | 0.917 | 359.8 |
| | | 7-5p/hsa- | | | | | | | | | | | |
| | | miR-485- | | | | | | | | | | | |
| | | 5p | | | | | | | | | | | |
| 43 | 2 | hsa-miR- | 4 | 3, 6 | 0.646 | 0.514 | 0.778 | 0.388 | −0.709 | 0.101 | 0.111 | 0.385 | 46.5 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 43 | 3 | hsa-miR- | 2 | 4, 5 | 0.646 | 0.514 | 0.778 | 0.388 | −0.709 | 0.101 | 0.0278 | 0.444 | 230.8 |
| | | 99b- | | | | | | | | | | | |
| | | 3p/hsa- | | | | | | | | | | | |
| | | miR-760 | | | | | | | | | | | |
| 43 | 6 | hsa-miR- | 2 | 7, 8 | 0.646 | 0.514 | 0.778 | 0.388 | −0.709 | 0.101 | 0.316 | 0.833 | 186.8 |
| | | 7-5p/hsa- | | | | | | | | | | | |
| | | let-7c-5p | | | | | | | | | | | |
| 44 | 1 | hsa-miR- | 5 | 2, 3 | 0.638 | 0.507 | 0.769 | 0.362 | −0.716 | 0.119 | 1 | 0.312 | 98.2 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 44 | 3 | hsa-miR- | 4 | 4, 7 | 0.638 | 0.507 | 0.769 | 0.362 | −0.716 | 0.119 | 0.075 | 0.406 | 172.5 |
| | | 7-5p/hsa- | | | | | | | | | | | |
| | | miR-485- | | | | | | | | | | | |
| | | 5p | | | | | | | | | | | |
| 44 | 4 | hsa-miR- | 2 | 5, 6 | 0.638 | 0.507 | 0.769 | 0.362 | −0.716 | 0.119 | 0 | 0.273 | 239.7 |
| | | 1260b/hsa | | | | | | | | | | | |
| | | -mir- | | | | | | | | | | | |
| | | 320c-2 | | | | | | | | | | | |
| 44 | 7 | hsa-miR- | 2 | 8, 9 | 0.638 | 0.507 | 0.769 | 0.362 | −0.716 | 0.119 | 0.579 | 0.182 | 159.1 |
| | | 433- | | | | | | | | | | | |
| | | 3p/hsa- | | | | | | | | | | | |
| | | miR-345- | | | | | | | | | | | |
| | | 5p | | | | | | | | | | | |
| 45 | 1 | hsa-miR- | 7 | 2, 7 | 0.703 | 0.564 | 0.843 | 0.576 | −0.749 | 0.415 | 0.116 | 0.438 | 172.5 |
| | | 7-5p/hsa- | | | | | | | | | | | |
| | | miR-485- | | | | | | | | | | | |
| | | 5p | | | | | | | | | | | |
| 45 | 2 | hsa-miR- | 3 | 3, 6 | 0.703 | 0.564 | 0.843 | 0.576 | −0.749 | 0.415 | 0.0286 | 0.5 | 76.0 |
| | | 23b- | | | | | | | | | | | |
| | | 5p/hsa- | | | | | | | | | | | |
| | | miR-2110 | | | | | | | | | | | |
| 45 | 3 | hsa-miR- | 2 | 4, 5 | 0.703 | 0.564 | 0.843 | 0.576 | −0.749 | 0.415 | 0 | 0.143 | 134.5 |
| | | 221- | | | | | | | | | | | |
| | | 5p/hsa- | | | | | | | | | | | |
| | | miR-760 | | | | | | | | | | | |
| 45 | 7 | hsa-miR- | 4 | 8, 11 | 0.703 | 0.564 | 0.843 | 0.576 | −0.749 | 0.415 | 0.278 | 0.608 | 101.9 |
| | | 323b- | | | | | | | | | | | |
| | | 3p/hsa- | | | | | | | | | | | |
| | | mir-320c- | | | | | | | | | | | |
| | | 2 | | | | | | | | | | | |
| 45 | 8 | hsa-miR- | 2 | 9, 10 | 0.703 | 0.564 | 0.843 | 0.576 | −0.749 | 0.415 | 0.158 | 0.562 | 127.0 |
| | | 20a- | | | | | | | | | | | |
| | | 5p/hsa- | | | | | | | | | | | |
| | | miR-486- | | | | | | | | | | | |
| | | 3p | | | | | | | | | | | |
| 45 | 11 | hsa-miR- | 2 | 12, 13 | 0.703 | 0.564 | 0.843 | 0.576 | −0.749 | 0.415 | 0.484 | 0.8 | 198.5 |
| | | 511- | | | | | | | | | | | |
| | | 5p/hsa- | | | | | | | | | | | |
| | | miR- | | | | | | | | | | | |
| | | 148a-5p | | | | | | | | | | | |
| 46 | 1 | hsa-miR- | 6 | 2, 7 | 0.657 | 0.526 | 0.788 | 0.441 | −0.826 | 0.113 | 0.207 | 0.515 | 216.4 |
| | | 7-5p/hsa- | | | | | | | | | | | |
| | | miR-485- | | | | | | | | | | | |
| | | 5p | | | | | | | | | | | |
| 46 | 2 | hsa-miR- | 3 | 3, 6 | 0.657 | 0.526 | 0.788 | 0.441 | −0.826 | 0.113 | 0.149 | 0.75 | 298.7 |
| | | 221- | | | | | | | | | | | |
| | | 5p/hsa- | | | | | | | | | | | |
| | | miR-760 | | | | | | | | | | | |
| 46 | 3 | hsa-miR- | 2 | 4, 5 | 0.657 | 0.526 | 0.788 | 0.441 | −0.826 | 0.113 | 0 | 0.314 | 56.4 |
| | | 15b- | | | | | | | | | | | |
| | | 5p/hsa- | | | | | | | | | | | |
| | | miR-197- | | | | | | | | | | | |
| | | 3p | | | | | | | | | | | |
| 46 | 7 | hsa-miR- | 3 | 8, 9 | 0.657 | 0.526 | 0.788 | 0.441 | −0.826 | 0.113 | 0.143 | 0.615 | 67.4 |
| | | 194- | | | | | | | | | | | |
| | | 5p/hsa- | | | | | | | | | | | |
| | | miR-486- | | | | | | | | | | | |
| | | 3p | | | | | | | | | | | |
| 46 | 9 | hsa-miR- | 2 | 10, 11 | 0.657 | 0.526 | 0.788 | 0.441 | −0.826 | 0.113 | 0.423 | 0.808 | 75.0 |
| | | 1306- | | | | | | | | | | | |
| | | 3p/hsa- | | | | | | | | | | | |
| | | miR-335- | | | | | | | | | | | |
| | | 3p | | | | | | | | | | | |
| 47 | 1 | hsa-miR- | 5 | 2, 9 | 0.672 | 0.535 | 0.808 | 0.490 | −1.035 | 0.145 | 0.304 | 0.9 | 295.2 |
| | | 98- | | | | | | | | | | | |
| | | 5p/hsa- | | | | | | | | | | | |
| | | miR-485- | | | | | | | | | | | |
| | | 5p | | | | | | | | | | | |
| 47 | 2 | hsa-miR- | 4 | 3, 6 | 0.672 | 0.535 | 0.808 | 0.490 | −1.035 | 0.145 | 0.087 | 0.413 | 44.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 47 | 3 | hsa-miR- | 2 | 4, 5 | 0.672 | 0.535 | 0.808 | 0.490 | −1.035 | 0.145 | 0.0256 | 0.429 | 115.0 |
| | | 99b- | | | | | | | | | | | |
| | | 3p/hsa- | | | | | | | | | | | |
| | | miR-485- | | | | | | | | | | | |
| | | 5p | | | | | | | | | | | |
| 47 | 6 | hsa-miR- | 2 | 7, 8 | 0.672 | 0.535 | 0.808 | 0.490 | −1.035 | 0.145 | 0.293 | 0.618 | 56.2 |
| | | 221- | | | | | | | | | | | |
| | | 5p/hsa- | | | | | | | | | | | |
| | | miR-197- | | | | | | | | | | | |
| | | 3p | | | | | | | | | | | |
| 48 | 1 | hsa-miR- | 5 | 2, 9 | 0.676 | 0.541 | 0.812 | 0.516 | −1.084 | 0.163 | 0.395 | 0.138 | 181.3 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 48 | 2 | hsa-miR- | 4 | 3, 6 | 0.676 | 0.541 | 0.812 | 0.516 | −1.084 | 0.163 | 0.303 | 0.667 | 76.0 |
| | | 221- | | | | | | | | | | | |
| | | 5p/hsa- | | | | | | | | | | | |
| | | miR-197- | | | | | | | | | | | |
| | | 3p | | | | | | | | | | | |
| 48 | 3 | hsa-miR- | 2 | 4, 5 | 0.676 | 0.541 | 0.812 | 0.516 | −1.084 | 0.163 | 0.211 | 0.667 | 105.1 |
| | | 1306- | | | | | | | | | | | |
| | | 3p/hsa- | | | | | | | | | | | |
| | | miR-150- | | | | | | | | | | | |
| | | 3p | | | | | | | | | | | |
| 48 | 6 | hsa-miR- | 2 | 7, 8 | 0.676 | 0.541 | 0.812 | 0.516 | −1.084 | 0.163 | 0.4 | 0.8 | 15.2 |
| | | 15b- | | | | | | | | | | | |
| | | 5p/hsa- | | | | | | | | | | | |
| | | miR-197- | | | | | | | | | | | |
| | | 3p | | | | | | | | | | | |
| 49 | 1 | hsa-miR- | 4 | 2, 7 | 0.652 | 0.528 | 0.775 | 0.479 | −1.234 | 0.097 | 0.301 | 0.833 | 330.8 |
| | | 7-5p/hsa- | | | | | | | | | | | |
| | | miR-485- | | | | | | | | | | | |
| | | 5p | | | | | | | | | | | |
| 49 | 2 | hsa-miR- | 3 | 3, 6 | 0.652 | 0.528 | 0.775 | 0.479 | −1.234 | 0.097 | 0.26 | 0.692 | 172.2 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 49 | 3 | hsa-miR- | 2 | 4, 5 | 0.652 | 0.528 | 0.775 | 0.479 | −1.234 | 0.097 | 0.7 | 0.221 | 79.5 |
| | | 30c- | | | | | | | | | | | |
| | | 5p/hsa- | | | | | | | | | | | |
| | | miR-21- | | | | | | | | | | | |
| | | 5p | | | | | | | | | | | |
| 50 | 1 | hsa-miR- | 7 | 2, 7 | 0.682 | 0.546 | 0.818 | 0.546 | −1.238 | 0.216 | 0.0976 | 0.439 | 172.5 |
| | | 7-5p/hsa- | | | | | | | | | | | |
| | | miR-485- | | | | | | | | | | | |
| | | 5p | | | | | | | | | | | |
| 50 | 2 | hsa-miR- | 3 | 3, 6 | 0.682 | 0.546 | 0.818 | 0.546 | −1.238 | 0.216 | 0.0294 | 0.429 | 130.4 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 50 | 3 | hsa-miR- | 2 | 4, 5 | 0.682 | 0.546 | 0.818 | 0.546 | −1.238 | 0.216 | 0 | 0.143 | 142.2 |
| | | 30c- | | | | | | | | | | | |
| | | 5p/hsa- | | | | | | | | | | | |
| | | miR-100- | | | | | | | | | | | |
| | | 5p | | | | | | | | | | | |
| 50 | 7 | hsa-miR- | 4 | 8, 11 | 0.682 | 0.546 | 0.818 | 0.546 | −1.238 | 0.216 | 0.574 | 0.261 | 153.9 |
| | | 30c- | | | | | | | | | | | |
| | | 5p/hsa- | | | | | | | | | | | |
| | | miR-425- | | | | | | | | | | | |
| | | 5p | | | | | | | | | | | |
| 50 | 8 | hsa-miR- | 2 | 9, 10 | 0.682 | 0.546 | 0.818 | 0.546 | −1.238 | 0.216 | 0.465 | 0.833 | 276.5 |
| | | 511- | | | | | | | | | | | |
| | | 5p/hsa- | | | | | | | | | | | |
| | | miR- | | | | | | | | | | | |
| | | 148a-5p | | | | | | | | | | | |
| 50 | 11 | hsa-miR- | 2 | 12, 13 | 0.682 | 0.546 | 0.818 | 0.546 | −1.238 | 0.216 | 0.205 | 0.571 | 211.0 |
| | | 99b- | | | | | | | | | | | |
| | | 3p/hsa- | | | | | | | | | | | |
| | | miR-483- | | | | | | | | | | | |
| | | 3p | | | | | | | | | | | |
| 51 | 1 | hsa-miR- | 6 | 2, 7 | 0.65 | 0.503 | 0.798 | 0.388 | −1.257 | 0.116 | 0.416 | 0.114 | 173.7 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 51 | 2 | hsa-miR- | 3 | 3, 6 | 0.65 | 0.503 | 0.798 | 0.388 | −1.257 | 0.116 | 0.337 | 0.833 | 328.4 |
| | | 7-5p/hsa- | | | | | | | | | | | |
| | | miR- | | | | | | | | | | | |
| | | 4446-3p | | | | | | | | | | | |
| 51 | 3 | hsa-miR- | 2 | 4, 5 | 0.65 | 0.503 | 0.798 | 0.388 | −1.257 | 0.116 | 0.174 | 0.49 | 45.8 |
| | | 1260b/hsa | | | | | | | | | | | |
| | | -mir- | | | | | | | | | | | |
| | | 320c-2 | | | | | | | | | | | |
| 51 | 7 | hsa-miR- | 3 | 8, 9 | 0.65 | 0.503 | 0.798 | 0.388 | −1.257 | 0.116 | 0.3 | 0.04 | −156.0 |
| | | 1260b/hsa | | | | | | | | | | | |
| | | -mir- | | | | | | | | | | | |
| | | 320c-2 | | | | | | | | | | | |
| 51 | 9 | hsa-miR- | 2 | 10, 11 | 0.65 | 0.503 | 0.798 | 0.388 | −1.257 | 0.116 | 0 | 0.143 | 205.3 |
| | | 1260b/hsa | | | | | | | | | | | |
| | | -mir- | | | | | | | | | | | |
| | | 320c-2 | | | | | | | | | | | |
| 52 | 1 | hsa-miR- | 4 | 2, 7 | 0.645 | 0.519 | 0.771 | 0.456 | −1.366 | 0.083 | 0.307 | 0.818 | 359.8 |
| | | 7-5p/hsa- | | | | | | | | | | | |
| | | miR-485- | | | | | | | | | | | |
| | | 5p | | | | | | | | | | | |
| 52 | 2 | hsa-miR- | 3 | 3, 6 | 0.645 | 0.519 | 0.771 | 0.456 | −1.366 | 0.083 | 0.25 | 0.706 | 162.9 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 52 | 3 | hsa-miR- | 2 | 4, 5 | 0.645 | 0.519 | 0.771 | 0.456 | −1.366 | 0.083 | 0.75 | 0.214 | 79.2 |
| | | 30c- | | | | | | | | | | | |
| | | 5p/hsa- | | | | | | | | | | | |
| | | let-7g-5p | | | | | | | | | | | |
| 53 | 1 | hsa-miR- | 5 | 2, 9 | 0.644 | 0.518 | 0.77 | 0.454 | −1.393 | 0.081 | 0.277 | 0.833 | 330.8 |
| | | 7-5p/hsa- | | | | | | | | | | | |
| | | miR-485- | | | | | | | | | | | |
| | | 5p | | | | | | | | | | | |
| 53 | 2 | hsa-miR- | 4 | 3, 6 | 0.644 | 0.518 | 0.77 | 0.454 | −1.393 | 0.081 | 0.0652 | 0.393 | 39.9 |
| | | 1306- | | | | | | | | | | | |
| | | 3p/hsa- | | | | | | | | | | | |
| | | miR- | | | | | | | | | | | |
| | | 1224-5p | | | | | | | | | | | |
| 53 | 3 | hsa-miR- | 2 | 4, 5 | 0.644 | 0.518 | 0.77 | 0.454 | −1.393 | 0.081 | 0 | 0.429 | 188.4 |
| | | 142- | | | | | | | | | | | |
| | | 5p/hsa- | | | | | | | | | | | |
| | | miR-485- | | | | | | | | | | | |
| | | 5p | | | | | | | | | | | |
| 53 | 6 | hsa-miR- | 2 | 7, 8 | 0.644 | 0.518 | 0.77 | 0.454 | −1.393 | 0.081 | 0.274 | 0.727 | 75.8 |
| | | 221- | | | | | | | | | | | |
| | | 5p/hsa- | | | | | | | | | | | |
| | | miR-197- | | | | | | | | | | | |
| | | 3p | | | | | | | | | | | |
| 54 | 1 | hsa-miR- | 4 | 2, 7 | 0.641 | 0.524 | 0.757 | 0.483 | −1.477 | 0.078 | 0.291 | 0.857 | 276.0 |
| | | 98- | | | | | | | | | | | |
| | | 5p/hsa- | | | | | | | | | | | |
| | | miR-485- | | | | | | | | | | | |
| | | 5p | | | | | | | | | | | |
| 54 | 2 | hsa-miR- | 3 | 3, 6 | 0.641 | 0.524 | 0.757 | 0.483 | −1.477 | 0.078 | 0.25 | 0.643 | 170.1 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 54 | 3 | hsa-miR- | 2 | 4, 5 | 0.641 | 0.524 | 0.757 | 0.483 | −1.477 | 0.078 | 0.714 | 0.221 | 103.3 |
| | | 30c- | | | | | | | | | | | |
| | | 5p/hsa- | | | | | | | | | | | |
| | | miR-625- | | | | | | | | | | | |
| | | 3p | | | | | | | | | | | |
| 55 | 1 | hsa-miR- | 6 | 2, 7 | 0.658 | 0.521 | 0.795 | 0.489 | −1.703 | 0.107 | 0.104 | 0.46 | 172.5 |
| | | 7-5p/hsa- | | | | | | | | | | | |
| | | miR-485- | | | | | | | | | | | |
| | | 5p | | | | | | | | | | | |
| 55 | 2 | hsa-miR- | 3 | 3, 6 | 0.658 | 0.521 | 0.795 | 0.489 | −1.703 | 0.107 | 0.0244 | 0.571 | 82.4 |
| | | 23b- | | | | | | | | | | | |
| | | 5p/hsa- | | | | | | | | | | | |
| | | miR-2110 | | | | | | | | | | | |
| 55 | 3 | hsa-miR- | 2 | 4, 5 | 0.658 | 0.521 | 0.795 | 0.489 | −1.703 | 0.107 | 0.143 | 0 | 100.7 |
| | | 30c- | | | | | | | | | | | |
| | | 5p/hsa- | | | | | | | | | | | |
| | | miR- | | | | | | | | | | | |
| | | 125a-5p | | | | | | | | | | | |
| 55 | 7 | hsa-miR- | 3 | 8, 11 | 0.658 | 0.521 | 0.795 | 0.489 | −1.703 | 0.107 | 0.529 | 0 | 473.0 |
| | | 760/hsa- | | | | | | | | | | | |
| | | miR-345- | | | | | | | | | | | |
| | | 5p | | | | | | | | | | | |
| 55 | 8 | hsa-miR- | 2 | 9, 10 | 0.658 | 0.521 | 0.795 | 0.489 | −1.703 | 0.107 | 0.421 | 0.733 | 121.4 |
| | | 99b- | | | | | | | | | | | |
| | | 3p/hsa- | | | | | | | | | | | |
| | | miR-150- | | | | | | | | | | | |
| | | 3p | | | | | | | | | | | |
| 56 | 1 | hsa-miR- | 6 | 2, 5 | 0.644 | 0.505 | 0.783 | 0.456 | −2.055 | 0.079 | 0.0833 | 0.429 | 31.9 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 56 | 2 | hsa-miR- | 2 | 3, 4 | 0.644 | 0.505 | 0.783 | 0.456 | −2.055 | 0.079 | 0 | 0.429 | 154.9 |
| | | 378g/hsa- | | | | | | | | | | | |
| | | miR-485- | | | | | | | | | | | |
| | | 5p | | | | | | | | | | | |
| 56 | 5 | hsa-miR- | 4 | 6, 9 | 0.644 | 0.505 | 0.783 | 0.456 | −2.055 | 0.079 | 0.571 | 0.343 | 144.8 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 56 | 6 | hsa-miR- | 2 | 7, 8 | 0.644 | 0.505 | 0.783 | 0.456 | −2.055 | 0.079 | 0.333 | 0.81 | 98.8 |
| | | 766- | | | | | | | | | | | |
| | | 3p/hsa- | | | | | | | | | | | |
| | | miR- | | | | | | | | | | | |
| | | 148a-5p | | | | | | | | | | | |
| 56 | 9 | hsa-miR- | 2 | 10, 11 | 0.644 | 0.505 | 0.783 | 0.456 | −2.055 | 0.079 | 0.15 | 0.42 | 55.6 |
| | | 99b- | | | | | | | | | | | |
| | | 3p/hsa- | | | | | | | | | | | |
| | | miR- | | | | | | | | | | | |
| | | 518e-5p- | | | | | | | | | | | |
| | | hsa-miR- | | | | | | | | | | | |
| | | 519a-5p- | | | | | | | | | | | |
| | | hsa-miR- | | | | | | | | | | | |
| | | 519b-5p- | | | | | | | | | | | |
| | | hsa-miR- | | | | | | | | | | | |
| | | 519c-5p- | | | | | | | | | | | |
| | | hsa-miR- | | | | | | | | | | | |
| | | 522-5p- | | | | | | | | | | | |
| | | hsa-miR- | | | | | | | | | | | |
| | | 523-5p | | | | | | | | | | | |
| 57 | 1 | hsa-miR- | 5 | 2, 9 | 0.641 | 0.503 | 0.78 | 0.456 | −2.147 | 0.077 | 0.285 | 0.778 | 300.1 |
| | | 7-5p/hsa- | | | | | | | | | | | |
| | | miR-485- | | | | | | | | | | | |
| | | 5p | | | | | | | | | | | |
| 57 | 2 | hsa-miR- | 4 | 3, 6 | 0.641 | 0.503 | 0.78 | 0.456 | −2.147 | 0.077 | 0.202 | 0.615 | 147.4 |
| | | 221- | | | | | | | | | | | |
| | | 5p/hsa- | | | | | | | | | | | |
| | | miR-760 | | | | | | | | | | | |
| 57 | 3 | hsa-miR- | 2 | 4, 5 | 0.641 | 0.503 | 0.78 | 0.456 | −2.147 | 0.077 | 0.111 | 0.341 | 104.4 |
| | | 99b- | | | | | | | | | | | |
| | | 3p/hsa- | | | | | | | | | | | |
| | | miR-340- | | | | | | | | | | | |
| | | 5p | | | | | | | | | | | |
| 57 | 6 | hsa-miR- | 2 | 7, 8 | 0.641 | 0.503 | 0.78 | 0.456 | −2.147 | 0.077 | 0.25 | 0.778 | 4.1 |
| | | 1306- | | | | | | | | | | | |
| | | 3p/hsa- | | | | | | | | | | | |
| | | miR-150- | | | | | | | | | | | |
| | | 3p | | | | | | | | | | | |
| 58 | 1 | hsa-miR- | 4 | 2, 7 | 0.638 | 0.506 | 0.77 | 0.476 | −2.264 | 0.071 | 0.294 | 0.917 | 347.5 |
| | | 7-5p/hsa- | | | | | | | | | | | |
| | | miR-485- | | | | | | | | | | | |
| | | 5p | | | | | | | | | | | |
| 58 | 2 | hsa-miR- | 3 | 3, 6 | 0.638 | 0.506 | 0.77 | 0.476 | −2.264 | 0.071 | 0.24 | 0.733 | 172.2 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 58 | 3 | hsa-miR- | 2 | 4, 5 | 0.638 | 0.506 | 0.77 | 0.476 | −2.264 | 0.071 | 0.143 | 0.459 | 265.3 |
| | | 323b- | | | | | | | | | | | |
| | | 3p/hsa- | | | | | | | | | | | |
| | | mir-320c- | | | | | | | | | | | |
| | | 2 | | | | | | | | | | | |
| 59 | 1 | hsa-miR- | 7 | 2, 7 | 0.644 | 0.504 | 0.785 | 0.483 | −2.453 | 0.080 | 0.189 | 0.432 | 46.5 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 59 | 2 | hsa-miR- | 3 | 3, 6 | 0.644 | 0.504 | 0.785 | 0.483 | −2.453 | 0.080 | 0.0526 | 0.533 | 158.9 |
| | | 378g/hsa- | | | | | | | | | | | |
| | | miR-485- | | | | | | | | | | | |
| | | 5p | | | | | | | | | | | |
| 59 | 3 | hsa-miR- | 2 | 4, 5 | 0.644 | 0.504 | 0.785 | 0.483 | −2.453 | 0.080 | 0 | 0.182 | 164.6 |
| | | 99b- | | | | | | | | | | | |
| | | 3p/hsa- | | | | | | | | | | | |
| | | miR-760 | | | | | | | | | | | |
| 59 | 7 | hsa-miR- | 4 | 8, 11 | 0.644 | 0.504 | 0.785 | 0.483 | −2.453 | 0.080 | 0.305 | 0.639 | 98.1 |
| | | 221- | | | | | | | | | | | |
| | | 5p/hsa- | | | | | | | | | | | |
| | | miR-760 | | | | | | | | | | | |
| 59 | 8 | hsa-miR- | 2 | 9, 10 | 0.644 | 0.504 | 0.785 | 0.483 | −2.453 | 0.080 | 0.378 | 0.0714 | 181.3 |
| | | 30c- | | | | | | | | | | | |
| | | 5p/hsa- | | | | | | | | | | | |
| | | miR-27b- | | | | | | | | | | | |
| | | 3p | | | | | | | | | | | |
| 59 | 11 | hsa-miR- | 2 | 12, 13 | 0.644 | 0.504 | 0.785 | 0.483 | −2.453 | 0.080 | 0.353 | 0.895 | 139.7 |
| | | 148b- | | | | | | | | | | | |
| | | 3p/hsa- | | | | | | | | | | | |
| | | miR-941 | | | | | | | | | | | |
| 60 | 1 | hsa-miR- | 5 | 2, 9 | 0.647 | 0.508 | 0.786 | 0.535 | −2.948 | 0.083 | 0.3 | 0.667 | 299.7 |
| | | 221- | | | | | | | | | | | |
| | | 5p/hsa- | | | | | | | | | | | |
| | | miR-760 | | | | | | | | | | | |
| 60 | 2 | hsa-miR- | 4 | 3, 6 | 0.647 | 0.508 | 0.786 | 0.535 | −2.948 | 0.083 | 0.198 | 0.5 | 105.1 |
| | | 330- | | | | | | | | | | | |
| | | 5p/hsa- | | | | | | | | | | | |
| | | miR- | | | | | | | | | | | |
| | | 550a-3- | | | | | | | | | | | |
| | | 5p-hsa- | | | | | | | | | | | |
| | | miR- | | | | | | | | | | | |
| | | 550a-5p | | | | | | | | | | | |
| 60 | 3 | hsa-miR- | 2 | 4, 5 | 0.647 | 0.508 | 0.786 | 0.535 | −2.948 | 0.083 | 0.154 | 0.625 | 273.8 |
| | | 501- | | | | | | | | | | | |
| | | 3p/hsa- | | | | | | | | | | | |
| | | miR-485- | | | | | | | | | | | |
| | | 5p | | | | | | | | | | | |
| 60 | 6 | hsa-miR- | 2 | 7, 8 | 0.647 | 0.508 | 0.786 | 0.535 | −2.948 | 0.083 | 0.182 | 0.606 | 59.0 |
| | | 99b- | | | | | | | | | | | |
| | | 3p/hsa- | | | | | | | | | | | |
| | | miR- | | | | | | | | | | | |
| | | 518e-5p- | | | | | | | | | | | |
| | | hsa-miR- | | | | | | | | | | | |
| | | 519a-5p- | | | | | | | | | | | |
| | | hsa-miR- | | | | | | | | | | | |
| | | 519b-5p- | | | | | | | | | | | |
| | | hsa-miR- | | | | | | | | | | | |
| | | 519c-5p- | | | | | | | | | | | |
| | | hsa-miR- | | | | | | | | | | | |
| | | 522-5p- | | | | | | | | | | | |
| | | hsa-miR- | | | | | | | | | | | |
| | | 523-5p | | | | | | | | | | | |
| 61 | 1 | hsa-miR- | 4 | 2, 7 | 0.629 | 0.502 | 0.755 | 0.520 | −3.035 | 0.061 | 0.307 | 0.818 | 114.6 |
| | | 330- | | | | | | | | | | | |
| | | 5p/hsa- | | | | | | | | | | | |
| | | miR-223- | | | | | | | | | | | |
| | | 3p | | | | | | | | | | | |
| 61 | 2 | hsa-miR- | 3 | 3, 6 | 0.629 | 0.502 | 0.755 | 0.520 | −3.035 | 0.061 | 0.258 | 0.647 | 288.1 |
| | | 7-5p/hsa- | | | | | | | | | | | |
| | | miR-485- | | | | | | | | | | | |
| | | 5p | | | | | | | | | | | |
| 61 | 3 | hsa-miR- | 2 | 4, 5 | 0.629 | 0.502 | 0.755 | 0.520 | −3.035 | 0.061 | 0.22 | 0.636 | 402.5 |
| | | 511- | | | | | | | | | | | |
| | | 5p/hsa- | | | | | | | | | | | |
| | | miR- | | | | | | | | | | | |
| | | 148a-5p | | | | | | | | | | | |
| 62 | 1 | hsa-miR- | 5 | 2, 9 | 0.64 | 0.506 | 0.774 | 0.535 | −3.037 | 0.070 | 0.296 | 0.846 | 359.8 |
| | | 7-5p/hsa- | | | | | | | | | | | |
| | | miR-485- | | | | | | | | | | | |
| | | 5p | | | | | | | | | | | |
| 62 | 2 | hsa-miR- | 4 | 3, 6 | 0.64 | 0.506 | 0.774 | 0.535 | −3.037 | 0.070 | 0.143 | 0.405 | 51.6 |
| | | 1306- | | | | | | | | | | | |
| | | 3p/hsa- | | | | | | | | | | | |
| | | miR- | | | | | | | | | | | |
| | | 664a-5p | | | | | | | | | | | |
| 62 | 3 | hsa-miR- | 2 | 4, 5 | 0.64 | 0.506 | 0.774 | 0.535 | −3.037 | 0.070 | 0.0244 | 0.467 | 152.3 |
| | | 30a- | | | | | | | | | | | |
| | | 5p/hsa- | | | | | | | | | | | |
| | | miR-941 | | | | | | | | | | | |
| 62 | 6 | hsa-miR- | 2 | 7, 8 | 0.64 | 0.506 | 0.774 | 0.535 | −3.037 | 0.070 | 0.519 | 0.16 | 164.7 |
| | | 30c- | | | | | | | | | | | |
| | | 5p/hsa- | | | | | | | | | | | |
| | | miR-27b- | | | | | | | | | | | |
| | | 3p | | | | | | | | | | | |

The data found in Table 7 will be used to model binary trees to predict placental dysfunction. In the tree model data of Table 7, the model column represents the ordered ranking of the current tree model. Each model represents an analysis using the panel of biomarkers listed in Table 6. Each row within a model represents a reversal analysis from a particular node. The node associated with a particular analysis is identified in the second column labeled “node”. The node column represents the node numbering within each tree. The reversal column shows the miRNA ratio employed for classification at each node. The termini column represents the number of terminal nodes under a particular parent node. The children.L.R column indicates which nodes are the child nodes of the parent node. The AUC, lowerCI, and UpperCI columns show model performance represented by the area under the ROC curve along with its associated lower and upper 95% confidence intervals. The inhomogeneity column is the average contamination of all the terminal nodes in the final model, i.e. it is the sum of the proportion of cases in majority-control terminal nodes and the proportion of controls present in majority-case terminal nodes. Thus inhomogeneity is approximately double the average contamination of terminal nodes with non-majority subjects. The Zsum column represents the sum of the Z-score for the lowerCI of the AUC and the Z-score for the inhomogeneity column.

In further detail, the standard Z-score is calculated within each of the lowerCI and inhomogeneity columns by subtracting the column mean from each value and dividing the difference by the column standard deviation, assuming a normal distribution across the column values. For lowerCI, the greater the distance above the central tendency for a particular Z-score, the more highly it is ranked. For inhomogeneity, the further below the central tendency a Z-score may be, the more highly it is ranked. The final Zsum is a weighted sum of the two Z-scores.

The rank_sum column represents the sum of the inverse ranks of the AUC columns and the inhomogeneity column. The predict_L and predict_R columns represent the case proportions within left and right child nodes, respectively. Finally, the cut off column represents the value of the reversal at which subjects split and assigned from a parent node to its child nodes. This value changes depending on the type of assay that is performed.

### Example 5: Biomarker Panels Provide a Greater Predictive Power than Single Biomarker Pairs

The panels of nucleic acid biomarker pairs listed in Table 6 are more predictive (i.e., have a higher A.U.C value) of placental dysfunction than single biomarker pairs.

The predictive power of exemplary biomarker pairs was determined. The results are presented in Table 8.

TABLE 8
Predictive power of exemplary biomarker pairs.

| Reversal pair | lowerCI | AUC | upperCI |
|---|---|---|---|
| hsa.miR.98.5p_hsa.miR.485.5p | 0.562 | 0.640 | 0.718 |
| hsa.miR.7.5p_hsa.miR.485.5p | 0.597 | 0.674 | 0.750 |
| hsa.miR.30c.5p_hsa.miR.27b.3p | 0.527 | 0.606 | 0.685 |
| hsa.miR.1306.3p_hsa.miR.664a.5p | 0.555 | 0.632 | 0.709 |
| hsa.miR.221.5p_hsa.miR.760 | 0.546 | 0.627 | 0.709 |
| hsa.miR.330.5p_hsa.miR.223.3p | 0.507 | 0.590 | 0.674 |

A comparison of the predictive power (AUC value) of the individual biomarkers with the predictive power of the panels of biomarkers listed in Table 6 shows that the panels outperform individual biomarkers pairs for predicting placental dysfunction. That is, the panels of biomarkers listed in Table 6 possess a higher AUC value than the reversal pairs listed in Table 8.

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A panel of isolated nucleic acid biomarkers comprising a combination of nucleic acid biomarker pairs as listed in Table 6.

2. The panel of claim 1, wherein the combination of nucleic acid biomarker pairs is selected from the group consisting of: biomarkers comprising hsa-miR-98-5p/hsa-miR-485-5p; hsa-miR-1306-3p/hsa-miR-1224-5p; hsa-miR-330-5p/hsa-miR-550a-3-5p-hsa-miR-550a-5p; and hsa-miR-221-5p/hsa-miR-197-3p; biomarkers comprising hsa-miR-7-5p/hsa-miR-485-5p; and hsa-miR-1306-3p/hsa-miR-664a-5p; hsa-miR-99b-3p/hsa-miR-760; hsa-miR-98-5p/hsa-miR-483-3p; biomarkers comprising hsa-miR-30c-5p/hsa-miR-27b-3p; hsa-miR-433-3p/hsa-miR-345-5p; hsa-miR-15b-5p/hsa-miR-197-3p; and hsa-miR-363-3p/hsa-miR-485-5p; biomarkers comprising hsa-miR-1306-3p/hsa-miR-664a-5p; hsa-miR-142-5p/hsa-miR-485-5p; hsa-miR-15b-5p/hsa-miR-197-3p; hsa-miR-7-5p/hsa-miR-485-5p; and hsa-miR-433-3p/hsa-miR-345-5p; biomarkers comprising hsa-miR-1306-3p/hsa-miR-664a-5p; hsa-miR-378g/hsa-miR-485-5p; hsa-miR-30c-5p/hsa-miR-100-5p; hsa-miR-30c-5p/hsa-miR-30a-5p; and hsa-miR-7-5p/hsa-let-7c-5p; biomarkers comprising hsa-miR-7-5p/hsa-miR-485-5p; hsa-miR-1306-3p/hsa-miR-664a-5p; hsa-miR-433-3p/hsa-miR-345-5p; and hsa-miR-30c-5p/hsa-miR-27b-3p; biomarkers comprising hsa-miR-7-5p/hsa-miR-485-5p; hsa-miR-1306-3p/hsa-miR-664a-5p; hsa-miR-7-5p/hsa-miR-4446-3p; and hsa-miR-221-5p/hsa-miR-760; biomarkers comprising hsa-miR-7-5p/hsa-miR-485-5p; hsa-miR-1306-3p/hsa-miR-664a-5p; and hsa-miR-501-3p/hsa-miR-484; biomarkers comprising hsa-miR-7-5p/hsa-miR-485-5p; hsa-miR-1306-3p/hsa-miR-664a-5p; hsa-miR-99b-3p/hsa-miR-760; and hsa-miR-221-5p/hsa-miR-760; hsa-miR-7-5p/hsa-miR-485-5p; hsa-miR-1306-3p/hsa-miR-664a-5p; hsa-miR-99b-3p/hsa-miR-379-5p; and hsa-miR-221-5p/hsa-miR-197-3p; biomarkers comprising hsa-miR-7-5p/hsa-miR-485-5p; hsa-miR-1-3p/hsa-miR-345-5p; hsa-miR-99b-3p/hsa-miR-150-3p; and hsa-miR-30c-5p/hsa-miR-146a-5p; biomarkers comprising hsa-miR-7-5p/hsa-miR-485-5p; hsa-miR-1306-3p/hsa-miR-664a-5p; and hsa-miR-628-3p/hsa-miR-19b-3p; hsa-miR-7-5p/hsa-miR-485-5p; hsa-miR-1306-3p/hsa-miR-664a-5p; hsa-miR-30c-5p/hsa-miR-7-5p; and hsa-miR-221-5p/hsa-miR-760; biomarkers comprising hsa-miR-7-5p/hsa-miR-485-5p; hsa-miR-7-5p/hsa-miR-4446-3p; hsa-miR-330-5p/hsa-miR-550a-3-5p-hsa-miR-550a-5p; hsa-let-7i-5p/hsa-miR-191-5p; hsa-miR-760/hsa-miR-345-5p; and hsa-miR-125a-3p/hsa-miR-379-5p; biomarkers comprising hsa-miR-98-5p/hsa-miR-485-5p; hsa-miR-1306-3p/hsa-miR-664a-5p; hsa-miR-99b-3p/hsa-miR-22-3p; and hsa-miR-221-5p/hsa-miR-760; biomarkers comprising hsa-miR-7-5p/hsa-miR-485-5p; hsa-miR-433-3p/hsa-miR-345-5p; hsa-miR-15b-5p/hsa-miR-197-3p; and hsa-miR-330-5p/hsa-miR-223-3p; biomarkers comprising hsa-miR-1306-3p/hsa-miR-664a-5p; hsa-miR-330-5p/hsa-miR-550a-3-5p-hsa-miR-550a-5p; hsa-miR-378g/hsa-miR-485-5p; hsa-miR-501-3p/hsa-miR-4446-3p; hsa-miR-30c-5p/hsa-miR-7-5p; and hsa-miR-20a-5p/hsa-miR-486-3p; biomarkers comprising hsa-miR-7-5p/hsa-miR-485-5p; hsa-miR-323b-3p/hsa-mir-320c-2; hsa-miR-99b-3p/hsa-miR-340-5p; hsa-miR-221-5p/hsa-miR-760; biomarkers comprising hsa-miR-7-5p/hsa-miR-485-5p; hsa-miR-1306-3p/hsa-miR-664a-5p; hsa-miR-433-3p/hsa-miR-345-5p; and hsa-miR-221-5p/hsa-miR-760; biomarkers comprising hsa-miR-7-5p/hsa-miR-485-5p; hsa-miR-330-5p/hsa-miR-550a-3-5p-hsa-miR-550a-5p; and hsa-miR-1306-3p/hsa-miR-1224-5p; biomarkers comprising hsa-miR-7-5p/hsa-miR-485-5p; hsa-miR-1306-3p/hsa-miR-664a-5p; hsa-miR-99b-3p/hsa-miR-760; and hsa-miR-7-5p/hsa-miR-4446-3p; biomarkers comprising hsa-miR-7-5p/hsa-miR-485-5p; hsa-miR-221-5p/hsa-miR-760; hsa-miR-1260b/hsa-mir-320c-2; and hsa-miR-1306-3p/hsa-miR-664a-5p; biomarkers comprising hsa-miR-1306-3p/hsa-miR-664a-5p; hsa-miR-378g/hsa-miR-485-5p; hsa-miR-30c-5p/hsa-miR-100-5p; hsa-miR-501-3p/hsa-miR-484; and hsa-miR-221-5p/hsa-miR-760; biomarkers comprising hsa-miR-7-5p/hsa-miR-485-5p; hsa-miR-221-5p/hsa-miR-760; hsa-miR-1306-3p/hsa-miR-148a-5p; hsa-miR-30c-5p/hsa-miR-320b; and hsa-miR-378e/hsa-miR-140-5p; biomarkers comprising hsa-miR-30c-5p/hsa-miR-27b-3p; hsa-miR-199a-3p-hsa-miR-199b-3p/hsa-miR-485-5p; and hsa-miR-1306-3p/hsa-miR-664a-5p; biomarkers comprising hsa-miR-7-5p/hsa-miR-485-5p; hsa-miR-30c-5p/hsa-miR-125a-5p; and hsa-miR-1-3p/hsa-miR-345-5p; biomarkers comprising hsa-miR-7-5p/hsa-miR-485-5p; hsa-miR-1-3p/hsa-miR-345-5p; hsa-miR-99b-3p/hsa-miR-483-3p; and hsa-miR-30c-5p/hsa-miR-10b-5p; biomarkers comprising hsa-miR-1306-3p/hsa-miR-664a-5p; hsa-miR-7-5p/hsa-miR-485-5p; hsa-miR-433-3p/hsa-miR-345-5p; hsa-miR-30c-5p/hsa-miR-27b-3p; and hsa-miR-99b-3p/hsa-miR-148a-5p; biomarkers comprising hsa-miR-7-5p/hsa-miR-485-5p; hsa-miR-1306-3p/hsa-miR-664a-5p; hsa-miR-1-3p/hsa-miR-345-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p; biomarkers comprising hsa-miR-7-5p/hsa-miR-485-5p; hsa-miR-1306-3p/hsa-miR-664a-5p; hsa-miR-99b-3p/hsa-miR-760; and hsa-miR-760/hsa-miR-345-5p; biomarkers comprising hsa-miR-7-5p/hsa-miR-485-5p; hsa-miR-378e/hsa-miR-483-3p; hsa-miR-194-5p/hsa-miR-486-3p; hsa-miR-26b-5p/hsa-miR-769-5p; hsa-miR-99b-3p/hsa-miR-760; hsa-miR-99b-3p/hsa-miR-144-5p; and hsa-miR-221-5p/hsa-miR-197-3p; biomarkers comprising hsa-miR-378g/hsa-miR-485-5p; hsa-miR-628-3p/hsa-miR-19b-3p; and hsa-miR-221-5p/hsa-miR-760; biomarkers comprising hsa-miR-1260b/hsa-mir-320c-2; and hsa-miR-433-3p/hsa-miR-345-5p; biomarkers comprising hsa-miR-330-5p/hsa-miR-550a-3-5p-hsa-miR-550a-5p; hsa-miR-1-3p/hsa-miR-345-5p; and hsa-miR-760/hsa-miR-345-5p; biomarkers comprising hsa-miR-7-5p/hsa-miR-485-5p; hsa-miR-760/hsa-miR-345-5p; hsa-miR-221-5p/hsa-miR-1908-5p; and hsa-miR-30c-5p/hsa-miR-7-5p; biomarkers comprising hsa-miR-1306-3p/hsa-miR-1224-5p; hsa-miR-99b-3p/hsa-miR-760; and hsa-miR-543/hsa-miR-345-5p; biomarkers comprising hsa-miR-330-5p/hsa-miR-550a-3-5p-hsa-miR-550a-5p; and hsa-miR-30c-5p/hsa-miR-7-5p; biomarkers comprising hsa-miR-330-5p/hsa-miR-550a-3-5p-hsa-miR-550a-5p; and hsa-miR-1-3p/hsa-miR-345-5p; biomarkers comprising hsa-miR-330-5p/hsa-miR-550a-3-5p-hsa-miR-550a-5p; hsa-miR-30c-5p/hsa-miR-10b-5p; and hsa-miR-433-3p/hsa-miR-345-5p; biomarkers comprising hsa-miR-1306-3p/hsa-miR-664a-5p; hsa-miR-1-3p/hsa-miR-345-5p; and hsa-miR-628-3p/hsa-miR-19b-3p; biomarkers comprising hsa-miR-1306-3p/hsa-miR-664a-5p; hsa-miR-99b-3p/hsa-miR-760; and hsa-miR-323b-3p/hsa-mir-320c-2; biomarkers comprising hsa-miR-221-5p/hsa-miR-760; hsa-miR-330-5p/hsa-miR-550a-3-5p-hsa-miR-550a-5p; hsa-miR-1306-3p/hsa-miR-664a-5p; and hsa-miR-766-3p/hsa-miR-148a-5p; biomarkers comprising hsa-miR-1306-3p/hsa-miR-664a-5p; hsa-miR-99b-3p/hsa-miR-760; and hsa-miR-7-5p/hsa-let-7c-5p; biomarkers comprising hsa-miR-7-5p/hsa-miR-485-5p; hsa-miR-1260b/hsa-mir-320c-2; and hsa-miR-433-3p/hsa-miR-345-5p; biomarkers comprising hsa-miR-511-5p/hsa-miR-148a-5p; hsa-miR-23b-5p/hsa-miR-2110; hsa-miR-221-5p/hsa-miR-760; hsa-miR-323b-3p/hsa-mir-320c-2; and hsa-miR-20a-5p/hsa-miR-486-3p; biomarkers comprising hsa-miR-221-5p/hsa-miR-760; hsa-miR-15b-5p/hsa-miR-197-3p; hsa-miR-194-5p/hsa-miR-486-3p; and hsa-miR-1306-3p/hsa-miR-335-3p; biomarkers comprising hsa-miR-1306-3p/hsa-miR-664a-5p; hsa-miR-99b-3p/hsa-miR-485-5p; and hsa-miR-221-5p/hsa-miR-197-3p; biomarkers comprising hsa-miR-221-5p/hsa-miR-197-3p; hsa-miR-1306-3p/hsa-miR-150-3p; and hsa-miR-15b-5p/hsa-miR-197-3p; biomarkers comprising hsa-miR-330-5p/hsa-miR-550a-3-5p-hsa-miR-550a-5p; and hsa-miR-30c-5p/hsa-miR-21-5p; biomarkers comprising hsa-miR-99b-3p/hsa-miR-483-3p; hsa-miR-330-5p/hsa-miR-550a-3-5p-hsa-miR-550a-5p; hsa-miR-30c-5p/hsa-miR-100-5p; hsa-miR-30c-5p/hsa-miR-425-5p; and hsa-miR-511-5p/hsa-miR-148a-5p; biomarkers comprising hsa-miR-7-5p/hsa-miR-4446-3p; hsa-miR-1260b/hsa-mir-320c-2; hsa-miR-1260b/hsa-mir-320c-2; and hsa-miR-1260b/hsa-mir-320c-2; biomarkers comprising hsa-miR-330-5p/hsa-miR-550a-3-5p-hsa-miR-550a-5p; and hsa-miR-30c-5p/hsa-let-7g-5p; biomarkers comprising hsa-miR-1306-3p/hsa-miR-1224-5p; hsa-miR-142-5p/hsa-miR-485-5p; and hsa-miR-221-5p/hsa-miR-197-3p; biomarkers comprising hsa-miR-330-5p/hsa-miR-550a-3-5p-hsa-miR-550a-5p; and hsa-miR-30c-5p/hsa-miR-625-3p; biomarkers comprising hsa-miR-23b-5p/hsa-miR-2110; hsa-miR-30c-5p/hsa-miR-125a-5p; hsa-miR-760/hsa-miR-345-5p; and hsa-miR-99b-3p/hsa-miR-150-3p; biomarkers comprising hsa-miR-378g/hsa-miR-485-5p; hsa-miR-30c-5p/hsa-miR-27b-3p; hsa-miR-766-3p/hsa-miR-148a-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p; biomarkers comprising hsa-miR-221-5p/hsa-miR-760; hsa-miR-99b-3p/hsa-miR-340-5p; and hsa-miR-1306-3p/hsa-miR-150-3p; biomarkers comprising hsa-miR-330-5p/hsa-miR-550a-3-5p-hsa-miR-550a-5p; and hsa-miR-323b-3p/hsa-mir-320c-2; biomarkers comprising hsa-miR-148b-3p/hsa-miR-941; hsa-miR-378g/hsa-miR-485-5p; hsa-miR-99b-3p/hsa-miR-760; hsa-miR-221-5p/hsa-miR-760; and hsa-miR-30c-5p/hsa-miR-27b-3p; biomarkers comprising hsa-miR-330-5p/hsa-miR-550a-3-5p-hsa-miR-550a-5p; hsa-miR-501-3p/hsa-miR-485-5p; and hsa-miR-99b-3p/hsa-miR-518e-5p-hsa-miR-519a-5p-hsa-miR-519b-5p-hsa-miR-519c-5p-hsa-miR-522-5p-hsa-miR-523-5p; biomarkers comprising hsa-miR-7-5p/hsa-miR-485-5p; and hsa-miR-511-5p/hsa-miR-148a-5p; biomarkers comprising hsa-miR-1306-3p/hsa-miR-664a-5p; hsa-miR-30a-5p/hsa-miR-941; and hsa-miR-30c-5p/hsa-miR-27b-3p.

3-63. (canceled)

64. The panel of claim 2, wherein the combination of nucleic acid biomarker pairs consists of one or more of the combinations.

65-67. (canceled)

68. A composition of labeled and/or amplified nucleic acid molecules, wherein said labeled and/or amplified nucleic acid molecules correspond to the panel of isolated nucleic acid biomarkers of claim 1.

69. A method for determining a pregnant female's risk of developing placental dysfunction during pregnancy comprising measuring amounts of the combination of nucleic acid biomarker pairs from the panel of isolated nucleic acid biomarkers of claim 1 in a biological sample obtained from the pregnant female, calculating a risk score based upon the measured amounts to determine the pregnant female's risk of developing placental dysfunction, and providing a risk score corresponding to the pregnant female's risk of developing placental dysfunction.

70. A method for determining a pregnant female's risk of developing placental dysfunction during pregnancy comprising producing labeled and/or amplified nucleic acid molecules that correspond to the composition of labeled and/or amplified nucleic acid molecules of claim 69 in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

71. The method of claim 69, wherein the biological sample is obtained between 17 and 29 weeks of gestational age at blood draw.

72-75. (canceled)

76. The method of claim 69, wherein the risk score is calculated based on a ratio of data values.

77. The method of claim 76, wherein data transformation is applied before or after the ratio is determined.

78. The method of claim 69, wherein the method determines the pregnant female's risk of developing placental dysfunction early or later in the pregnancy.

79-81. (canceled)

82. A method of treating or preventing placental dysfunction in a pregnant female, the method comprising:
(a) detecting the panel of isolated nucleic acid biomarkers of claim 1 in a biological sample obtained from a pregnant female;
(b) calculating a risk score based upon measured levels of the isolated nucleic acid biomarkers; and
(c) administering one or more therapies to the pregnant female based on the risk score, to treat or prevent placental dysfunction.

83. A method of treating or preventing placental dysfunction in a pregnant female, the method comprising:
(a) detecting the composition of labeled and/or amplified nucleic acid molecules of claim 68 from a biological sample obtained from a pregnant female;
(b) calculating a risk score based upon measured levels of the labeled and/or amplified nucleic acid molecules; and
(c) administering one or more therapies to the pregnant female based on the risk score, to treat or prevent placental dysfunction.

84. The method of claim 82, wherein the nucleic acid biomarkers are normalized.

85. The method of claim 84, wherein normalization comprises a reversal of the isolated nucleic acid biomarkers.

86. The method of claim 85, wherein the reversal comprises an upregulated biomarker and a downregulated biomarker.

87. The method of any one of claim 85, wherein the risk score is calculated based on the reversal of data values.

88-97. (canceled)

98. The method of claim 69, wherein calculating the risk score is performed using a binary decision tree analysis.

99-104. (canceled)

105. A method for treating or preventing placental dysfunction in a pregnant female, the method comprising:
(a) detecting the panel of isolated nucleic acid biomarkers of claim 1 in a biological sample obtained from a pregnant female;
(b) providing a risk score based upon measured levels of the labeled and/or amplified nucleic acid molecules;
(c) determining that said pregnant female has an increased likelihood of placental dysfunction based at least in part on said risk score exceeding a reference score; and
(d) recommending, facilitating, prescribing, or administering one or more therapies to the pregnant female based on the risk score, to treat or prevent placental dysfunction.

106-126. (canceled)
